SEQUENCE LISTING (479 sequences)

SEQ ID NO: 1 - protein, 21 aa, synthetic construct
  REGION 1..21: note = Synthetic
PPGPPGPPGPPGPPGPPGPPG

SEQ ID NO: 2 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPPGPPG

SEQ ID NO: 3 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGPPG

SEQ ID NO: 4 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PPGPPGPPGPPGPPGPPGPPG

SEQ ID NO: 5 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGPPG

SEQ ID NO: 6 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGPPG

SEQ ID NO: 7 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
PPGPPGPPGPPGPPGPPGPPG

SEQ ID NO: 8 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGPPG

SEQ ID NO: 9 - protein, 22 aa, synthetic construct
  REGION 1..22: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 21: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGPPPG

SEQ ID NO: 10 - protein, 21 aa, synthetic construct
  REGION 1..21: note = Synthetic
PPGPPGCPGPPGPPGPPGPPG

SEQ ID NO: 11 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGCPGPPGPPGPPGPPG

SEQ ID NO: 12 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPCGPPGPPGPPGPPG

SEQ ID NO: 13 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPCGPPGPPGPPGPPG

SEQ ID NO: 14 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPCGPPGPPGPPGPPG

SEQ ID NO: 15 - protein, 21 aa, synthetic construct
  REGION 1..21: note = Synthetic
CPGPPGPPGPPGPPGPPGPPG

SEQ ID NO: 16 - protein, 21 aa, synthetic construct
  REGION 1..21: note = Synthetic
PCGPPGPPGPPGPPGPPGPPG

SEQ ID NO: 17 - protein, 21 aa, synthetic construct
  REGION 1..21: note = Synthetic
PPGCPGPPGPPGPPGPPGPPG

SEQ ID NO: 18 - protein, 21 aa, synthetic construct
  REGION 1..21: note = Synthetic
PPGPCGPPGPPGPPGPPGPPG

SEQ ID NO: 19 - protein, 21 aa, synthetic construct
  REGION 1..21: note = Synthetic
PPGPPGPCGPPGPPGPPGPPG

SEQ ID NO: 20 - protein, 21 aa, synthetic construct
  REGION 1..21: note = Synthetic
PPGPPGPPGCPGPPGPPGPPG

SEQ ID NO: 21 - protein, 21 aa, synthetic construct
  REGION 1..21: note = Synthetic
PPGPPGPPGPCGPPGPPGPPG

SEQ ID NO: 22 - protein, 21 aa, synthetic construct
  REGION 1..21: note = Synthetic
PPGPPGPPGPPGCPGPPGPPG

SEQ ID NO: 23 - protein, 21 aa, synthetic construct
  REGION 1..21: note = Synthetic
PPGPPGPPGPPGPCGPPGPPG

SEQ ID NO: 24 - protein, 21 aa, synthetic construct
  REGION 1..21: note = Synthetic
PPGPPGPPGPPGPPGCPGPPG

SEQ ID NO: 25 - protein, 21 aa, synthetic construct
  REGION 1..21: note = Synthetic
PPGPPGPPGPPGPPGPCGCPG

SEQ ID NO: 26 - protein, 21 aa, synthetic construct
  REGION 1..21: note = Synthetic
PPGPPGPPGPPGPPGPPGCPG

SEQ ID NO: 27 - protein, 21 aa, synthetic construct
  REGION 1..21: note = Synthetic
PPGPPGPPGPPGPPGPPGPCG

SEQ ID NO: 28 - protein, 21 aa, synthetic construct
  REGION 1..21: note = Synthetic
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
CPGPPGPPGPPGPPGPPGPPG

SEQ ID NO: 29 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PCGPPGPPGPPGPPGPPGPPG

SEQ ID NO: 30 - protein, 21 aa, synthetic construct
  REGION 1..21: note = Synthetic
  MOD_RES 1: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGCPGPPGPPGPPGPPGPPG

SEQ ID NO: 31 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPCGPPGPPGPPGPPGPPG

SEQ ID NO: 32 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPCGPPGPPGPPGPPG

SEQ ID NO: 33 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGCPGPPGPPGPPG

SEQ ID NO: 34 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPCGPPGPPGPPG

SEQ ID NO: 35 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 16: note = Hydroxyproline (Hyp)
  MOD_RES 19: note = Hydroxyproline (Hyp)
PPGPPGPPGPPGCPGPPGPPG

SEQ ID NO: 36 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPCGPPGPPG

SEQ ID NO: 37 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 19: note = Hydroxyproline (Hyp)
PPGPPGPPGPPGPPGCPGPPG

SEQ ID NO: 38 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPCGPPG

SEQ ID NO: 39 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
PPGPPGPPGPPGPPGPPGCPG

SEQ ID NO: 40 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPPGPCG

SEQ ID NO: 41 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
CPGPPGPPGPPGPPGPPGPPG

SEQ ID NO: 42 - protein, 21 aa, synthetic construct
  REGION 1..21: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PCGPPGPPGPPGPPGPPGPPG

SEQ ID NO: 43 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGCPGPPGPPGPPGPPGPPG

SEQ ID NO: 44 - protein, 21 aa, synthetic construct
  REGION 1..21: note = Synthetic
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPCGPPGPPGPPGPPGPPG

SEQ ID NO: 45 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGCPGPPGPPGPPGPPG

SEQ ID NO: 46 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGCPGPPGPPGPPG

SEQ ID NO: 47 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPCGPPGPPGPPG

SEQ ID NO: 48 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGCPGPPGPPG

SEQ ID NO: 49 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPCGPPGPPG

SEQ ID NO: 50 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGCPGPPG

SEQ ID NO: 51 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPCGPPG

SEQ ID NO: 52 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGCPG

SEQ ID NO: 53 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPCG

SEQ ID NO: 54 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
CPGPPGPPGPPGPPGPPGPPG

SEQ ID NO: 55 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PCGPPGPPGPPGPPGPPGPPG

SEQ ID NO: 56 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGCPGPPGPPGPPGPPGPPG

SEQ ID NO: 57 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPCGPPGPPGPPGPPGPPG

SEQ ID NO: 58 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGCPGPPGPPGPPGPPG

SEQ ID NO: 59 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGCPGPPGPPGPPG

SEQ ID NO: 60 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPCGPPGPPGPPG

SEQ ID NO: 61 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGCPGPPGPPG

SEQ ID NO: 62 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPCGPPGPPG

SEQ ID NO: 63 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGCPGPPG

SEQ ID NO: 64 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPCGPPG

SEQ ID NO: 65 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGCPG

SEQ ID NO: 66 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPCG

SEQ ID NO: 67 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
CPGPPGPPGPPGPPGPPGPPG

SEQ ID NO: 68 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PCGPPGPPGPPGPPGPPGPPG

SEQ ID NO: 69 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGCPGPPGPPGPPGPPGPPG

SEQ ID NO: 70 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPCGPPGPPGPPGPPGPPG

SEQ ID NO: 71 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGCPGPPGPPGPPGPPG

SEQ ID NO: 72 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPCGPPGPPGPPGPPG

SEQ ID NO: 73 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGCPGPPGPPGPPG

SEQ ID NO: 74 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPCGPPGPPGPPG

SEQ ID NO: 75 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGCPGPPGPPG

SEQ ID NO: 76 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPCGPPGPPG

SEQ ID NO: 77 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGCPGPPG

SEQ ID NO: 78 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPCGPPG

SEQ ID NO: 79 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGCPG

SEQ ID NO: 80 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPCG

SEQ ID NO: 81 - protein, 21 aa, synthetic construct
  REGION 1..21: note = Synthetic
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
CPGPPGPPGPPGPPGPPGPPG

SEQ ID NO: 82 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PCGPPGPPGPPGPPGPPGPPG

SEQ ID NO: 83 - protein, 21 aa, synthetic construct
  REGION 1..21: note = Synthetic
  MOD_RES 1: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGCPGPPGPPGPPGPPGPPG

SEQ ID NO: 84 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPCGPPGPPGPPGPPGPPG

SEQ ID NO: 85 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGCPGPPGPPGPPGPPG

SEQ ID NO: 86 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPCGPPGPPGPPGPPG

SEQ ID NO: 87 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGCPGPPGPPGPPG

SEQ ID NO: 88 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPCGPPGPPGPPG

SEQ ID NO: 89 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGCPGPPGPPG

SEQ ID NO: 90 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPCGPPGPPG

SEQ ID NO: 91 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGCPGPPG

SEQ ID NO: 92 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPCGPPG

SEQ ID NO: 93 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGCPG

SEQ ID NO: 94 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPCG

SEQ ID NO: 95 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
CPGPPGPPGPPGPPGPPGPPG

SEQ ID NO: 96 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PCGPPGPPGPPGPPGPPGPPG

SEQ ID NO: 97 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGCPGPPGPPGPPGPPGPPG

SEQ ID NO: 98 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPCGPPGPPGPPGPPGPPG

SEQ ID NO: 99 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGCPGPPGPPGPPGPPG

SEQ ID NO: 100 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGCPGPPGPPGPPG

SEQ ID NO: 101 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPCGPPGPPGPPG

SEQ ID NO: 102 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGCPGPPGPPG

SEQ ID NO: 103 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPCGPPGPPG

SEQ ID NO: 104 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGCPGPPG

SEQ ID NO: 105 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPCGPPG

SEQ ID NO: 106 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGCPG

SEQ ID NO: 107 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPCG

SEQ ID NO: 108 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
CPGPPGPPGPPGPPGPPGPPG

SEQ ID NO: 109 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PCGPPGPPGPPGPPGPPGPPG

SEQ ID NO: 110 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGCPGPPGPPGPPGPPGPPG

SEQ ID NO: 111 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPCGPPGPPGPPGPPGPPG

SEQ ID NO: 112 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGCPGPPGPPGPPGPPG

SEQ ID NO: 113 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPCGPPGPPGPPGPPG

SEQ ID NO: 114 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGCPGPPGPPGPPG

SEQ ID NO: 115 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPCGPPGPPGPPG

SEQ ID NO: 116 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGCPGPPGPPG

SEQ ID NO: 117 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPCGPPGPPG

SEQ ID NO: 118 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGCPGPPG

SEQ ID NO: 119 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPCGPPG

SEQ ID NO: 120 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGCPG

SEQ ID NO: 121 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPCG

SEQ ID NO: 122 - protein, 21 aa, synthetic construct
  REGION 1..21: note = Synthetic
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
CPGPPGPPGPPGPPGPPGPPG

SEQ ID NO: 123 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PCGPPGPPGPPGPPGPPGPPG

SEQ ID NO: 124 - protein, 21 aa, synthetic construct
  REGION 1..21: note = Synthetic
  MOD_RES 1: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGCPGPPGPPGPPGPPGPPG

SEQ ID NO: 125 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPCGPPGPPGPPGPPGPPG

SEQ ID NO: 126 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGCPGPPGPPGPPGPPG

SEQ ID NO: 127 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPCGPPGPPGPPGPPG

SEQ ID NO: 128 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGCPGPPGPPGPPG

SEQ ID NO: 129 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPCGPPGPPGPPG

SEQ ID NO: 130 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGCPGPPGPPG

SEQ ID NO: 131 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPCGPPGPPG

SEQ ID NO: 132 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGCPGPPG

SEQ ID NO: 133 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPCGPPG

SEQ ID NO: 134 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGCPG

SEQ ID NO: 135 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPCG

SEQ ID NO: 136 - protein, 21 aa, synthetic construct
  REGION 1..21: note = Synthetic
PPGPPGMPGPPGPPGPPGPPG

SEQ ID NO: 137 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGMPGPPGPPGPPGPPG

SEQ ID NO: 138 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPMGPPGPPGPPGPPG

SEQ ID NO: 139 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPMGPPGPPGPPGPPG

SEQ ID NO: 140 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPMGPPGPPGPPGPPG

SEQ ID NO: 141 - protein, 21 aa, synthetic construct
  REGION 1..21: note = Synthetic
MPGPPGPPGPPGPPGPPGPPG

SEQ ID NO: 142 - protein, 21 aa, synthetic construct
  REGION 1..21: note = Synthetic
PMGPPGPPGPPGPPGPPGPPG

SEQ ID NO: 143 - protein, 21 aa, synthetic construct
  REGION 1..21: note = Synthetic
PPGMPGPPGPPGPPGPPGPPG

SEQ ID NO: 144 - protein, 21 aa, synthetic construct
  REGION 1..21: note = Synthetic
PPGPMGPPGPPGPPGPPGPPG

SEQ ID NO: 145 - protein, 21 aa, synthetic construct
  REGION 1..21: note = Synthetic
PPGPPGPMGPPGPPGPPGPPG

SEQ ID NO: 146 - protein, 21 aa, synthetic construct
  REGION 1..21: note = Synthetic
PPGPPGPPGMPGPPGPPGPPG

SEQ ID NO: 147 - protein, 21 aa, synthetic construct
  REGION 1..21: note = Synthetic
PPGPPGPPGPMGPPGPPGPPG

SEQ ID NO: 148 - protein, 21 aa, synthetic construct
  REGION 1..21: note = Synthetic
PPGPPGPPGPPGMPGPPGPPG

SEQ ID NO: 149 - protein, 21 aa, synthetic construct
  REGION 1..21: note = Synthetic
PPGPPGPPGPPGPMGPPGPPG

SEQ ID NO: 150 - protein, 21 aa, synthetic construct
  REGION 1..21: note = Synthetic
PPGPPGPPGPPGPPGMPGPPG

SEQ ID NO: 151 - protein, 21 aa, synthetic construct
  REGION 1..21: note = Synthetic
PPGPPGPPGPPGPPGPMGPPG

SEQ ID NO: 152 - protein, 21 aa, synthetic construct
  REGION 1..21: note = Synthetic
PPGPPGPPGPPGPPGPPGMPG

SEQ ID NO: 153 - protein, 21 aa, synthetic construct
  REGION 1..21: note = Synthetic
PPGPPGPPGPPGPPGPPGPMG

SEQ ID NO: 154 - protein, 21 aa, synthetic construct
  REGION 1..21: note = Synthetic
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
MPGPPGPPGPPGPPGPPGPPG

SEQ ID NO: 155 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PMGPPGPPGPPGPPGPPGPPG

SEQ ID NO: 156 - protein, 21 aa, synthetic construct
  REGION 1..21: note = Synthetic
  MOD_RES 1: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGMPGPPGPPGPPGPPGPPG

SEQ ID NO: 157 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPMGPPGPPGPPGPPGPPG

SEQ ID NO: 158 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPMGPPGPPGPPGPPG

SEQ ID NO: 159 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGMPGPPGPPGPPG

SEQ ID NO: 160 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPMGPPGPPGPPG

SEQ ID NO: 161 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 16: note = Hydroxyproline (Hyp)
  MOD_RES 19: note = Hydroxyproline (Hyp)
PPGPPGPPGPPGMPGPPGPPG

SEQ ID NO: 162 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPMGPPGPPG

SEQ ID NO: 163 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 19: note = Hydroxyproline (Hyp)
PPGPPGPPGPPGPPGMPGPPG

SEQ ID NO: 164 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPMGPPG

SEQ ID NO: 165 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
PPGPPGPPGPPGPPGPPGMPG

SEQ ID NO: 166 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPPGPMG

SEQ ID NO: 167 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
MPGPPGPPGPPGPPGPPGPPG

SEQ ID NO: 168 - protein, 21 aa, synthetic construct
  REGION 1..21: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PMGPPGPPGPPGPPGPPGPPG

SEQ ID NO: 169 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGMPGPPGPPGPPGPPGPPG

SEQ ID NO: 170 - protein, 21 aa, synthetic construct
  REGION 1..21: note = Synthetic
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPMGPPGPPGPPGPPGPPG

SEQ ID NO: 171 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGMPGPPGPPGPPGPPG

SEQ ID NO: 172 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGMPGPPGPPGPPG

SEQ ID NO: 173 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPMGPPGPPGPPG

SEQ ID NO: 174 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGMPGPPGPPG

SEQ ID NO: 175 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPMGPPGPPG

SEQ ID NO: 176 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGMPGPPG

SEQ ID NO: 177 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPMGPPG

SEQ ID NO: 178 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGMPG

SEQ ID NO: 179 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPMG

SEQ ID NO: 180 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
MPGPPGPPGPPGPPGPPGPPG

SEQ ID NO: 181 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PMGPPGPPGPPGPPGPPGPPG

SEQ ID NO: 182 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGMPGPPGPPGPPGPPGPPG

SEQ ID NO: 183 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPMGPPGPPGPPGPPGPPG

SEQ ID NO: 184 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGMPGPPGPPGPPGPPG

SEQ ID NO: 185 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGMPGPPGPPGPPG

SEQ ID NO: 186 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPMGPPGPPGPPG

SEQ ID NO: 187 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGMPGPPGPPG

SEQ ID NO: 188 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPMGPPGPPG

SEQ ID NO: 189 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGMPGPPG

SEQ ID NO: 190 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPMGPPG

SEQ ID NO: 191 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGMPG

SEQ ID NO: 192 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPMG

SEQ ID NO: 193 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
MPGPPGPPGPPGPPGPPGPPG

SEQ ID NO: 194 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PMGPPGPPGPPGPPGPPGPPG

SEQ ID NO: 195 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGMPGPPGPPGPPGPPGPPG

SEQ ID NO: 196 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPMGPPGPPGPPGPPGPPG

SEQ ID NO: 197 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGMPGPPGPPGPPGPPG

SEQ ID NO: 198 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPMGPPGPPGPPGPPG

SEQ ID NO: 199 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGMPGPPGPPGPPG

SEQ ID NO: 200 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPMGPPGPPGPPG

SEQ ID NO: 201 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGMPGPPGPPG

SEQ ID NO: 202 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPMGPPGPPG

SEQ ID NO: 203 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGMPGPPG

SEQ ID NO: 204 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPMGPPG

SEQ ID NO: 205 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGMPG

SEQ ID NO: 206 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPMG

SEQ ID NO: 207 - protein, 21 aa, synthetic construct
  REGION 1..21: note = Synthetic
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
MPGPPGPPGPPGPPGPPGPPG

SEQ ID NO: 208 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PMGPPGPPGPPGPPGPPGPPG

SEQ ID NO: 209 - protein, 21 aa, synthetic construct
  REGION 1..21: note = Synthetic
  MOD_RES 1: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGMPGPPGPPGPPGPPGPPG

SEQ ID NO: 210 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPMGPPGPPGPPGPPGPPG

SEQ ID NO: 211 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGMPGPPGPPGPPGPPG

SEQ ID NO: 212 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPMGPPGPPGPPGPPG

SEQ ID NO: 213 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGMPGPPGPPGPPG

SEQ ID NO: 214 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPMGPPGPPGPPG

SEQ ID NO: 215 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGMPGPPGPPG

SEQ ID NO: 216 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPMGPPGPPG

SEQ ID NO: 217 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGMPGPPG

SEQ ID NO: 218 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp) F
  MOD_RES 20: note = Hydroxyproline (Hyp)
PPGPPGPPGPPGPPGPMGPPG

SEQ ID NO: 219 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGMPG

SEQ ID NO: 220 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPMG

SEQ ID NO: 221 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
MPGPPGPPGPPGPPGPPGPPG

SEQ ID NO: 222 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PMGPPGPPGPPGPPGPPGPPG

SEQ ID NO: 223 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGMPGPPGPPGPPGPPGPPG

SEQ ID NO: 224 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPMGPPGPPGPPGPPGPPG

SEQ ID NO: 225 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGMPGPPGPPGPPGPPG

SEQ ID NO: 226 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGMPGPPGPPGPPG

SEQ ID NO: 227 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPMGPPGPPGPPG

SEQ ID NO: 228 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGMPGPPGPPG

SEQ ID NO: 229 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPMGPPGPPG

SEQ ID NO: 230 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGMPGPPG

SEQ ID NO: 231 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPMGPPG

SEQ ID NO: 232 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGMPG

SEQ ID NO: 233 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPMG

SEQ ID NO: 234 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
MPGPPGPPGPPGPPGPPGPPG

SEQ ID NO: 235 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PMGPPGPPGPPGPPGPPGPPG

SEQ ID NO: 236 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGMPGPPGPPGPPGPPGPPG

SEQ ID NO: 237 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPMGPPGPPGPPGPPGPPG

SEQ ID NO: 238 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGMPGPPGPPGPPGPPG

SEQ ID NO: 239 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPMGPPGPPGPPGPPG

SEQ ID NO: 240 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGMPGPPGPPGPPG

SEQ ID NO: 241 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPMGPPGPPGPPG

SEQ ID NO: 242 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGMPGPPGPPG

SEQ ID NO: 243 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPMGPPGPPG

SEQ ID NO: 244 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGMPGPPG

SEQ ID NO: 245 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPMGPPG

SEQ ID NO: 246 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGMPG

SEQ ID NO: 247 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPMG

SEQ ID NO: 248 - protein, 21 aa, synthetic construct
  REGION 1..21: note = Synthetic
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
MPGPPGPPGPPGPPGPPGPPG

SEQ ID NO: 249 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PMGPPGPPGPPGPPGPPGPPG

SEQ ID NO: 250 - protein, 21 aa, synthetic construct
  REGION 1..21: note = Synthetic
  MOD_RES 1: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGMPGPPGPPGPPGPPGPPG

SEQ ID NO: 251 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPMGPPGPPGPPGPPGPPG

SEQ ID NO: 252 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGMPGPPGPPGPPGPPG

SEQ ID NO: 253 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPMGPPGPPGPPGPPG

SEQ ID NO: 254 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGMPGPPGPPGPPG

SEQ ID NO: 255 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPMGPPGPPGPPG

SEQ ID NO: 256 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGMPGPPGPPG

SEQ ID NO: 257 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPMGPPGPPG

SEQ ID NO: 258 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGMPGPPG

SEQ ID NO: 259 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPMGPPG

SEQ ID NO: 260 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGMPG

SEQ ID NO: 261 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPMG

SEQ ID NO: 262 - protein, 21 aa, synthetic construct
  REGION 1..21: note = Synthetic
PPGPPGKPGPPGPPGPPGPPG

SEQ ID NO: 263 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGKPGPPGPPGPPGPPG

SEQ ID NO: 264 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPKGPPGPPGPPGPPG

SEQ ID NO: 265 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPKGPPGPPGPPGPPG

SEQ ID NO: 266 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPKGPPGPPGPPGPPG

SEQ ID NO: 267 - protein, 21 aa, synthetic construct
  REGION 1..21: note = Synthetic
KPGPPGPPGPPGPPGPPGPPG

SEQ ID NO: 268 - protein, 21 aa, synthetic construct
  REGION 1..21: note = Synthetic
PKGPPGPPGPPGPPGPPGPPG

SEQ ID NO: 269 - protein, 21 aa, synthetic construct
  REGION 1..21: note = Synthetic
PPGKPGPPGPPGPPGPPGPPG

SEQ ID NO: 270 - protein, 21 aa, synthetic construct
  REGION 1..21: note = Synthetic
PPGPKGPPGPPGPPGPPGPPG

SEQ ID NO: 271 - protein, 21 aa, synthetic construct
  REGION 1..21: note = Synthetic
PPGPPGPKGPPGPPGPPGPPG

SEQ ID NO: 272 - protein, 21 aa, synthetic construct
  REGION 1..21: note = Synthetic
PPGPPGPPGKPGPPGPPGPPG

SEQ ID NO: 273 - protein, 21 aa, synthetic construct
  REGION 1..21: note = Synthetic
PPGPPGPPGPKGPPGPPGPPG

SEQ ID NO: 274 - protein, 21 aa, synthetic construct
  REGION 1..21: note = Synthetic
PPGPPGPPGPPGKPGPPGPPG

SEQ ID NO: 275 - protein, 21 aa, synthetic construct
  REGION 1..21: note = Synthetic
PPGPPGPPGPPGPKGPPGPPG

SEQ ID NO: 276 - protein, 21 aa, synthetic construct
  REGION 1..21: note = Synthetic
PPGPPGPPGPPGPPGKPGPPG

SEQ ID NO: 277 - protein, 21 aa, synthetic construct
  REGION 1..21: note = Synthetic
PPGPPGPPGPPGPPGPKGPPG

SEQ ID NO: 278 - protein, 21 aa, synthetic construct
  REGION 1..21: note = Synthetic
PPGPPGPPGPPGPPGPPGKPG

SEQ ID NO: 279 - protein, 21 aa, synthetic construct
  REGION 1..21: note = Synthetic
PPGPPGPPGPPGPPGPPGPKG

SEQ ID NO: 280 - protein, 21 aa, synthetic construct
  REGION 1..21: note = Synthetic
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
KPGPPGPPGPPGPPGPPGPPG

SEQ ID NO: 281 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PKGPPGPPGPPGPPGPPGPPG

SEQ ID NO: 282 - protein, 21 aa, synthetic construct
  REGION 1..21: note = Synthetic
  MOD_RES 1: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGKPGPPGPPGPPGPPGPPG

SEQ ID NO: 283 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPKGPPGPPGPPGPPGPPG

SEQ ID NO: 284 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPKGPPGPPGPPGPPG

SEQ ID NO: 285 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGKPGPPGPPGPPG

SEQ ID NO: 286 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPKGPPGPPGPPG

SEQ ID NO: 287 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 16: note = Hydroxyproline (Hyp)
  MOD_RES 19: note = Hydroxyproline (Hyp)
PPGPPGPPGPPGKPGPPGPPG

SEQ ID NO: 288 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPKGPPGPPG

SEQ ID NO: 289 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 19: note = Hydroxyproline (Hyp)
PPGPPGPPGPPGPPGKPGPPG

SEQ ID NO: 290 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPKGPPG

SEQ ID NO: 291 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
PPGPPGPPGPPGPPGPPGKPG

SEQ ID NO: 292 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PPGPPGPPGPPGPPGPPGPKG

SEQ ID NO: 293 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
KPGPPGPPGPPGPPGPPGPPG

SEQ ID NO: 294 - protein, 21 aa, synthetic construct
  REGION 1..21: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PKGPPGPPGPPGPPGPPGPPG

SEQ ID NO: 295 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGKPGPPGPPGPPGPPGPPG

SEQ ID NO: 296 - protein, 21 aa, synthetic construct
  REGION 1..21: note = Synthetic
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPKGPPGPPGPPGPPGPPG

SEQ ID NO: 297 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGKPGPPGPPGPPGPPG

SEQ ID NO: 298 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGKPGPPGPPGPPG

SEQ ID NO: 299 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPKGPPGPPGPPG

SEQ ID NO: 300 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGKPGPPGPPG

SEQ ID NO: 301 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPKGPPGPPG

SEQ ID NO: 302 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGKPGPPG

SEQ ID NO: 303 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPKGPPG

SEQ ID NO: 304 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGKPG

SEQ ID NO: 305 - protein, 21 aa, synthetic construct
  REGION 1..21: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPKG

SEQ ID NO: 306 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
KPGPPGPPGPPGPPGPPGPPG

SEQ ID NO: 307 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PKGPPGPPGPPGPPGPPGPPG

SEQ ID NO: 308 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGKPGPPGPPGPPGPPGPPG

SEQ ID NO: 309 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPKGPPGPPGPPGPPGPPG

SEQ ID NO: 310 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGKPGPPGPPGPPGPPG

SEQ ID NO: 311 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGKPGPPGPPGPPG

SEQ ID NO: 312 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPKGPPGPPGPPG

SEQ ID NO: 313 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGKPGPPGPPG

SEQ ID NO: 314 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPKGPPGPPG

SEQ ID NO: 315 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGKPGPPG

SEQ ID NO: 316 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPKGPPG

SEQ ID NO: 317 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGKPG

SEQ ID NO: 318 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPKG

SEQ ID NO: 319 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
KPGPPGPPGPPGPPGPPGPPG

SEQ ID NO: 320 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PKGPPGPPGPPGPPGPPGPPG

SEQ ID NO: 321 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGKPGPPGPPGPPGPPGPPG

SEQ ID NO: 322 - protein, 21 aa, synthetic construct
  REGION 1..21: note = Synthetic
  MOD_RES 2: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPKGPPGPPGPPGPPGPPG

SEQ ID NO: 323 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGKPGPPGPPGPPGPPG

SEQ ID NO: 324 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPKGPPGPPGPPGPPG

SEQ ID NO: 325 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGKPGPPGPPGPPG

SEQ ID NO: 326 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPKGPPGPPGPPG

SEQ ID NO: 327 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGKPGPPGPPG

SEQ ID NO: 328 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 17: note = Fluoroproline (Flp)
  MOD_RES 20: note = Fluoroproline (Flp)
PPGPPGPPGPPGPKGPPGPPG

SEQ ID NO: 329 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGKPGPPG

SEQ ID NO: 330 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 20: note = Fluoroproline (Flp)
PPGPPGPPGPPGPPGPKGPPG

SEQ ID NO: 331 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGKPG

SEQ ID NO: 332 - protein, 21 aa, synthetic construct
  REGION 1..21: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGPKG

SEQ ID NO: 333 - protein, 21 aa, synthetic construct
  REGION 1..21: note = Synthetic
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
KPGPPGPPGPPGPPGPPGPPG

SEQ ID NO: 334 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PKGPPGPPGPPGPPGPPGPPG

SEQ ID NO: 335 - protein, 21 aa, synthetic construct
  REGION 1..21: note = Synthetic
  MOD_RES 1: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGKPGPPGPPGPPGPPGPPG

SEQ ID NO: 336 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPKGPPGPPGPPGPPGPPG

SEQ ID NO: 337 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGKPGPPGPPGPPGPPG

SEQ ID NO: 338 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPKGPPGPPGPPGPPG

SEQ ID NO: 339 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGKPGPPGPPGPPG

SEQ ID NO: 340 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPKGPPGPPGPPG

SEQ ID NO: 341 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGKPGPPGPPG

SEQ ID NO: 342 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPKGPPGPPG

SEQ ID NO: 343 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGKPGPPG

SEQ ID NO: 344 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPKGPPG

SEQ ID NO: 345 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGKPG

SEQ ID NO: 346 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPKG

SEQ ID NO: 347 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
KPGPPGPPGPPGPPGPPGPPG

SEQ ID NO: 348 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PKGPPGPPGPPGPPGPPGPPG

SEQ ID NO: 349 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGKPGPPGPPGPPGPPGPPG

SEQ ID NO: 350 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPKGPPGPPGPPGPPGPPG

SEQ ID NO: 351 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGKPGPPGPPGPPGPPG

SEQ ID NO: 352 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGKPGPPGPPGPPG

SEQ ID NO: 353 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPKGPPGPPGPPG

SEQ ID NO: 354 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGKPGPPGPPG

SEQ ID NO: 355 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPKGPPGPPG

SEQ ID NO: 356 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGKPGPPG

SEQ ID NO: 357 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPKGPPG

SEQ ID NO: 358 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGKPG

SEQ ID NO: 359 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPKG

SEQ ID NO: 360 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
KPGPPGPPGPPGPPGPPGPPG

SEQ ID NO: 361 - protein, 21 aa, synthetic construct
  REGION 1..21: note = Synthetic
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PKGPPGPPGPPGPPGPPGPPG

SEQ ID NO: 362 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGKPGPPGPPGPPGPPGPPG

SEQ ID NO: 363 - protein, 21 aa, synthetic construct
  REGION 1..21: note = Synthetic
  MOD_RES 2: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPKGPPGPPGPPGPPGPPG

SEQ ID NO: 364 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGKPGPPGPPGPPGPPG

SEQ ID NO: 365 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPKGPPGPPGPPGPPG

SEQ ID NO: 366 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGKPGPPGPPGPPG

SEQ ID NO: 367 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPKGPPGPPGPPG

SEQ ID NO: 368 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGKPGPPGPPG

SEQ ID NO: 369 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 17: note = Chloroproline (Clp)
  MOD_RES 20: note = Chloroproline (Clp)
PPGPPGPPGPPGPKGPPGPPG

SEQ ID NO: 370 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGKPGPPG

SEQ ID NO: 371 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 20: note = Chloroproline (Clp)
PPGPPGPPGPPGPPGPKGPPG

SEQ ID NO: 372 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)
PPGPPGPPGPPGPPGPPGKPG

SEQ ID NO: 373 - protein, 21 aa, synthetic construct
  REGION 1..21: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
PPGPPGPPGPPGPPGPPGPKG

SEQ ID NO: 374 - protein, 21 aa, synthetic construct
  REGION 1..21: note = Synthetic
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
KPGPPGPPGPPGPPGPPGPPG

SEQ ID NO: 375 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PKGPPGPPGPPGPPGPPGPPG

SEQ ID NO: 376 - protein, 21 aa, synthetic construct
  REGION 1..21: note = Synthetic
  MOD_RES 1: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGKPGPPGPPGPPGPPGPPG

SEQ ID NO: 377 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPKGPPGPPGPPGPPGPPG

SEQ ID NO: 378 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGKPGPPGPPGPPGPPG

SEQ ID NO: 379 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPKGPPGPPGPPGPPG

SEQ ID NO: 380 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGKPGPPGPPGPPG

SEQ ID NO: 381 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPKGPPGPPGPPG

SEQ ID NO: 382 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGKPGPPGPPG

SEQ ID NO: 383 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 17: note = Hydroxyproline (Hyp)
  MOD_RES 20: note = Hydroxyproline (Hyp)
PPGPPGPPGPPGPKGPPGPPG

SEQ ID NO: 384 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGKPGPPG

SEQ ID NO: 385 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 20: note = Hydroxyproline (Hyp)
PPGPPGPPGPPGPPGPKGPPG

SEQ ID NO: 386 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGKPG

SEQ ID NO: 387 - protein, 21 aa, synthetic construct
  REGION 1..21: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGPKG

SEQ ID NO: 388 - protein, 20 aa, synthetic construct
  REGION 1..20: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 16: note = Fluoroproline (Flp)
  MOD_RES 19: note = Fluoroproline (Flp)
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 18: note = Hydroxyproline (Hyp)
PPGPPGPPGPPGPPGPGPPG

SEQ ID NO: 389-395 - skipped (7 entries)

SEQ ID NO: 396 - protein, 30 aa, synthetic construct
  REGION 1..30: note = Synthetic
  MOD_RES 1: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
  MOD_RES 23: note = Hydroxyproline (Hyp)
  MOD_RES 26: note = Hydroxyproline (Hyp)
  MOD_RES 29: note = Hydroxyproline (Hyp)
PPGPPGPPGPPGPPGPPGPPGPPGPPGPPG

SEQ ID NO: 397 - protein, 21 aa, synthetic construct
  REGION 1..21: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
GGGPPGPPGPPGPPGPPGPPG

SEQ ID NO: 398 - protein, 21 aa, synthetic construct
  REGION 1..21: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
GGGPPGPPGPPGPPGPPGPPG

SEQ ID NO: 399 - protein, 24 aa, synthetic construct
  REGION 1..24: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
  MOD_RES 23: note = Hydroxyproline (Hyp)
GGGPPGPPGPPGPPGPPGPPGPPG

SEQ ID NO: 400 - protein, 24 aa, synthetic construct
  REGION 1..24: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
  MOD_RES 23: note = Fluoroproline (Flp)
GGGPPGPPGPPGPPGPPGPPGPPG

SEQ ID NO: 401 - protein, 27 aa, synthetic construct
  REGION 1..27: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
  MOD_RES 23: note = Hydroxyproline (Hyp)
  MOD_RES 26: note = Hydroxyproline (Hyp)
GGGPPGPPGPPGPPGPPGPPGPPGPPG

SEQ ID NO: 402 - protein, 27 aa, synthetic construct
  REGION 1..27: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
  MOD_RES 23: note = Fluoroproline (Flp)
  MOD_RES 26: note = Fluoroproline (Flp)
GGGPPGPPGPPGPPGPPGPPGPPGPPG

SEQ ID NO: 403 - protein, 30 aa, synthetic construct
  REGION 1..30: note = Synthetic
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
  MOD_RES 23: note = Hydroxyproline (Hyp)
  MOD_RES 26: note = Hydroxyproline (Hyp)
  MOD_RES 29: note = Hydroxyproline (Hyp)
GGGPPGPPGPPGPPGPPGPPGPPGPPGPPG

SEQ ID NO: 404 - protein, 30 aa, synthetic construct; identical to SEQ ID NO: 403
  REGION 1..30: note = Synthetic
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
  MOD_RES 23: note = Fluoroproline (Flp)
  MOD_RES 26: note = Fluoroproline (Flp)
  MOD_RES 29: note = Fluoroproline (Flp)

SEQ ID NO: 405 - protein, 19 aa, synthetic construct
  REGION 1..19: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
PPGPPGPPGPPGPPGPPGY

SEQ ID NO: 406 - protein, 19 aa, synthetic construct
  REGION 1..19: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
PPGPPGPPGPPGPPGPPGY

SEQ ID NO: 407 - protein, 22 aa, synthetic construct
  REGION 1..22: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
PPGPPGPPGPPGPPGPPGPPGY

SEQ ID NO: 408 - protein, 22 aa, synthetic construct
  REGION 1..22: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
PPGPPGPPGPPGPPGPPGPPGY

SEQ ID NO: 409 - protein, 25 aa, synthetic construct
  REGION 1..25: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)
  MOD_RES 23: note = Hydroxyproline (Hyp)
PPGPPGPPGPPGPPGPPGPPGPPGY

SEQ ID NO: 410 - protein, 25 aa, synthetic construct
  REGION 1..25: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)
  MOD_RES 23: note = Fluoroproline (Flp)
PPGPPGPPGPPGPPGPPGPPGPPGY

SEQ ID NO: 411 - protein, 10 aa, synthetic construct
  REGION 1..10: note = Synthetic
  MOD_RES 3: note = Hydroxyproline (Hyp)
  MOD_RES 6: note = Hydroxyproline (Hyp)
  MOD_RES 9: note = Hydroxyproline (Hyp)
CPPGPPGPPG

SEQ ID NO: 412 - protein, 10 aa, synthetic construct
  REGION 1..10: note = Synthetic
  MOD_RES 3: note = Fluoroproline (Flp)
  MOD_RES 6: note = Fluoroproline (Flp)
  MOD_RES 9: note = Fluoroproline (Flp)
CPPGPPGPPG

SEQ ID NO: 413 - protein, 16 aa, synthetic construct
  REGION 1..16: note = Synthetic
  MOD_RES 3: note = Hydroxyproline (Hyp)
  MOD_RES 6: note = Hydroxyproline (Hyp)
  MOD_RES 9: note = Hydroxyproline (Hyp)
  MOD_RES 12: note = Hydroxyproline (Hyp)
  MOD_RES 15: note = Hydroxyproline (Hyp)
CPPGPPGPPGPPGPPG

SEQ ID NO: 414 - protein, 16 aa, synthetic construct
  REGION 1..16: note = Synthetic
  MOD_RES 3: note = Fluoroproline (Flp)
  MOD_RES 6: note = Fluoroproline (Flp)
  MOD_RES 9: note = Fluoroproline (Flp)
  MOD_RES 12: note = Fluoroproline (Flp)
  MOD_RES 15: note = Fluoroproline (Flp)
CPPGPPGPPGPPGPPG

SEQ ID NO: 415 - protein, 22 aa, synthetic construct
  REGION 1..22: note = Synthetic
  MOD_RES 3: note = Hydroxyproline (Hyp)
  MOD_RES 6: note = Hydroxyproline (Hyp)
  MOD_RES 9: note = Hydroxyproline (Hyp)
  MOD_RES 12: note = Hydroxyproline (Hyp)
  MOD_RES 15: note = Hydroxyproline (Hyp)
  MOD_RES 18: note = Hydroxyproline (Hyp)
  MOD_RES 21: note = Hydroxyproline (Hyp)
CPPGPPGPPGPPGPPGPPGPPG

SEQ ID NO: 416 - protein, 22 aa, synthetic construct
  REGION 1..22: note = Synthetic
  MOD_RES 3: note = Fluoroproline (Flp)
  MOD_RES 6: note = Fluoroproline (Flp)
  MOD_RES 9: note = Fluoroproline (Flp)
  MOD_RES 12: note = Fluoroproline (Flp)
  MOD_RES 15: note = Fluoroproline (Flp)
  MOD_RES 18: note = Fluoroproline (Flp)
  MOD_RES 21: note = Fluoroproline (Flp)
CPPGPPGPPGPPGPPGPPGPPG

SEQ ID NO: 417 - protein, 60 aa, synthetic construct
  REGION 1..60: note = Synthetic
  VARIANT 1: note = X is proline, 4S-hydroxyproline, fluoroproline, chloroproline, lysine, cysteine or methionine
  VARIANT 4: note = X is proline, 4S-hydroxyproline, fluoroproline, chloroproline, lysine, cysteine or methionine
  VARIANT 7: note = X is proline, 4S-hydroxyproline, fluoroproline, chloroproline, lysine, cysteine or methionine
  VARIANT 10: note = X is proline, 4S-hydroxyproline, fluoroproline, chloroproline, lysine, cysteine or methionine
  VARIANT 13: note = X is proline, 4S-hydroxyproline, fluoroproline, chloroproline, lysine, cysteine or methionine
  VARIANT 16: note = X is proline, 4S-hydroxyproline, fluoroproline, chloroproline, lysine, cysteine or methionine
  VARIANT 19: note = X is proline, 4S-hydroxyproline, fluoroproline, chloroproline, lysine, cysteine or methionine
  VARIANT 22: note = X is proline, 4S-hydroxyproline, fluoroproline, chloroproline, lysine, cysteine or methionine
  VARIANT 25: note = X is proline, 4S-hydroxyproline, fluoroproline, chloroproline, lysine, cysteine or methionine
  VARIANT 28: note = X is proline, 4S-hydroxyproline, fluoroproline, chloroproline, lysine, cysteine or methionine
  VARIANT 31: note = X is proline, 4S-hydroxyproline, fluoroproline, chloroproline, lysine, cysteine or methionine
  VARIANT 34: note = X is proline, 4S-hydroxyproline, fluoroproline, chloroproline, lysine, cysteine or methionine
  VARIANT 37: note = X is proline, 4S-hydroxyproline, fluoroproline, chloroproline, lysine, cysteine or methionine
  VARIANT 40: note = X is proline, 4S-hydroxyproline, fluoroproline, chloroproline, lysine, cysteine or methionine
  VARIANT 43: note = X is proline, 4S-hydroxyproline, fluoroproline, chloroproline, lysine, cysteine or methionine
  VARIANT 46: note = X is proline, 4S-hydroxyproline, fluoroproline, chloroproline, lysine, cysteine or methionine
  VARIANT 49: note = X is proline, 4S-hydroxyproline, fluoroproline, chloroproline, lysine, cysteine or methionine
  VARIANT 52: note = X is proline, 4S-hydroxyproline, fluoroproline, chloroproline, lysine, cysteine or methionine
  VARIANT 55: note = X is proline, 4S-hydroxyproline, fluoroproline, chloroproline, lysine, cysteine or methionine
  VARIANT 58: note = X is proline, 4S-hydroxyproline, fluoroproline, chloroproline, lysine, cysteine or methionine
  VARIANT 2: note = X is proline, 4R-hydroxyproline, fluoroproline, chloroproline, lysine, cysteine or methionine
  VARIANT 5: note = X is proline, 4R-hydroxyproline, fluoroproline, chloroproline, lysine, cysteine or methionine
  VARIANT 8: note = X is proline, 4R-hydroxyproline, fluoroproline, chloroproline, lysine, cysteine or methionine
  VARIANT 11: note = X is proline, 4R-hydroxyproline, fluoroproline, chloroproline, lysine, cysteine or methionine
  VARIANT 14: note = X is proline, 4R-hydroxyproline, fluoroproline, chloroproline, lysine, cysteine or methionine
  VARIANT 17: note = X is proline, 4R-hydroxyproline, fluoroproline, chloroproline, lysine, cysteine or methionine
  VARIANT 20: note = X is proline, 4R-hydroxyproline, fluoroproline, chloroproline, lysine, cysteine or methionine
  VARIANT 23: note = X is proline, 4R-hydroxyproline, fluoroproline, chloroproline, lysine, cysteine or methionine
  VARIANT 26: note = X is proline, 4R-hydroxyproline, fluoroproline, chloroproline, lysine, cysteine or methionine
  VARIANT 29: note = X is proline, 4R-hydroxyproline, fluoroproline, chloroproline, lysine, cysteine or methionine
  VARIANT 32: note = X is proline, 4R-hydroxyproline, fluoroproline, chloroproline, lysine, cysteine or methionine
  VARIANT 35: note = X is proline, 4R-hydroxyproline, fluoroproline, chloroproline, lysine, cysteine or methionine
  VARIANT 38: note = X is proline, 4R-hydroxyproline, fluoroproline, chloroproline, lysine, cysteine or methionine
  VARIANT 41: note = X is proline, 4R-hydroxyproline, fluoroproline, chloroproline, lysine, cysteine or methionine
  VARIANT 44: note = X is proline, 4R-hydroxyproline, fluoroproline, chloroproline, lysine, cysteine or methionine
  VARIANT 47: note = X is proline, 4R-hydroxyproline, fluoroproline, chloroproline, lysine, cysteine or methionine
  VARIANT 50: note = X is proline, 4R-hydroxyproline, fluoroproline, chloroproline, lysine, cysteine or methionine
  VARIANT 53: note = X is proline, 4R-hydroxyproline, fluoroproline, chloroproline, lysine, cysteine or methionine
  VARIANT 56: note = X is proline, 4R-hydroxyproline, fluoroproline, chloroproline, lysine, cysteine or methionine
  VARIANT 59: note = X is proline, 4R-hydroxyproline, fluoroproline, chloroproline, lysine, cysteine or methionine
  VARIANT 4..60: note = "XXG" may or may not be present
XXGXXGXXGXXGXXGXXGXXGXXGXXGXXGXXGXXGXXGXXGXXGXXGXXGXXGXXGXXG

SEQ ID NO: 418 - protein, 21 aa, synthetic construct
  REGION 1..21: note = Synthetic
PAGPAGPAGPAGPAGPAGPAG

SEQ ID NO: 419 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PAGPAGPAGPAGPAGPAGPAG

SEQ ID NO: 420 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PAGPAGPAGPAGPAGPAGPAG

SEQ ID NO: 421 - protein, 21 aa, synthetic construct
  REGION 1..21: note = Synthetic
PGEPGEPGEPGEPGEPGEPGE

SEQ ID NO: 422 - protein, 21 aa, synthetic construct
  REGION 1..21: note = Synthetic
PGQPGQPGQPGQPGQPGQPGQ

SEQ ID NO: 423 - protein, 21 aa, synthetic construct
  REGION 1..21: note = Synthetic
PGPPGPPGPPGPPGPPGPPGP

SEQ ID NO: 424 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGEPGEPGEPGEPGEPGEPGE

SEQ ID NO: 425 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGQPGQPGQPGQPGQPGQPGQ

SEQ ID NO: 426 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGEPGEPGEPGEPGEPGEPGE

SEQ ID NO: 427 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGQPGQPGQPGQPGQPGQPGQ

SEQ ID NO: 428 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGPPGPPGPPGPPGPPGPPGP

SEQ ID NO: 429 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGQPGQPGQPGQPGQPGQPGQ

SEQ ID NO: 430 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGEPGEPGEPGEPGEPGEPGE

SEQ ID NO: 431 - protein, 21 aa, synthetic construct
  REGION 1..21: note = Synthetic
PGDPGDPGDPGDPGDPGDPGD

SEQ ID NO: 432 - protein, 21 aa, synthetic construct
  REGION 1..21: note = Synthetic
PGNPGNPGNPGNPGNPGNPGN

SEQ ID NO: 433 - protein, 21 aa, synthetic construct
  REGION 1..21: note = Synthetic
KGQKGQKGQKGQKGQKGQKGQ

SEQ ID NO: 434 - protein, 21 aa, synthetic construct
  REGION 1..21: note = Synthetic
KGEKGEKGEKGEKGEKGEKGE

SEQ ID NO: 435 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PAGPAGPAGPAGPAGPAGPAG

SEQ ID NO: 436 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PAGPAGPAGPAGPAGPAGPAG

SEQ ID NO: 437 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGEPGEPGEPGEPGEPGEPGE

SEQ ID NO: 438 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGQPGQPGQPGQPGQPGQPGQ

SEQ ID NO: 439 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGEPGEPGEPGEPGEPGEPGE

SEQ ID NO: 440 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGQPGQPGQPGQPGQPGQPGQ

SEQ ID NO: 441 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGDPGDPGDPGDPGDPGDPGD

SEQ ID NO: 442 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGNPGNPGNPGNPGNPGNPGN

SEQ ID NO: 443 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGDPGDPGDPGDPGDPGDPGD

SEQ ID NO: 444 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGNPGNPGNPGNPGNPGNPGN

SEQ ID NO: 445 - protein, 21 aa, synthetic construct
  REGION 1..21: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)
PGPPGPPGPPGPPGPPGPPGP

SEQ ID NO: 446 - protein, 21 aa, synthetic construct
  REGION 1..21: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
PGPPGPPGPPGPPGPPGPPGP

SEQ ID NO: 447 - protein, 21 aa, synthetic construct
  REGION 1..21: note = Synthetic
  MOD_RES 3: note = Hydroxyproline (Hyp)
  MOD_RES 6: note = Hydroxyproline (Hyp)
  MOD_RES 9: note = Hydroxyproline (Hyp)
  MOD_RES 12: note = Hydroxyproline (Hyp)
  MOD_RES 15: note = Hydroxyproline (Hyp)
  MOD_RES 18: note = Hydroxyproline (Hyp)
  MOD_RES 21: note = Hydroxyproline (Hyp)
PGPPGPPGPPGPPGPPGPPGP

SEQ ID NO: 448 - protein, 21 aa, synthetic construct
  REGION 1..21: note = Synthetic
  MOD_RES 1: note = fluoroproline (Flp)
  MOD_RES 4: note = fluoroproline (Flp)
  MOD_RES 7: note = fluoroproline (Flp)
  MOD_RES 10: note = fluoroproline (Flp)
  MOD_RES 13: note = fluoroproline (Flp)
  MOD_RES 16: note = fluoroproline (Flp)
  MOD_RES 19: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
PGPPGPPGPPGPPGPPGPPGP

SEQ ID NO: 449 - protein, 21 aa, synthetic construct
  REGION 1..21: note = Synthetic
  MOD_RES 1: note = Hydroxyproline (Hyp)
  MOD_RES 3..4: note = Hydroxyproline (Hyp)
  MOD_RES 6..7: note = Hydroxyproline (Hyp)
  MOD_RES 9..10: note = Hydroxyproline (Hyp)
  MOD_RES 12..13: note = Hydroxyproline (Hyp)
  MOD_RES 15..16: note = Hydroxyproline (Hyp)
  MOD_RES 18..19: note = Hydroxyproline (Hyp)
  MOD_RES 21: note = Hydroxyproline (Hyp)
PGPPGPPGPPGPPGPPGPPGP

SEQ ID NO: 450 - protein, 21 aa, synthetic construct
  REGION 1..21: note = Synthetic
  MOD_RES 1: note = hydroxyproline (Hyp)
  MOD_RES 4: note = hydroxyproline (Hyp)
  MOD_RES 7: note = hydroxyproline (Hyp)
  MOD_RES 10: note = hydroxyproline (Hyp)
  MOD_RES 13: note = hydroxyproline (Hyp)
  MOD_RES 16: note = hydroxyproline (Hyp)
  MOD_RES 19: note = hydroxyproline (Hyp)
  MOD_RES 3: note = fluoroproline (Flp)
  MOD_RES 6: note = fluoroproline (Flp)
  MOD_RES 9: note = fluoroproline (Flp)
  MOD_RES 12: note = fluoroproline (Flp)
  MOD_RES 15: note = fluoroproline (Flp)
  MOD_RES 18: note = fluoroproline (Flp)
  MOD_RES 21: note = fluoroproline (Flp)
PGPPGPPGPPGPPGPPGPPGP

SEQ ID NO: 451 - protein, 21 aa, synthetic construct
  REGION 1..21: note = Synthetic
  MOD_RES 3: note = Fluoroproline (Flp)
  MOD_RES 6: note = Fluoroproline (Flp)
  MOD_RES 9: note = Fluoroproline (Flp)
  MOD_RES 12: note = Fluoroproline (Flp)
  MOD_RES 15: note = Fluoroproline (Flp)
  MOD_RES 18: note = Fluoroproline (Flp)
  MOD_RES 21: note = Fluoroproline (Flp)
PGPPGPPGPPGPPGPPGPPGP

SEQ ID NO: 452 - protein, 21 aa, synthetic construct
  REGION 1..21: note = Synthetic
  MOD_RES 1: note = Fluoroproline (Flp)
  MOD_RES 3..4: note = Fluoroproline (Flp)
  MOD_RES 6..7: note = Fluoroproline (Flp)
  MOD_RES 9..10: note = Fluoroproline (Flp)
  MOD_RES 12..13: note = Fluoroproline (Flp)
  MOD_RES 15..16: note = Fluoroproline (Flp)
  MOD_RES 18..19: note = Fluoroproline (Flp)
  MOD_RES 21: note = Fluoroproline (Flp)
PGPPGPPGPPGPPGPPGPPGP

SEQ ID NO: 453 - protein, 22 aa, synthetic construct
  REGION 1..22: note = Synthetic
CPPGPPGPPGPPGPPGPPGPPG

SEQ ID NO: 454 - protein, 22 aa, synthetic construct
  REGION 1..22: note = Synthetic
PPGPPGPPGPPGPPGPPGPPGC

SEQ ID NO: 455 - protein, 23 aa, synthetic construct
  REGION 1..23: note = Synthetic
CPPGPPGPPGPPGPPGPPGPPGC

SEQ ID NO: 456 - protein, 22 aa, synthetic construct
  REGION 1..22: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
CPPGPPGPPGPPGPPGPPGPPG

SEQ ID NO: 457 - protein, 22 aa, synthetic construct
  REGION 1..22: note = Synthetic
  MOD_RES 1: note = fluoroproline (Flp)
  MOD_RES 4: note = fluoroproline (Flp)
  MOD_RES 7: note = fluoroproline (Flp)
  MOD_RES 10: note = fluoroproline (Flp)
  MOD_RES 13: note = fluoroproline (Flp)
  MOD_RES 16: note = fluoroproline (Flp)
  MOD_RES 19: note = fluoroproline (Flp)
  MOD_RES 2: note = hydroxyproline (Hyp)
  MOD_RES 5: note = hydroxyproline (Hyp)
  MOD_RES 8: note = hydroxyproline (Hyp)
  MOD_RES 11: note = hydroxyproline (Hyp)
  MOD_RES 14: note = hydroxyproline (Hyp)
  MOD_RES 17: note = hydroxyproline (Hyp)
  MOD_RES 20: note = hydroxyproline (Hyp)
PPGPPGPPGPPGPPGPPGPPGC

SEQ ID NO: 458 - protein, 23 aa, synthetic construct
  REGION 1..23: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
CPPGPPGPPGPPGPPGPPGPPGC

SEQ ID NO: 459 - protein, 22 aa, synthetic construct
  REGION 1..22: note = Synthetic
MPPGPPGPPGPPGPPGPPGPPG

SEQ ID NO: 460 - protein, 22 aa, synthetic construct
  REGION 1..22: note = Synthetic
PPGPPGPPGPPGPPGPPGPPGM

SEQ ID NO: 461 - protein, 23 aa, synthetic construct
  REGION 1..23: note = Synthetic
MPPGPPGPPGPPGPPGPPGPPGM

SEQ ID NO: 462 - protein, 22 aa, synthetic construct
  REGION 1..22: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
MPPGPPGPPGPPGPPGPPGPPG

SEQ ID NO: 463 - protein, 22 aa, synthetic construct
  REGION 1..22: note = Synthetic
  MOD_RES 1: note = fluoroproline (Flp)
  MOD_RES 4: note = fluoroproline (Flp)
  MOD_RES 7: note = fluoroproline (Flp)
  MOD_RES 10: note = fluoroproline (Flp)
  MOD_RES 13: note = fluoroproline (Flp)
  MOD_RES 16: note = fluoroproline (Flp)
  MOD_RES 19: note = fluoroproline (Flp)
  MOD_RES 2: note = hydroxyproline (Hyp)
  MOD_RES 5: note = hydroxyproline (Hyp)
  MOD_RES 8: note = hydroxyproline (Hyp)
  MOD_RES 11: note = hydroxyproline (Hyp)
  MOD_RES 14: note = hydroxyproline (Hyp)
  MOD_RES 17: note = hydroxyproline (Hyp)
  MOD_RES 20: note = hydroxyproline (Hyp)
PPGPPGPPGPPGPPGPPGPPGM

SEQ ID NO: 464 - protein, 23 aa, synthetic construct
  REGION 1..23: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
MPPGPPGPPGPPGPPGPPGPPGM

SEQ ID NO: 465 - protein, 22 aa, synthetic construct
  REGION 1..22: note = Synthetic
KPPGPPGPPGPPGPPGPPGPPG

SEQ ID NO: 466 - protein, 22 aa, synthetic construct
  REGION 1..22: note = Synthetic
PPGPPGPPGPPGPPGPPGPPGK

SEQ ID NO: 467 - protein, 23 aa, synthetic construct
  REGION 1..23: note = Synthetic
KPPGPPGPPGPPGPPGPPGPPGK

SEQ ID NO: 468 - protein, 22 aa, synthetic construct
  REGION 1..22: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
KPPGPPGPPGPPGPPGPPGPPG

SEQ ID NO: 469 - protein, 22 aa, synthetic construct
  REGION 1..22: note = Synthetic
  MOD_RES 1: note = fluoroproline (Flp)
  MOD_RES 4: note = fluoroproline (Flp)
  MOD_RES 7: note = fluoroproline (Flp)
  MOD_RES 10: note = fluoroproline (Flp)
  MOD_RES 13: note = fluoroproline (Flp)
  MOD_RES 16: note = fluoroproline (Flp)
  MOD_RES 19: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
PPGPPGPPGPPGPPGPPGPPGK

SEQ ID NO: 470 - protein, 23 aa, synthetic construct
  REGION 1..23: note = Synthetic
  MOD_RES 2: note = fluoroproline (Flp)
  MOD_RES 5: note = fluoroproline (Flp)
  MOD_RES 8: note = fluoroproline (Flp)
  MOD_RES 11: note = fluoroproline (Flp)
  MOD_RES 14: note = fluoroproline (Flp)
  MOD_RES 17: note = fluoroproline (Flp)
  MOD_RES 20: note = fluoroproline (Flp)
  MOD_RES 3: note = hydroxyproline (Hyp)
  MOD_RES 6: note = hydroxyproline (Hyp)
  MOD_RES 9: note = hydroxyproline (Hyp)
  MOD_RES 12: note = hydroxyproline (Hyp)
  MOD_RES 15: note = hydroxyproline (Hyp)
  MOD_RES 18: note = hydroxyproline (Hyp)
  MOD_RES 21: note = hydroxyproline (Hyp)
KPPGPPGPPGPPGPPGPPGPPGK

SEQ ID NO: 471 - protein, 11 aa, synthetic construct
  REGION 1..11: note = Synthetic
RPKPQQFFGLM

SEQ ID NO: 472 - protein, 32 aa, synthetic construct
  REGION 1..32: note = Synthetic
PPGPPGPPGPPGPPGPPGPPGRPKPQQFFGLM

SEQ ID NO: 473 - protein, 32 aa, synthetic construct; identical to SEQ ID NO: 472
  REGION 1..32: note = Synthetic
  MOD_RES 1: note = Hydroxyproline (Hyp)
  MOD_RES 4: note = Hydroxyproline (Hyp)
  MOD_RES 7: note = Hydroxyproline (Hyp)
  MOD_RES 10: note = Hydroxyproline (Hyp)
  MOD_RES 13: note = Hydroxyproline (Hyp)
  MOD_RES 16: note = Hydroxyproline (Hyp)
  MOD_RES 19: note = Hydroxyproline (Hyp)

SEQ ID NO: 474 - protein, 32 aa, synthetic construct; identical to SEQ ID NO: 472
  REGION 1..32: note = Synthetic
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)

SEQ ID NO: 475 - protein, 32 aa, synthetic construct; identical to SEQ ID NO: 472
  REGION 1..32: note = Synthetic
  MOD_RES 1: note = Fluoroproline (Flp)
  MOD_RES 4: note = Fluoroproline (Flp)
  MOD_RES 7: note = Fluoroproline (Flp)
  MOD_RES 10: note = Fluoroproline (Flp)
  MOD_RES 13: note = Fluoroproline (Flp)
  MOD_RES 16: note = Fluoroproline (Flp)
  MOD_RES 19: note = Fluoroproline (Flp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)

SEQ ID NO: 476 - protein, 32 aa, synthetic construct; identical to SEQ ID NO: 472
  REGION 1..32: note = Synthetic
  MOD_RES 1: note = Chloroproline (Clp)
  MOD_RES 4: note = Chloroproline (Clp)
  MOD_RES 7: note = Chloroproline (Clp)
  MOD_RES 10: note = Chloroproline (Clp)
  MOD_RES 13: note = Chloroproline (Clp)
  MOD_RES 16: note = Chloroproline (Clp)
  MOD_RES 19: note = Chloroproline (Clp)
  MOD_RES 2: note = Hydroxyproline (Hyp)
  MOD_RES 5: note = Hydroxyproline (Hyp)
  MOD_RES 8: note = Hydroxyproline (Hyp)
  MOD_RES 11: note = Hydroxyproline (Hyp)
  MOD_RES 14: note = Hydroxyproline (Hyp)
  MOD_RES 17: note = Hydroxyproline (Hyp)
  MOD_RES 20: note = Hydroxyproline (Hyp)

SEQ ID NO: 477 - protein, 32 aa, synthetic construct; identical to SEQ ID NO: 472
  REGION 1..32: note = Synthetic
  MOD_RES 2: note = Fluoroproline (Flp)
  MOD_RES 5: note = Fluoroproline (Flp)
  MOD_RES 8: note = Fluoroproline (Flp)
  MOD_RES 11: note = Fluoroproline (Flp)
  MOD_RES 14: note = Fluoroproline (Flp)
  MOD_RES 17: note = Fluoroproline (Flp)
  MOD_RES 20: note = Fluoroproline (Flp)

SEQ ID NO: 478 - protein, 32 aa, synthetic construct; identical to SEQ ID NO: 472
  REGION 1..32: note = Synthetic
  MOD_RES 2: note = Chloroproline (Clp)
  MOD_RES 5: note = Chloroproline (Clp)
  MOD_RES 8: note = Chloroproline (Clp)
  MOD_RES 11: note = Chloroproline (Clp)
  MOD_RES 14: note = Chloroproline (Clp)
  MOD_RES 17: note = Chloroproline (Clp)
  MOD_RES 20: note = Chloroproline (Clp)

SEQ ID NO: 479 - protein, 30 aa, synthetic construct; identical to SEQ ID NO: 396
  REGION 1..30: note = Synthetic

(19) United States
(12) Patent Application Publication
(10) Pub. No.: US 2026/0115261 A1
(43) Pub. Date: Apr. 30, 2026

(54) COLLAGEN PEPTIDE-BASED MEDICAMENT COMPOSITIONS AND USES THEREOF

(71) Applicant: SUSTAIN HOLDINGS, LLC, Stuart, FL (US)

(72) Inventors: Richard E. SCHLUMPF, Stuart, FL (US); Brian J. DEL BUONO, Stuart, FL (US); Robert O. BARATTA, Stuart, FL (US); David J. CALKINS, Stuart, FL (US)

(21) Appl. No.: 18/932,246

(22) Filed: Oct. 30, 2024

Related U.S. Application Data
(60) Provisional application No. 63/594,245, filed on Oct. 30, 2023.

(51) Int. Cl. A61K 38/39; A61K 47/64; A61P 9/00; A61P 25/00

(52) U.S. Cl. CPC A61K 38/39; A61K 47/64; A61P 9/00; A61P 25/00

(57) ABSTRACT

The present invention is in the fields of medicinal chemistry, biotechnology and pharmaceuticals. The invention provides compositions comprising one or more collagen mimetic peptides, optionally attached to one or more therapeutic compounds or one or more imaging compounds, methods for use of such compositions in treating, preventing, ameliorating, curing and/or diagnosing certain diseases and physical disorders in humans and veterinary animals, particularly nervous system diseases, disorders and injuries and cardiovascular system diseases, disorders and injuries. The invention also provides the use of such compositions in treating, preventing, ameliorating, curing and/or diagnosing diseases, disorders and conditions in a variety of other tissues, organs and organ systems. The invention also provides medical devices comprising one or more such compositions of the invention.

### CROSS-REFERENCE TO RELATED APPLICATIONS AND INCORPORATION BY REFERENCE

The present application claims the benefit of the filing date of U.S. Provisional Patent Application No. 63/594,245, filed Oct. 30, 2023, entitled “Collagen Peptide-Based Medicament Compositions and Uses Thereof” and naming as inventors Richard E. Schlumpf, Brian J. Del Buono, Robert O. Baratta, and David J. Calkins, and the entirety of which is incorporated herein by reference.

### STATEMENT REGARDING FEDERALLY SPONSORED RESEARCH AND DEVELOPMENT

Not applicable.

### NAMES OF THE PARTIES TO A JOINT RESEARCH AGREEMENT

Not applicable.

### REFERENCE TO SEQUENCE LISTING SUBMITTED ELECTRONICALLY

The contents of the electronic sequence listing (0123-0014US1_SL.xml; Size: 1830 KB; and Date of Creation Jan. 8, 2025) submitted herewith, is herein incorporated by reference in its entirety.

### BACKGROUND OF THE INVENTION

### Field of the Invention

The present invention is in the fields of medicinal chemistry, biotechnology, pharmaceuticals and medical devices, as well as the use of medicinal compounds and medical devices for the treatment, prevention and amelioration of diseases, disorders and physical ailments in humans and veterinary animals, particularly certain neurological diseases and disorders including those involving the central nervous system and the peripheral nervous system, as well as certain cardiovascular diseases and disorders.

### Background Art

Collagen is the most abundant protein in vertebrates, and is the fundamental structural protein for vertebrate tissues, occurring in virtually every tissue including skin and other epithelial tissues (including the lining of most luminal organs such as those of the gastrointestinal tract), tendons, bone, blood vessels, cartilage, ligaments and teeth. In humans, collagen makes up about a third of the total protein and about three-quarters of the dry weight of skin (see Shoulders, M. D., and Raines, R. T., Ann. Rev. Biochem. 78:929-958 (2009); Gelse, K., et al., Adv. Drug Deliv. Rev. 55:1531-1546 (2003)).

Collagen is a fibrous protein that is composed of a triple helix, which generally consists of two identical chains and a third chain that differs slightly in its chemical composition. Mammals produce at least 46 distinct collagen polypeptide chains that combine to form variants or “types” of collagen. To date, 28 types of collagen have been described. Collagen types are generally grouped according to their structural forms: fibrillar (types I, II, III, V and XI) which represent about 90% of all collagen protein found in mammals, and non-fibrillar (basement membrane or type IV, and other non-fibrillar collagen types with interrupted helix structures) see Id.). The five most common types of collagen, and their tissue distributions, are:
Type I: skin, tendon, organs, bone, vascular connective tissue;
Type II: cartilage;
Type III: reticular connective tissue, often associated with Type I collagen;
Type IV: basement membranes of epithelial tissues and certain solid tumors; and
Type V: hair, placenta, external cellular membranes.

In each of these variants, the polypeptide chains of collagen are composed of approximately 300 repeats of the amino acids proline (Pro), 4(R)-hydroxyproline (Hyp) and glycine (Gly), usually in the sequence X-Y-Gly, where X is often a Pro residue and Y is often a (Hyp) residue; in vertebrates, the typical repeat motif in collagen is ProProGly (see Hulmes, D. J. S., “Collagen Diversity, Synthesis and Assembly,” in: Collagen: Structure and Mechanics, P. Fratzl, Ed., New York: Springer, pp. 15-47 (2008)). Subsequently, in vivo, the hydroxylation of Pro residues is performed enzymatically after collagen biosynthesis but before the chains begin to form a triple helix. Thus, hydroxylation of at least one Pro residue in the ProProGly motif, typically forming ProHypGly, appears to be important for both the proper folding and stability of the collagen triple helix, both of which are key to the normal structure and function of collagen in vivo (see Shoulders, M. D., and Raines, R. T., Ann. Rev. Biochem. 78:929-958 (2009)). For example, the melting temperature of a triple helix of (ProHypGly)₁₀ (SEQ ID NO: 396) chains is 58° C., while that of a triple helix of (ProProGly)₁₀ (SEQ ID NO: 479) chains is only 24° C. (Sakakibara et al., Biochim. Biophys. Acta, 303:198-202 (1973)), and the rate at which (ProHypGly)₁₀ (SEQ ID NO: 396) chains fold into a triple helix is substantially greater than the corresponding rate for (ProProGly)₁₀ SEQ ID NO: 479) chains (Chopra and Ananthanarayanan, Proc. Natl. Acad. Sci. USA, 79:7180-7184 (1982)).

Type I collagen is the most abundant and best-studied collagen. In humans and most other animals it forms more than 90% of the organic mass of bone and is the major collagen of tendons, skin, ligaments, cornea, and many interstitial connective tissues with the exception of a very few such as hyaline cartilage, brain and the vitreous body. The collagen type I triple helix is usually formed as a heterotrimer by two identical α1 chains and one α2 chain. The triple helical fibers are, in vivo, primarily incorporated into composite fibrils containing other types of collagens, which as noted above vary depending upon tissue type and location (Fleischmajer, E. D. et al., J. Struct. Biol. 105: 162-169 (1990); Niyibizi, C. and Eyre, D. R., Connect. Tissue Res. 20: 247-250 (1989)). In most organs and notably in tendons and fascia, type I collagen provides tensile rigidity and in bone, it defines the biomechanical properties relating to load bearing, tensile strength and torsional stiffness.

In connective tissues (such as bone, tendon, cartilage, ligament, skin, blood vessels and teeth), individual collagen molecules are wound together in tight triple helices. These helices are organized into fibrils of great tensile strength (Jones & Miller, J. Mol. Biol., 218:209-219 (1991)) via cross-linking of individual triple helix fibers (Lodish, H. et al., “Collagen: The Fibrous Proteins of the Matrix”, in: Molecular Cell Biology, 4th ed., Section 22.3, New York: W. H. Freeman (2000)). Varying the arrangements and cross linking of the collagen fibrils enables vertebrates to support stress in one dimension (tendons), two dimensions (skin) or three dimensions (cartilage).

Collagens serve within the body to a considerable extent for the maintenance of the structural integrity of tissues and organs. In all parenchymal organs, collagens represent the major component of the interstitial matrix as well as the basement membranes, while in all connective tissues, particularly bone and cartilage, collagens provide the major functional backbone of the structures. Besides the biomechanical aspects, however, collagens are also involved in a variety of additional functions. For example, specific cell surface and intracellular receptors interact with collagens, and signaling by these receptors is involved in cellular adhesion, differentiation, growth and other cellular activities, as well as the survival of cells both in vivo and in vitro (Vogel, W. F., Eur. J. Dermatol. 11: 506-514 (2001); Gelse, K., et al., Adv. Drug Deliv. Rev. 55:1531-1546 (2003)). Collagens also are involved in the entrapment, local storage and delivery of growth factors and cytokines in a variety of tissues in which the collagens are found. Through these receptor interactions and storage and delivery functions, collagen plays a key role in organ development, wound healing and tissue repair (Chattopadhyay, S. and R. Raines, Biopolymers 101: 821-833 (2014); Yamaguchi, Y. et al., Nature 346: 281-284 (1990); Hay, E. D., J. Cell Biol. 91:205s-223s (1981); Bautista, C. M. et al., Metabolism 39: 96-100 (1990); Zhu, Y. et al., J. Cell Biol. 144: 1069-1080 (1998); Schlegel, K. A. et al., Biomaterials 25:5387-5393 (2004); Kumar, V. A., et al., Biomacromol. 15: 1484-1490 (2014)). These functions also qualify collagens as candidate transport vehicles for the delivery of therapeutic compounds (see, e.g., Chattopadhyay, S. et al., J. Tissue Eng. Regen. Med. 10:1012-1020 (2012); Schuppan, D. et al., Gastroenterol. 114: 139-152 (1998); Frenkel, S. R. et al., J. Bone Jt. Surg. 79-B: 831-836 (1997); Albu, M. G. et al., “Collagen-Based Drug Delivery Systems for Tissue Engineering”, in: Biomaterials Applications for Nanomedicine, Pignatello, R. (Ed.), ISBN: 978-953-307-661-4, DOI: 10.5772/22981, Rijeka, Croatia: InTech, available from: https://www.intechopen.com/books/biomaterials-applications-for-nanomedicine/collagen-based-drug-delivery-systems-for-tissue-engineering (2011)), and for use in wound healing by directly promoting tissue repair or regeneration (Wakitani, S. et al., J. Bone Jt. Surg. 71-B: 74-80 (1989); Kumar, V. A., et al., Biomacromol. 15: 1484-1490 (2014)). Collagen (more particularly, disrupted collagen) has also been implicated in tumor progression and metastasis in humans and other vertebrates (for a review of this issue, see Fang, M., et al., Tumor Biol. 35:2871-2882 (2014)).

Beyond intact collagen molecules, however, fragments of collagen may also have potential therapeutic uses, and indeed, may perform in a superior fashion relative to native collagen. For example, non-collagenous fragments of collagens IV, XV and XVIII have been shown to promote the growth of blood vessels and tumor cells, and to influence a variety of other cellular activities (Ortega, N. and Werb, Z., J. Cell Sci. 115: 4201-4214 (2002); Davis, G. E. et al., Am. J. Pathol. 156: 1489-1498 (2000); O'Reilly, M. S. et al., Cell 88: 277-285 (1997)). Analogously, as described in greater detail below, fragments or synthetic collagen mimetic peptides (CMPs) of collagen type I have been studied for their utility in treatment of diseases and medical disorders, both as active pharmaceutical ingredients (APIs) in their own right and in the delivery of a skin wound-healing agent (see U.S. Pat. Nos. 5,973,112, 7,122,521, 7,858,741, and U.S. Patent Publ. No. US 2007/0275897 A1, the disclosures of all of which are incorporated herein by reference in their entireties; see also e.g., Chattopadhyay, S. et al., J. Tissue Eng. Regen. Med. 10:1012-1020 (2012); Kumar, V. A. et al., Biomacromolecules 15:1484-1490 (2014)).

Collagen abnormalities are associated with a wide variety of human diseases, including diseases and disorders of the eye such as cataracts and glaucoma (Coudrillier, B., et al., PLoS ONE 10: e0131396 (2015); Huang, W. et al., Med. Sci. Monit. Basic Res. 19: 237-240 (2013); Dua, H. S., et al., Br. J. Ophthalmol. 98: 691-697 (2014)), arthritis, rheumatism, brittle bones, atherosclerosis and cirrhosis. Disruptions in collagen are also associated with certain human and veterinary diseases such as certain cancers (particularly carcinomas of the luminal organs, and certain sarcomas); see, e.g., Lauer, J. L., and Fields, G. B., “Collagen in Cancer”, in The Tumor Microenvironment, New York: Springer, pp. 477-507 (2010). Collagen is also critically important in wound healing and is known to be upregulated in areas of epithelial wounds where healing is taking place (see, e.g., U.S. Pat. Nos. 5,973,112 and 7,122,521, which are incorporated herein by reference in their entireties; see also Chattopadhyay, S., et al., J. Tissue Eng. Regen. Med. 10:1012-1020 (2012); Chattopadhyay, S., et al., Org. Biomol. Chem. 10:5892-5897 (2012); Kumar, V. A., et al., Biomacromol. 15: 1484-1490 (2014)), including in the skin and the cornea of the eye. Indeed, collagen, collagen fragments or certain mimetic peptides of natural collagen have been reported to show promise in treating certain wounds and diseases in humans and animals, particularly skin wounds (see, e.g., U.S. Pat. Nos. 5,973,112, 7,122,521, 7,858,741, and U.S. Patent Publ. No. US 2007/0275897 A1, all of which are incorporated herein by reference in their entireties; see also Kumar, V. A. et al., Biomacromolecules 15:1484-1490 (2014)). It is thought that these collagen fragments or collagen mimetic peptides may specifically target areas of collagen disruption associated with skin wounds by intercalating into the disrupted collagen and reforming the native collagen I triple helix (see, e.g., Chattopadhyay, S., et al., J. Tissue Eng. Regen. Med. 10:1012-1020 (2012); Chattopadhyay, S., et al., Org. Biomol. Chem. 10:5892-5897 (2012)). As a result, there have been attempts made to use collagen as a vehicle for delivering certain drugs, with varying degrees of success (see, e.g., B. An, et al., Adv. Drug Deliv. Rev. 97:69-84 (2016); V. Chak, et al., Intl. J. Pharm. Teaching and Practices 4:811 (2013)). Collagen mimetic peptides have also been used in a topical application to deliver a conjugated therapeutic compound, the neuropeptide known as Substance P, to areas of skin wounds; such CMP-Substance P conjugates have been shown to accelerate wound healing in a mouse skin model (Chattopadhyay, S., et al., J. Tissue Eng. Regen. Med. 10:1012-1020 (2012)). Certain extracellular matrix (ECM) components, including collagens, are also involved in maintaining proper structure and function of the nervous system, particularly the peripheral nervous system, and disruption of or damage to these ECM components often leads to nerve cell disorder and/or death (see, e.g., Koopmans G, Hasse B, Sinis N. The role of collagen in peripheral nerve repair (Chapter 19). International Review of Neurobiology. Volume 87: Academic Press, Elsevier; pp. 363-79 (2009); Gao X, et al., Rev. Neurosci. 24(4):443-53 (2013); Campbell I C et al., J. Biomech. Eng. 136(2):021005 (2014); Vecino E et al., J. Cytol. Histol. S3:007 (2015); Vecino E., and Kwok, J. C. F., “The Extracellular Matrix in the Nervous System: The Good and the Bad Aspects”, in Composition and Function of the Extracellular Matrix in the Human Body, F. Travascio, ed., Intech Open, ISBN 978-953-51-2416-0 (2016), accessed Nov. 8, 2019, at http://dx.doi.org/10.5772/62527).

Treatments for diseases/disorders are expensive, difficult to deliver with specificity, and may have deleterious effects at sites distal to the intended site of action. For example, many medicinal compositions, including antibiotics, small molecule therapeutics (e.g., anti-cancer compounds) and biologics (e.g., monoclonal antibody therapeutics) are administered parenterally in a non-targeted fashion and must diffuse or otherwise find their way to the site of the affliction before they are able to provide their therapeutic benefits. This “shotgun approach” to therapy necessarily requires higher dosing and can result in longer periods of therapy and reduced patient compliance than a therapeutic approach which would deliver therapeutic compounds and compositions in a more targeted fashion which would allow for controlled or programmable release at or near the site of the affliction in a human or veterinary animal. In particular, ocular diseases, disorders and physical conditions have often proven to be difficult to treat and/or remediate. For example, presbyopia is an ocular disorder that is frequently associated with aging (Lafosse, E. et al., Cont. Lens Ant. Eye 43(2): 103-114 (2020); Balgos, M. J. T. D et al., Taiwan J. Ophthalmol 8:121-140 (2018); Katz, J. A. et al., Clin. Ophthalmol. 15:2167-2178 (2021)) and is often treated via optical correction either mechanically (e.g., via eyeglasses or contact lenses) or via refractive surgery (e.g., conductive keratoplasty, LASIK/LASEK surgery, photorefractive keratectomy, or via lens implants). Myopia is an ocular disorder that is found in all ages including both children (Hou, W. et al., Eye Contact Lens 44(4):248-259 (2018); Lau, J. K. et al., Invest. Ophthalmol. 61(2):22 (2020); Tideman, J. W. L. et al., Acta Ophthalmol. 96:301-309 (2018)) and adults (Kim, H. K. et al., Int. J. Ophthalmol. 14(8):1231-1236 (2021); Wang, B. et al., PLoS ONE 12(4):e0175913 (2017); Pugazhendhi, S. et al., Clin. Ophthalmol. 14:853-873 (2020)) which is treated with corrective lenses and/or pharmaceutically, e.g., with atropine or pirenzepine (Gwiazda, J., Optom. Vis. Sci. 86(6):624-628 (2009)) and other pharmacotherapeutic approaches (Wang, W.-Y. et al, Biomed. Pharmacother. 133:111092 (2021)). Particularly in children and the elderly, patient compliance with such corrective measures is often incomplete due to inconvenience, discomfort or the like, leading to continued, and in some cases worsening, loss of vision.

Previous work from some of the present inventors has demonstrated and/or suggested that certain CMP-containing formulations may be useful in treating certain front-of-eye ocular conditions such as dry eye disease and other corneal diseases, disorders and injuries (see, e.g., U.S. Pat. No. 10,632,168; Baratta, R. O. et al., Baratta et al., Front. Pharmacol. 12:705623 (2021); Baratta, R. O. et al., Surv. Ophthalmol. 67:60-67 (2022)), and certain back-of-eye disorders and conditions such as glaucoma, macular degeneration, optic neuropathy and the like (see, e.g., US Patent Publ. No. US 2020/0353056 A1; McGrady, N. R. et al., Front. Pharmacol. 12:764709 (2021); Ribeiro, M. et al., Int. J. Mol. Sci 23: 2911 (2022); Ribeiro et al., Int. J. Mol. Sci. 23: 7004 (2022)); the disclosure of each of these references is incorporated herein by reference in its entirety. However, this previous work was largely silent as to the potential impact of CMP-containing formulations in treating and/or preventing other eye afflictions such as myopia, presbyopia and keratoconus, most of which require mechanical and/or surgical interventions which are often incomplete, uncomfortable or inconvenient for the patient and which often require adjustment and/or re-intervention as the patient ages.

Age-related neurodegenerative disorders in concert with acute injuries to the central nervous system (CNS) represent an increasingly debilitating burden to both those who struggle with them and the caregivers of such patients, as well as to the healthcare systems that must provide long-term care. Most pharmacological or gene-therapeutic approaches to protecting or repairing neurons and their circuits in the CNS modulate the action of one or more receptor-ligand or intracellular signaling pathways implicated in either pathogenic pathways (to slow progression) or trophic support (to counter disease progression). However, outside of the cell, there may be an equally rich but underdeveloped opportunity for new therapeutic avenues. The extracellular space represents a substantial component of all tissues and organs, including the brain. A more accurate description might be extracellular matter, for the space is filled with, among other components, an intricate and highly dynamic network of both structural and bioactive proteins that constitute the ECM, particularly collagen, which is produced in the CNS mostly by astrocytes, neurons, and vascular cells. Historically, collagen has been viewed primarily as a scaffolding protein that adds biomechanical stability. However, the ECM is also involved in maintaining cellular and tissue homeostasis and in modulating a variety of cellular activities that play critical roles in maintaining the health and normal functioning of cells, tissues, organs and organ systems.

Thus, there is a need in the art for formulations and methods of use that will overcome many of these shortcomings in traditional treatments for certain ocular diseases and disorders in humans and veterinary animals. Such formulations and methods of use would allow the use of lower doses of medication and more targeted delivery of the medications to the intended sites of action, as well as reducing the therapeutic problems or delays resulting from patient non-compliance. Finally, there is a need in the art for methods of producing such compositions that will meet the needs of the medical and patient communities in maximizing treatment efficacies while reducing costs.

### BRIEF SUMMARY OF THE INVENTION

The present inventors reasoned that since collagen disruption is associated with a variety of diseases and disorders in humans and other animals, the conjugation of a variety of therapeutic compounds and/or diagnostic compounds to collagen or collagen mimetic peptides would provide an elegant, rapid and reproducible way of overcoming many of the above-referenced limitations in treatment and diagnosis of certain physical diseases and disorders and in drug delivery. Thus, the present invention provides compositions suitable for use in methods of treatment and diagnosis of physical disorders and diseases, and provides drug delivery systems, medical devices and methods of manufacturing the same. Accordingly, the present invention meets the needs in the art as expressed hereinabove.

In one aspect, the invention provides compositions comprising one or more collagen mimetic peptides (CMPs), which in certain embodiments have been conjugated one or more therapeutic compounds and/or one or more diagnostic compounds thereby forming CMP conjugates and compositions. Such CMPs and CMP conjugates, and compositions comprising such CMPs and/or CMP conjugates, are useful in treating, preventing, ameliorating and diagnosing a variety of diseases, disorders and physical conditions in humans and veterinary animals. In certain embodiments of this aspect, the invention provides compositions comprising such CMPs and/or CMP conjugates and one or more pharmaceutically acceptable carriers, excipients or compounding agents, and optionally one or more additional therapeutic or diagnostic agents, to provide therapeutic and diagnostic compositions useful in treating, preventing, ameliorating or diagnosing certain diseases and disorders in humans and veterinary animals.

In another aspect, the invention provides methods of treating, preventing, ameliorating or diagnosing certain diseases and disorders in humans and veterinary animals, by administering the conjugates and/or compositions of the invention to a human or veterinary animal suffering from or predisposed to such diseases or disorders. Diseases and disorders suitably treated, prevented, cured, ameliorated or diagnoses according to this aspect of the invention include ocular diseases or disorders, skin diseases or disorders, cancers, gastrointestinal diseases or disorders, genitourinary tract diseases or disorders, fibrotic diseases or disorders, cardiovascular diseases or disorders, bone diseases or disorders, and rheumatic diseases or disorders.

In yet another aspect, the invention provides medical devices coated with or comprising one or more of the conjugates or compositions of the invention. In related aspects, the invention provides methods of treating, curing, preventing or ameliorating diseases or disorders in humans or veterinary animals comprising implanting one or more of the medical devices of this aspect of the invention into the human or veterinary animal, under conditions such that the disease or disorder is treated, cured, prevented or ameliorated.

In still other aspects, the invention provides methods of manufacturing the compositions, conjugates and medical devices of the invention.

Other objects, advantages, and features of the present invention will be readily apparent to those of ordinary skill in the art upon review of the description, drawings, examples and claims presented herein.

### BRIEF DESCRIPTION OF THE DRAWINGS

FIGS. 1A-1H are a series of inverted brightfield photomicrographs of dorsal root ganglia (DRG) neurons plated onto tissue culture plates that had been coated overnight with type I collagen (FIGS. 1A, 1E) or with MMP-1-digested type I collagen (FIGS. 1B-1D and 1F-1H). Following coating, plates were treated with vehicle (PBS; FIGS. 1B, 1F), or with CMP 10A (SEQ ID NO:473) (FIGS. 1C, 1G) or CMP 9C (SEQ ID NO:5) (FIGS. 1D, 1H), prior to plating of DRG explants. DRG explant cultures were incubated at 37° C. prior to photomicrography at t=24 hours (FIGS. 1A-1D) and t=48 hours (FIGS. 1E-1H) post-plating.

FIGS. 2A-2B are a pair of high-power photomicrographs of DRG explants from FIG. 1 above, measuring the explant outgrowth. FIG. 2A: DRG explant 48 hours after plating on intact collagen visualized live through multi-focal high-magnification phase contrast microscopy to optimize identification and measurement of longest neurite (arrow). FIG. 2B: Confocal micrograph of similar explant showing neurites immunolabeled for β-tubulin (red) extending from DRG neurons labeled against NeuN (green). Scale: 250 μm (FIG. 2A) or 100 μm (FIG. 2B).

FIGS. 3A-3B are a pair of bar graphs showing neurite outgrowth following CMP treatment of DRG neuronal explants. Graphs show longest neurite outgrowth (FIG. 3A) and explant growth field area (FIG. 3B) at 48 hours normalized to their values for intact collagen (n=178). Explants plated on digested collagen and treated with vehicle only (n=139) demonstrate significantly diminished neurite length (p=0.014) and field area (p<0.001) compared to intact collagen (#).

FIGS. 4A-4D are a series of confocal fluorescence photomicrographs of mouse eyes that had received intravitreous injection (FIG. 4A, 4B) or dropwise ocular surface application (FIG. 4C, 4D) of a solution of a Tide Fluor™2-conjugated (Pro-Pro-Gly)7 (SEQ ID NO:1) CMP (“TF2-CMP”). Mice were sacrificed on day 3 after surface application of TF2-CMP, or on same day as intravitreal injection, and the localization of TF2-CMP was determined. FIGS. 4A and 4C show localization of TF2-CMP extranuclearly in the ganglion cell layer of the retina (arrows: retinal blood vessels; arrowheads: nuclei of ganglion cells), while FIGS. 4B and 4D show localization of TF2-CMP in or near the inner limiting membrane (vitreous surface) of the retina.

FIGS. 5A-5B show the results of CMP-treatment of mice on nerve structure in the corneal nerve bed. FIG. 5A depicts representative pseudo colored nerve fragmentation images of the corneal nerve beds from naïve, vehicle-treated, and CMP 03A (SEQ ID NO: 1)-treated mice. Images show degree of collagen fragmentation in the sub-basal plexus (lefthand column) and epithelial terminals (central column). Dashed boxes in the photomicrographs in the central column indicate location of enlarged inset images (righthand column). FIG. 5B is a bar graph demonstrating the level of nerve growth in photomicrographs from the central subbasal layer vs. the epithelial layer.

FIGS. 6A-6C are a series of fluorescence photomicrographs demonstrating that CMPs promote axon repair following nerve crush. FIG. 6A: stitched montage of confocal micrographs of longitudinal section through the optic nerve two weeks following crush from eye receiving an intravitreal injection of vehicle (DMOS)) three days after the injury. Axons containing cholera toxin B (CTB, false color white) extend to the site of the crush (dashed line) but generally not beyond distally towards the brain (arrow). Astrocyte glia labeled for glial fibrillary acidic protein (GFAP, red) are shown for comparison. FIG. 6B: in contrast, CTB-containing axons in a section of nerve from eye receiving CMP 13A (SEQ ID NO:6) extend beyond the crush site and are apparent even at more distal locations along the nerve. Repaired axons are largely coincident with localized patches of CMP 13A (green) (FIG. 6C, arrows), visualized through its attached fluorophore. Scale=200 m (FIG. 6A) or 100 m (FIGS. 6B-6C).

FIGS. 7A-7E are a series of bar graphs demonstrating the effects of CMP treatment on axon recovery following nerve crush. Graphs show the number of CTB+ axon segments at specific distances distal to the injury site two weeks following nerve crush in individual samples (symbols in FIGS. 7A and 7B), digitally normalized to nerve width. At each location, the median number of scored axons (dashed lines) in nerves from CMP 13A (SEQ ID NO:6)-treated eyes (FIG. 7A) exceeded that in nerves from vehicle (DMSO)-treated eyes (FIG. 7B); this trend is summarized in the line graph inset in FIG. 7B. FIG. 7C depicts the number of CTB+ axon segments above the median (top 50%) from FIGS. 7A,B, averaged at distances from crush site as indicated. FIGS. 7D and 7E depict the mean length (7D) and length of longest segment (7E) of axonal segments from CMP- or vehicle-treated eyes, showing that axon segments in nerves from eyes receiving CMP treatment extended further compared to axon segments from vehicle-treated eyes, both by mean length and the longest 25 segments in each group (*, p<0.001).

FIG. 8 is a series of fluorescence photomicrographs of sections of liver from wildtype (“WT”) mice or from ApoE^{-/-} mice, examined for localization of vehicle (PBS) or fluorotagged CMP injected via tail vein. Sections were stained with DAPI (for cell nuclei visualization) and examined via fluorescence microscopy at emission wavelengths of 405 nm (for DAPI) or 488 nm (for fluoro-CMP). Row A: DAPI visualization only; Row B: DAPI+CMP visualization; Row C: CMP visualization only; Row D: higher-power magnification views of insets (white boxes) from Row C. Column 1: WT mice treated with vehicle only; Column 2: WT mice treated with 100 μM CMP; Row 3: ApoE^{-/-} mice treated with 100 μM CMP.

FIG. 9 is a higher power fluorescence micrograph of the micrograph depicted in FIG. 8, Rows C and D, Column 3. Arrows show accumulation of fluoro-tagged CMP in the basement membrane of a blood vessel in liver section from an ApoE^{-/-} mice.

FIGS. 10A-10B are a pair of fluorescence photomicrographs of sections of aortic arch from a wildtype mouse (FIG. 10A) or from an ApoE^{-/-} mouse (FIG. 10B), examined for localization of fluorotagged CMP following injection via tail vein.

FIGS. 11A-11B are a higher-power view of the aortic arch from a wildtype (FIG. 11A) or ApoE^{-/-} mouse (FIG. 11B), treated and examined for CMP localization as described for FIG. 10, also demonstrating the occlusion of the aortic arch co-localizing with CMP (arrows) indicative of disrupted collagen in the ApoE^{-/-} mouse compared to the wildtype mouse.

FIGS. 12A-12D are a series of fluorescence photomicrographs of sections of mouse retina from wildtype mice (FIGS. 12A, 12B) or ApoE^{-/-} mice treated with the antibiotic streptozotocin (STZ) (FIGS. 12C, 12D), a compound that has a preferential toxicity toward pancreatic p cells and induces diabetes and accelerates progression of atherosclerosis. Sections were examined for localization of fluorotagged CMP following injection via tail vein (green), for reactive glial astrocytes labeled via GFAP treatment, and for blood vessels labeled via isolectin treatment.

FIGS. 13A-13F are a series of fluorescence photomicrographs of sections of mouse brain from wildtype mice (FIGS. 13A-13C) or ApoE−/− mice treated with STZ (FIGS. 13D-13F) as in FIG. 12. Sections were examined for localization of fluorotagged CMP following injection via tail vein (green), for reactive glial astrocytes labeled via GFAP treatment, and for blood vessels labeled via fluorescent biotin treatment. Sections from the cerebral cortex (FIGS. 13A, 13D), hippocampus CA1 region (FIGS. 13B, 13E), and in the dentate gyrus (FIGS. 13C, 13F) were analyzed for ligand localization in both mouse phenotypes. Scale bars=100 μM

FIGS. 14A-14F are a series of fluorescence photomicrographs of sections of mouse brain from ApoE−/− mice treated with STZ and subsequently treated via tail vein injection with either PBS vehicle (FIGS. 14A-14C) or with CMP (FIGS. 14D-14F). Sections were examined for PECAM-1 (CD31) expression, an indicator of intact blood vessels, via immunolabelling. Anti-CD31 labeling of thin sections from the cerebral cortex (FIGS. 14A, 14D), hippocampus CA1 region (FIGS. 14B, 14E), and in the dentate gyrus (FIGS. 14C, 14F) was determined by fluorescence confocal microscopy. Scale bars=100 M.

FIGS. 15A-15B are a series of bar graphs showing the total CD31 staining area (FIG. 15A) and staining intensity (FIG. 15B) in the cerebral cortex, hippocampal CA1, and dentate gyrus (DG) regions of the brains of ApoE−/− mice treated with STZ and subsequently treated via tail vein injection with either PBS vehicle or with CMP. Data are presented as means SEM. *p<0.02; **p=0.001; ****p<0.0001; ns=not significant (p=0.09); two-way ANOVA.

FIGS. 16A-16F are a series of fluorescence photomicrographs of sections of mouse brain from ApoE−/− mice treated with STZ and subsequently treated via tail vein injection with either PBS vehicle (FIGS. 16A-16C) or with CMP (FIGS. 16D-16F), and then with Sulfo-NHS biotin one day prior to euthanasia to track vascular leakage. Biotin was localized in thin sections using Strepdavadin Alexa-647, and sections examined for biotin immunolabeling via fluorescence confocal microscopy. Scale bars=100 μM.

FIGS. 17A-17B are a series of bar graphs showing biotin staining intensity (FIG. 17A) and CD31 staining intensity normalized to biotin intensity (FIG. 17B) in the cerebral cortex, hippocampal CA1, and dentate gyrus (DG) regions of the brains of ApoE−/− mice treated with STZ and subsequently treated via tail vein injection with either PBS vehicle or with CMP. Data are presented as means±SEM. FIG. 17A: *p<0.02; **p=0.01; two-way ANOVA. FIG. 17B: *p=0.04; two-way ANOVA.

FIGS. 18A-18F are a series of fluorescence photomicrographs of sections of mouse brain from ApoE−/− mice treated with STZ and subsequently treated via tail vein injection with either PBS vehicle (FIGS. 18A-18C) or with CMP (FIGS. 18D-18F). Sections were examined for type IV collagen expression via immunolabelling. Anti-collagen IV labeling of thin sections from the cerebral cortex (FIGS. 18A, 18D), hippocampus CA1 region (FIGS. 18B, 18E), and in the dentate gyrus (FIGS. 18C, 18F) was determined by fluorescence confocal microscopy. Scale bars=30 μM.

FIG. 19 is a series of bar graphs showing type IV collagen immunofluorescence staining intensity of sections such as those shown in FIG. 18, in the cerebral cortex, hippocampal CA1, and dentate gyrus (DG) regions of the brains of ApoE−/− mice treated with STZ and subsequently treated via tail vein injection with either PBS vehicle or with CMP. Data are presented as means±SEM. *p=0.04; **p=0.01; Mann-Whitney non-parametric t-test.

FIGS. 20A-20H are a series of fluorescence photomicrographs of sections of mouse brain from ApoE−/− mice treated with STZ and subsequently treated via tail vein injection with either PBS vehicle (FIGS. 20A-20D) or with CMP (FIGS. 20E-20H). Sections from the hippocampal CA1 region (FIGS. 20A, 20B, 20E and 20F) and dentate gyrus (DG) region (FIGS. 20C, 20D, 20G and 20H) were examined for astroglia using anti-GFAP immunolabeling (all panels) and for vascular integrity via biotin immunolabeling (FIGS. 20B, 20D, 20F, 20H). Labeling was determined by fluorescence confocal microscopy. Scale bars=30 M.

FIGS. 21A-21F are a series of bar graphs showing the number of astrocytes present, and interactions between astrocytes and blood vessels, in sections of the hippocampal CA1 (FIGS. 21A-21C) and dentate gyrus (DG) (FIGS. 21D-21F) regions of the brains of ApoE−/− mice treated with STZ and subsequently treated via tail vein injection with either PBS vehicle or with CMP, such as those shown in FIG. 20. FIGS. 21A and 21D: GFAP staining intensity, a measure of astrocyte presence. FIGS. 21B and 21E: quantitation of the number of astrocytes per image (*p=0.02). FIGS. 21C and 21F: measurement of the shortest distance from the centroid of a given astrocyte to the nearest blood vessel per image. ns=nonsignificant, p=0.07; *p=0.02; ***p=0.0004; ****p<0.0001. Data are presented as means±SEM; Mann-Whitney non-parametric t-test.

### DETAILED DESCRIPTION OF THE INVENTION

Unless defined otherwise, all technical and scientific terms used herein have the same meanings as commonly understood by one of ordinary skill in the arts to which this invention belongs. Although any methods and materials similar or equivalent to those described herein can be used in the practice or testing of the present invention, the preferred methods and materials are described hereinafter.

According to a first aspect, the invention provides compositions suitable for use in a medicament for treating or preventing a disease, disorder, structural abnormality or injury in a human or veterinary animal in need of treatment or prevention of such as a disease, disorder, structural abnormality or injury. In certain embodiments, the compositions provided by the invention comprise (a) at least one collagen mimetic peptide (CMP) attached to at least one additional therapeutic compound (TC) to form a CMP-TC conjugate, and (b) one or more pharmaceutically suitable carriers. In related aspects, the invention provides compositions suitable for use in a diagnostic agent suitable for diagnosing or detecting a disease, disorder, structural abnormality or injury in a human or veterinary animal in need thereof. In certain embodiments, the compositions provided by the invention comprise (a) at least one collagen mimetic peptide (CMP) attached to at least one diagnostic compound or agent (DC) to form a CMP-DC conjugate, and (b) one or more pharmaceutically suitable carriers. In other related embodiments, the compositions provided by the invention comprise (a) at least one collagen mimetic peptide (CMP) and (b) at least one additional therapeutic compound, wherein the CMP and the at least one additional therapeutic compound are admixed in a formulation, or “co-formulated,” optionally together with one or more pharmaceutically suitable carriers. In analogous embodiments, the compositions provided by the invention comprise (a) at least one collagen mimetic peptide (CMP) and (b) at least one diagnostic compound or agent, such as a labeling compound or agent, wherein the CMP and the at least one diagnostic compound or agent are admixed in a formulation, or “co-formulated,” optionally together with one or more pharmaceutically suitable carriers, for use in one or more diagnostic methods of the invention.

In certain embodiments of the invention, the collagen mimetic peptide comprises, consists essentially of or consists of an amino acid sequence that is a multimeric repeat of a specific tripeptide having a sequence (Xaa-Yaa-Gly)ₙ (SEQ ID NO: 417), wherein Xaa is independently selected from the group consisting of proline, 4S-hydroxyproline, fluoroproline, chloroproline, lysine, cysteine and methionine; wherein Yaa is independently selected from the group consisting of proline, 4R-hydroxyproline, fluoroproline, chloroproline, lysine, cysteine and methionine; wherein Gly is a glycine residue; and wherein n is an integer ranging from 1 to 20, such as from 3 to 15, from 5 to 15, or from 5 to 10, and is preferably 5, 6, 7, 8, 9 or 10.

In other embodiments, the invention provides collagen mimetic peptides that comprise one or more alternative amino acids in place of at least one of the amino acids set forth in the tripeptide shown in SEQ ID NO:417, including but not limited to alanine (Ala), glutamine (Gln), glutamic acid (Glu), asparagine (Asn) and aspartic acid (Asp).

In certain embodiments of the invention, the collagen mimetic peptide comprises, consists essentially of or consists of an amino acid sequence that is or corresponds to a 21-mer comprising seven repeats of a three amino acid sequence of proline-proline-glycine ((Pro-Pro-Gly)₇), i.e., an amino acid sequence of: Pro-Pro-Gly-Pro-Pro-Gly-Pro-Pro-Gly-Pro-Pro-Gly-Pro-Pro-Gly-Pro-Pro-Gly-Pro-Pro-Gly (SEQ ID NO:1).

In certain other embodiments of the invention, the collagen mimetic peptide comprises, consists essentially of or consists of an amino acid sequence that is or corresponds to a 21-mer comprising seven repeats of a three amino acid sequence in which hydroxyproline (Hyp), and preferably a 4S-hydroxyproline residue, has been substituted for proline₁ in SEQ ID NO:1, yielding a sequence of seven repeats of 4S-hydroxyproline-proline-glycine ((Hyp-Pro-Gly)₇), i.e., an amino acid sequence of: Hyp-Pro-Gly-Hyp-Pro-Gly-Hyp-Pro-Gly-Hyp-Pro-Gly-Hyp-Pro-Gly-Hyp-Pro-Gly-Hyp-Pro-Gly (SEQ ID NO:2).

In certain other embodiments of the invention, the collagen mimetic peptide comprises, consists essentially of or consists of an amino acid sequence that is or corresponds to a 21-mer comprising seven repeats of a three amino acid sequence in which Hyp, and preferably a 4S-hydroxyproline residue, has been substituted for proline₂ in SEQ ID NO:1, yielding a sequence of seven repeats of 4S-hydroxyproline-proline-glycine ((Pro-Hyp-Gly)₇), i.e., an amino acid sequence of: Pro-Hyp-Gly-Pro-Hyp-Gly-Pro-Hyp-Gly-Pro-Hyp-Gly-Pro-Hyp-Gly-Pro-Hyp-Gly-Pro-Hyp-Gly (SEQ ID NO:3).

In certain other embodiments of the invention, the collagen mimetic peptide comprises, consists essentially of or consists of an amino acid sequence that is or corresponds to a 21-mer comprising seven repeats of a three amino acid sequence in which fluoroproline (Flp) has been substituted for proline₁ in SEQ ID NO:1, yielding a sequence of seven repeats of fluoroproline-proline-glycine ((Flp-Pro-Gly)₇), i.e., an amino acid sequence of: Flp-Pro-Gly-Flp-Pro-Gly-Flp-Pro-Gly-Flp-Pro-Gly-Flp-Pro-Gly-Flp-Pro-Gly-Flp-Pro-Gly (SEQ ID NO:4).

In certain other embodiments of the invention, the collagen mimetic peptide comprises, consists essentially of or consists of an amino acid sequence that is or corresponds to a 21-mer comprising seven repeats of a three amino acid sequence in which Flp has been substituted for proline₂ in SEQ ID NO:1, yielding a sequence of seven repeats of proline-fluoroproline-glycine ((Pro-Flp-Gly)₇), i.e., an amino acid sequence of: Pro-Flp-Gly-Pro-Flp-Gly-Pro-Flp-Gly-Pro-Flp-Gly-Pro-Flp-Gly-Pro-Flp-Gly-Pro-Flp-Gly (SEQ ID NO:5).

In certain other embodiments of the invention, the collagen mimetic peptide comprises, consists essentially of or consists of an amino acid sequence that is or corresponds to a 21-mer comprising seven repeats of a three amino acid sequence in which fluoroproline (Flp) has been substituted for proline₁ in SEQ ID NO:1 and Hyp has been substituted for proline₂ in SEQ ID NO:1, yielding a sequence of seven repeats of fluoroproline-hydroxyproline-glycine ((Flp-Hyp-Gly)₇), i.e., an amino acid sequence of: Flp-Hyp-Gly-Flp-Hyp-Gly-Flp-Hyp-Gly-Flp-Hyp-Gly-Flp-Hyp-Gly-Flp-Hyp-Gly-Flp-Hyp-Gly (SEQ ID NO:6).

In CMPs containing Flp, the Flp moiety may be in the 4-cis or 4-trans configuration, and preferably is in the 4-cis configuration.

In certain other embodiments of the invention, the collagen mimetic peptide may comprise, consist of or have an amino acid sequence that is or corresponds to a 21-mer comprising seven repeats of a three amino acid sequence in which chloroproline (Clp) has been substituted for proline₁ in SEQ ID NO:1, yielding a sequence of seven repeats of chloroproline-proline-glycine ((Clp-Pro-Gly)₇), i.e., an amino acid sequence of: Clp-Pro-Gly-Clp-Pro-Gly-Clp-Pro-Gly-Clp-Pro-Gly-Clp-Pro-Gly-Clp-Pro-Gly-Clp-Pro-Gly (SEQ ID NO:7).

In certain other embodiments of the invention, the collagen mimetic peptide may comprise, consist of or have an amino acid sequence that is or corresponds to a 21-mer comprising seven repeats of a three amino acid sequence in which chloroproline (Clp) has been substituted for proline₂ in SEQ ID NO:1, yielding a sequence of seven repeats of proline-chloroproline-glycine ((Pro-Clp-Gly)₇), i.e., an amino acid sequence of: Pro-Clp-Gly-Pro-Clp-Gly-Pro-Clp-Gly-Pro-Clp-Gly-Pro-Clp-Gly-Pro-Clp-Gly-Pro-Clp-Gly (SEQ ID NO:8).

In certain other embodiments of the invention, the collagen mimetic peptide comprises, consists essentially of or consists of an amino acid sequence that is or corresponds to a 21-mer comprising seven repeats of a three amino acid sequence in which Clp has been substituted for proline₁ in SEQ ID NO:1 and Hyp has been substituted for proline₂ in SEQ ID NO:1, yielding a sequence of seven repeats of chloroproline-hydroxyproline-glycine ((Clp-Hyp-Gly)₇), i.e., an amino acid sequence of: Clp-Hyp-Gly-Clp-Hyp-Gly-Clp-Hyp-Gly-Clp-Hyp-Gly-Clp-Hyp-Gly-Clp-Hyp-Gly-Clp-Hyp-Gly (SEQ ID NO:9).

In CMPs containing Clp, the Clp moiety may be in the 4-cis or 4-trans configuration, and preferably is in the 4-cis configuration.

In certain other embodiments of the invention, the collagen mimetic peptide may comprise, consist of or have an amino acid sequence that is or corresponds to a 21-mer of any one of SEQ ID NOs:1-9, in which at least one cysteine (Cys) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 10)
Pro-Pro-Gly-Pro-Pro-Gly-Cys-Pro-Gly-Pro-Pro-Gly-
Pro-Pro-Gly-Pro-Pro-Gly-Pro-Pro-Gly;
(SEQ ID NO: 11)
Hyp-Pro-Gly-Hyp-Pro-Gly-Cys-Pro-Gly-Hyp-Pro-Gly-
Hyp-Pro-Gly-Hyp-Pro-Gly-Hyp-Pro-Gly;
(SEQ ID NO: 12)
Pro-Hyp-Gly-Pro-Hyp-Gly-Pro-Cys-Gly-Pro-Hyp-Gly-
Pro-Hyp-Gly-Pro-Hyp-Gly-Pro-Hyp-Gly;
(SEQ ID NO: 13)
Pro-Flp-Gly-Pro-Flp-Gly-Pro-Cys-Gly-Pro-Flp-Gly-
Pro-Flp-Gly-Pro-Flp-Gly-Pro-Flp-Gly;
(SEQ ID NO: 14)
Pro-Clp-Gly-Pro-Clp-Gly-Pro-Cys-Gly-Pro-Clp-Gly-
Pro-Clp-Gly-Pro-Clp-Gly-Pro-Clp-Gly;
(SEQ ID NO: 15)
Cys-Pro-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 16)
Pro-Cys-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 17)
Pro-Pro-Gly-Cys-Pro-Gly-Pro-Pro-Gly-Pro-Pro-Gly-
Pro-Pro-Gly-Pro-Pro-Gly-Pro-Pro-Gly;
(SEQ ID NO: 18)
Pro-Pro-Gly-Pro-Cys-Gly-Pro-Pro-Gly-Pro-Pro-Gly-
Pro-Pro-Gly-Pro-Pro-Gly-Pro-Pro-Gly;
(SEQ ID NO: 19)
Pro-Pro-Gly-Pro-Pro-Gly-Pro-Cys-Gly-Pro-Pro-Gly-
Pro-Pro-Gly-Pro-Pro-Gly-Pro-Pro-Gly;
(SEQ ID NO: 20)
Pro-Pro-Gly-Pro-Pro-Gly-Pro-Pro-Gly-Cys-Pro-Gly-
Pro-Pro-Gly-Pro-Pro-Gly-Pro-Pro-Gly;
(SEQ ID NO: 21)
Pro-Pro-Gly-Pro-Pro-Gly-Pro-Pro-Gly-Pro-Cys-Gly-
Pro-Pro-Gly-Pro-Pro-Gly-Pro-Pro-Gly;
(SEQ ID NO: 22)
Pro-Pro-Gly-Pro-Pro-Gly-Pro-Pro-Gly-Pro-Pro-Gly-
Cys-Pro-Gly-Pro-Pro-Gly-Pro-Pro-Gly;
(SEQ ID NO: 23)
Pro-Pro-Gly-Pro-Pro-Gly-Pro-Pro-Gly-Pro-Pro-Gly-
Pro-Cys-Gly-Pro-Pro-Gly-Pro-Pro-Gly;
(SEQ ID NO: 24)
Pro-Pro-Gly-Pro-Pro-Gly-Pro-Pro-Gly-Pro-Pro-Gly-
Pro-Pro-Gly-Cys-Pro-Gly-Pro-Pro-Gly;
(SEQ ID NO: 25)
Pro-Pro-Gly-Pro-Pro-Gly-Pro-Pro-Gly-Pro-Pro-Gly-
Pro-Pro-Gly-Pro-Cys-Gly-Cys-Pro-Gly;
(SEQ ID NO: 26)
Pro-Pro-Gly-Pro-Pro-Gly-Pro-Pro-Gly-Pro-Pro-Gly-
Pro-Pro-Gly-Pro-Pro-Gly-Cys-Pro-Gly;
(SEQ ID NO: 27)
Pro-Pro-Gly-Pro-Pro-Gly-Pro-Pro-Gly-Pro-Pro-Gly-
Pro-Pro-Gly-Pro-Pro-Gly-Pro-Cys-Gly;
(SEQ ID NO: 28)
Cys-Pro-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 29)
Hyp-Cys-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 30)
Hyp-Pro-Gly-Cys-Pro-Gly-Hyp-Pro-Gly-Hyp-Pro-Gly-
Hyp-Pro-Gly-Hyp-Pro-Gly-Hyp-Pro-Gly;
(SEQ ID NO: 31)
Hyp-Pro-Gly-Hyp-Cys-Gly-Hyp-Pro-Gly-Hyp-Pro-Gly-
Hyp-Pro-Gly-Hyp-Pro-Gly-Hyp-Pro-Gly;
(SEQ ID NO: 32)
Hyp-Pro-Gly-Hyp-Pro-Gly-Hyp-Cys-Gly-Hyp-Pro-Gly-
Hyp-Pro-Gly-Hyp-Pro-Gly-Hyp-Pro-Gly;
(SEQ ID NO: 33)
Hyp-Pro-Gly-Hyp-Pro-Gly-Hyp-Pro-Gly-Cys-Pro-Gly-
Hyp-Pro-Gly-Hyp-Pro-Gly-Hyp-Pro-Gly;
(SEQ ID NO: 34)
Hyp-Pro-Gly-Hyp-Pro-Gly-Hyp-Pro-Gly-Hyp-Cys-Gly-
Hyp-Pro-Gly-Hyp-Pro-Gly-Hyp-Pro-Gly;
(SEQ ID NO: 35)
Hyp-Pro-Gly-Hyp-Pro-Gly-Hyp-Pro-Gly-Hyp-Pro-Gly-
Cys-Pro-Gly-Hyp-Pro-Gly-Hyp-Pro-Gly;
(SEQ ID NO: 36)
Hyp-Pro-Gly-Hyp-Pro-Gly-Hyp-Pro-Gly-Hyp-Pro-Gly-
Hyp-Cys-Gly-Hyp-Pro-Gly-Hyp-Pro-Gly;
(SEQ ID NO: 37)
Hyp-Pro-Gly-Hyp-Pro-Gly-Hyp-Pro-Gly-Hyp-Pro-Gly-
Hyp-Pro-Gly-Cys-Pro-Gly-Hyp-Pro-Gly;
(SEQ ID NO: 38)
Hyp-Pro-Gly-Hyp-Pro-Gly-Hyp-Pro-Gly-Hyp-Pro-Gly-
Hyp-Pro-Gly-Hyp-Cys-Gly-Hyp-Pro-Gly;
(SEQ ID NO: 39)
Hyp-Pro-Gly-Hyp-Pro-Gly-Hyp-Pro-Gly-Hyp-Pro-Gly-
Hyp-Pro-Gly-Hyp-Pro-Gly-Cys-Pro-Gly;
(SEQ ID NO: 40)
Hyp-Pro-Gly-Hyp-Pro-Gly-Hyp-Pro-Gly-Hyp-Pro-Gly-
Hyp-Pro-Gly-Hyp-Pro-Gly-Hyp-Cys-Gly;
(SEQ ID NO: 41)
Cys-Hyp-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 42)
Pro-Cys-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 43)
Pro-Hyp-Gly-Cys-Hyp-Gly-Pro-Hyp-Gly-Pro-Hyp-Gly-
Pro-Hyp-Gly-Pro-Hyp-Gly-Pro-Hyp-Gly;
(SEQ ID NO: 44)
Pro-Hyp-Gly-Pro-Cys-Gly-Pro-Hyp-Gly-Pro-Hyp-Gly-
Pro-Hyp-Gly-Pro-Hyp-Gly-Pro-Hyp-Gly;
(SEQ ID NO: 45)
Pro-Hyp-Gly-Pro-Hyp-Gly-Cys-Hyp-Gly-Pro-Hyp-Gly-
Pro-Hyp-Gly-Pro-Hyp-Gly-Pro-Hyp-Gly;
(SEQ ID NO: 46)
Pro-Hyp-Gly-Pro-Hyp-Gly-Pro-Hyp-Gly-Cys-Hyp-Gly-
Pro-Hyp-Gly-Pro-Hyp-Gly-Pro-Hyp-Gly;
(SEQ ID NO: 47)
Pro-Hyp-Gly-Pro-Hyp-Gly-Pro-Hyp-Gly-Pro-Cys-Gly-
Pro-Hyp-Gly-Pro-Hyp-Gly-Pro-Hyp-Gly;
(SEQ ID NO: 48)
Pro-Hyp-Gly-Pro-Hyp-Gly-Pro-Hyp-Gly-Pro-Hyp-Gly-
Cys-Hyp-Gly-Pro-Hyp-Gly-Pro-Hyp-Gly;
(SEQ ID NO: 49)
Pro-Hyp-Gly-Pro-Hyp-Gly-Pro-Hyp-Gly-Pro-Hyp-Gly-
Pro-Cys-Gly-Pro-Hyp-Gly-Pro-Hyp-Gly;
(SEQ ID NO: 50)
Pro-Hyp-Gly-Pro-Hyp-Gly-Pro-Hyp-Gly-Pro-Hyp-Gly-
Pro-Hyp-Gly-Cys-Hyp-Gly-Pro-Hyp-Gly;
(SEQ ID NO: 51)
Pro-Hyp-Gly-Pro-Hyp-Gly-Pro-Hyp-Gly-Pro-Hyp-Gly-
Pro-Hyp-Gly-Pro-Cys-Gly-Pro-Hyp-Gly;
(SEQ ID NO: 52)
Pro-Hyp-Gly-Pro-Hyp-Gly-Pro-Hyp-Gly-Pro-Hyp-Gly-
Pro-Hyp-Gly-Pro-Hyp-Gly-Cys-Hyp-Gly;
(SEQ ID NO: 53)
Pro-Hyp-Gly-Pro-Hyp-Gly-Pro-Hyp-Gly-Pro-Hyp-Gly-
Pro-Hyp-Gly-Pro-Hyp-Gly-Pro-Cys-Gly;
(SEQ ID NO: 54)
Cys-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 55)
Pro-Cys-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 56)
Pro-Flp-Gly-Cys-Flp-Gly-Pro-Flp-Gly-Pro-Flp-Gly-
Pro-Flp-Gly-Pro-Flp-Gly-Pro-Flp-Gly;
(SEQ ID NO: 57)
Pro-Flp-Gly-Pro-Cys-Gly-Pro-Flp-Gly-Pro-Flp-Gly-
Pro-Flp-Gly-Pro-Flp-Gly-Pro-Flp-Gly;
(SEQ ID NO: 58)
Pro-Flp-Gly-Pro-Flp-Gly-Cys-Flp-Gly-Pro-Flp-Gly-
Pro-Flp-Gly-Pro-Flp-Gly-Pro-Flp-Gly;
(SEQ ID NO: 59)
Pro-Flp-Gly-Pro-Flp-Gly-Pro-Flp-Gly-Cys-Flp-Gly-
Pro-Flp-Gly-Pro-Flp-Gly-Pro-Flp-Gly;
(SEQ ID NO: 60)
Pro-Flp-Gly-Pro-Flp-Gly-Pro-Flp-Gly-Pro-Cys-Gly-
Pro-Flp-Gly-Pro-Flp-Gly-Pro-Flp-Gly;
(SEQ ID NO: 61)
Pro-Flp-Gly-Pro-Flp-Gly-Pro-Flp-Gly-Pro-Flp-Gly-
Cys-Flp-Gly-Pro-Flp-Gly-Pro-Flp-Gly;
(SEQ ID NO: 62)
Pro-Flp-Gly-Pro-Flp-Gly-Pro-Flp-Gly-Pro-Flp-Gly-
Pro-Cys-Gly-Pro-Flp-Gly-Pro-Flp-Gly;
(SEQ ID NO: 63)
Pro-Flp-Gly-Pro-Flp-Gly-Pro-Flp-Gly-Pro-Flp-Gly-
Pro-Flp-Gly-Cys-Flp-Gly-Pro-Flp-Gly;
(SEQ ID NO: 64)
Pro-Flp-Gly-Pro-Flp-Gly-Pro-Flp-Gly-Pro-Flp-Gly-
Pro-Flp-Gly-Pro-Cys-Gly-Pro-Flp-Gly;
(SEQ ID NO: 65)
Pro-Flp-Gly-Pro-Flp-Gly-Pro-Flp-Gly-Pro-Flp-Gly-
Pro-Flp-Gly-Pro-Flp-Gly-Cys-Flp-Gly;
(SEQ ID NO: 66)
Pro-Flp-Gly-Pro-Flp-Gly-Pro-Flp-Gly-Pro-Flp-Gly-
Pro-Flp-Gly-Pro-Flp-Gly-Pro-Cys-Gly;
(SEQ ID NO: 67)
Cys-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 68)
Pro-Cys-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 69)
Pro-Flp-Gly-Cys-Flp-Gly-Pro-Flp-Gly-Pro-Flp-Gly-
Pro-Flp-Gly-Pro-Flp-Gly-Pro-Flp-Gly;
(SEQ ID NO: 70)
Pro-Flp-Gly-Pro-Cys-Gly-Pro-Flp-Gly-Pro-Flp-Gly-
Pro-Flp-Gly-Pro-Flp-Gly-Pro-Flp-Gly;
(SEQ ID NO: 71)
Pro-Flp-Gly-Pro-Flp-Gly-Cys-Flp-Gly-Pro-Flp-Gly-
Pro-Flp-Gly-Pro-Flp-Gly-Pro-Flp-Gly;
(SEQ ID NO: 72)
Pro-Flp-Gly-Pro-Flp-Gly-Pro-Cys-Gly-Pro-Flp-Gly-
Pro-Flp-Gly-Pro-Flp-Gly-Pro-Flp-Gly;
(SEQ ID NO: 73)
Pro-Flp-Gly-Pro-Flp-Gly-Pro-Flp-Gly-Cys-Flp-Gly-
Pro-Flp-Gly-Pro-Flp-Gly-Pro-Flp-Gly;
(SEQ ID NO: 74)
Pro-Flp-Gly-Pro-Flp-Gly-Pro-Flp-Gly-Pro-Cys-Gly-
Pro-Flp-Gly-Pro-Flp-Gly-Pro-Flp-Gly;
(SEQ ID NO: 75)
Pro-Flp-Gly-Pro-Flp-Gly-Pro-Flp-Gly-Pro-Flp-Gly-
Cys-Flp-Gly-Pro-Flp-Gly-Pro-Flp-Gly;
(SEQ ID NO: 76)
Pro-Flp-Gly-Pro-Flp-Gly-Pro-Flp-Gly-Pro-Flp-Gly-
Pro-Cys-Gly-Pro-Flp-Gly-Pro-Flp-Gly;
(SEQ ID NO: 77)
Pro-Flp-Gly-Pro-Flp-Gly-Pro-Flp-Gly-Pro-Flp-Gly-
Pro-Flp-Gly-Cys-Flp-Gly-Pro-Flp-Gly;
(SEQ ID NO: 78)
Pro-Flp-Gly-Pro-Flp-Gly-Pro-Flp-Gly-Pro-Flp-Gly-
Pro-Flp-Gly-Pro-Cys-Gly-Pro-Flp-Gly;
(SEQ ID NO: 79)
Pro-Flp-Gly-Pro-Flp-Gly-Pro-Flp-Gly-Pro-Flp-Gly-
Pro-Flp-Gly-Pro-Flp-Gly-Cys-Flp-Gly;
(SEQ ID NO: 80)
Pro-Flp-Gly-Pro-Flp-Gly-Pro-Flp-Gly-Pro-Flp-Gly-
Pro-Flp-Gly-Pro-Flp-Gly-Pro-Cys-Gly;
(SEQ ID NO: 81)
Cys-Hyp-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 82)
Flp-Cys-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 83)
Flp-Hyp-Gly-Cys-Hyp-Gly-Flp-Hyp-Gly-Flp-Hyp-Gly-
Flp-Hyp-Gly-Flp-Hyp-Gly-Flp-Hyp-Gly;
(SEQ ID NO: 84)
Flp-Hyp-Gly-Flp-Cys-Gly-Flp-Hyp-Gly-Flp-Hyp-Gly-
Flp-Hyp-Gly-Flp-Hyp-Gly-Flp-Hyp-Gly;
(SEQ ID NO: 85)
Flp-Hyp-Gly-Flp-Hyp-Gly-Cys-Hyp-Gly-Flp-Hyp-Gly-
Flp-Hyp-Gly-Flp-Hyp-Gly-Flp-Hyp-Gly;
(SEQ ID NO: 86)
Flp-Hyp-Gly-Flp-Hyp-Gly-Flp-Cys-Gly-Flp-Hyp-Gly-
Flp-Hyp-Gly-Flp-Hyp-Gly-Flp-Hyp-Gly;
(SEQ ID NO: 87)
Flp-Hyp-Gly-Flp-Hyp-Gly-Flp-Hyp-Gly-Cys-Hyp-Gly-
Flp-Hyp-Gly-Flp-Hyp-Gly-Flp-Hyp-Gly;
(SEQ ID NO: 88)
Flp-Hyp-Gly-Flp-Hyp-Gly-Flp-Hyp-Gly-Flp-Cys-Gly-
Flp-Hyp-Gly-Flp-Hyp-Gly-Flp-Hyp-Gly;
(SEQ ID NO: 89)
Flp-Hyp-Gly-Flp-Hyp-Gly-Flp-Hyp-Gly-Flp-Hyp-Gly-
Cys-Hyp-Gly-Flp-Hyp-Gly-Flp-Hyp-Gly;
(SEQ ID NO: 90)
Flp-Hyp-Gly-Flp-Hyp-Gly-Flp-Hyp-Gly-Flp-Hyp-Gly-
Flp-Cys-Gly-Flp-Hyp-Gly-Flp-Hyp-Gly;
(SEQ ID NO: 91)
Flp-Hyp-Gly-Flp-Hyp-Gly-Flp-Hyp-Gly-Flp-Hyp-Gly-
Flp-Hyp-Gly-Cys-Hyp-Gly-Flp-Hyp-Gly;
(SEQ ID NO: 92)
Flp-Hyp-Gly-Flp-Hyp-Gly-Flp-Hyp-Gly-Flp-Hyp-Gly-
Flp-Hyp-Gly-Flp-Cys-Gly-Flp-Hyp-Gly;
(SEQ ID NO: 93)
Flp-Hyp-Gly-Flp-Hyp-Gly-Flp-Hyp-Gly-Flp-Hyp-Gly-
Flp-Hyp-Gly-Flp-Hyp-Gly-Cys-Hyp-Gly;
(SEQ ID NO: 94)
Flp-Hyp-Gly-Flp-Hyp-Gly-Flp-Hyp-Gly-Flp-Hyp-Gly-
Flp-Hyp-Gly-Flp-Hyp-Gly-Flp-Cys-Gly;
(SEQ ID NO: 95)
Cys-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 96)
Pro-Cys-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 97)
Pro-Clp-Gly-Cys-Clp-Gly-Pro-Clp-Gly-Pro-Clp-Gly-
Pro-Clp-Gly-Pro-Clp-Gly-Pro-Clp-Gly;
(SEQ ID NO: 98)
Pro-Clp-Gly-Pro-Cys-Gly-Pro-Clp-Gly-Pro-Clp-Gly-
Pro-Clp-Gly-Pro-Clp-Gly-Pro-Clp-Gly;
(SEQ ID NO: 99)
Pro-Clp-Gly-Pro-Clp-Gly-Cys-Clp-Gly-Pro-Clp-Gly-
Pro-Clp-Gly-Pro-Clp-Gly-Pro-Clp-Gly;
(SEQ ID NO: 100)
Pro-Clp-Gly-Pro-Clp-Gly-Pro-Clp-Gly-Cys-Clp-Gly-
Pro-Clp-Gly-Pro-Clp-Gly-Pro-Clp-Gly;
(SEQ ID NO: 101)
Pro-Clp-Gly-Pro-Clp-Gly-Pro-Clp-Gly-Pro-Cys-Gly-
Pro-Clp-Gly-Pro-Clp-Gly-Pro-Clp-Gly;
(SEQ ID NO: 102)
Pro-Clp-Gly-Pro-Clp-Gly-Pro-Clp-Gly-Pro-Clp-Gly-
Cys-Clp-Gly-Pro-Clp-Gly-Pro-Clp-Gly;
(SEQ ID NO: 103)
Pro-Clp-Gly-Pro-Clp-Gly-Pro-Clp-Gly-Pro-Clp-Gly-
Pro-Cys-Gly-Pro-Clp-Gly-Pro-Clp-Gly;
(SEQ ID NO: 104)
Pro-Clp-Gly-Pro-Clp-Gly-Pro-Clp-Gly-Pro-Clp-Gly-
Pro-Clp-Gly-Cys-Clp-Gly-Pro-Clp-Gly;
(SEQ ID NO: 105)
Pro-Clp-Gly-Pro-Clp-Gly-Pro-Clp-Gly-Pro-Clp-Gly-
Pro-Clp-Gly-Pro-Cys-Gly-Pro-Clp-Gly;
(SEQ ID NO: 106)
Pro-Clp-Gly-Pro-Clp-Gly-Pro-Clp-Gly-Pro-Clp-Gly-
Pro-Clp-Gly-Pro-Clp-Gly-Cys-Clp-Gly;
(SEQ ID NO: 107)
Pro-Clp-Gly-Pro-Clp-Gly-Pro-Clp-Gly-Pro-Clp-Gly-
Pro-Clp-Gly-Pro-Clp-Gly-Pro-Cys-Gly;
(SEQ ID NO: 108)
Cys-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 109)
Pro-Cys-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 110)
Pro-Clp-Gly-Cys-Clp-Gly-Pro-Clp-Gly-Pro-Clp-Gly-
Pro-Clp-Gly-Pro-Clp-Gly-Pro-Clp-Gly;
(SEQ ID NO: 111)
Pro-Clp-Gly-Pro-Cys-Gly-Pro-Clp-Gly-Pro-Clp-Gly-
Pro-Clp-Gly-Pro-Clp-Gly-Pro-Clp-Gly;
(SEQ ID NO: 112)
Pro-Clp-Gly-Pro-Clp-Gly-Cys-Clp-Gly-Pro-Clp-Gly-
Pro-Clp-Gly-Pro-Clp-Gly-Pro-Clp-Gly;
(SEQ ID NO: 113)
Pro-Clp-Gly-Pro-Clp-Gly-Pro-Cys-Gly-Pro-Clp-Gly-
Pro-Clp-Gly-Pro-Clp-Gly-Pro-Clp-Gly;
(SEQ ID NO: 114)
Pro-Clp-Gly-Pro-Clp-Gly-Pro-Clp-Gly-Cys-Clp-Gly-
Pro-Clp-Gly-Pro-Clp-Gly-Pro-Clp-Gly;
(SEQ ID NO: 115)
Pro-Clp-Gly-Pro-Clp-Gly-Pro-Clp-Gly-Pro-Cys-Gly-
Pro-Clp-Gly-Pro-Clp-Gly-Pro-Clp-Gly;
(SEQ ID NO: 116)
Pro-Clp-Gly-Pro-Clp-Gly-Pro-Clp-Gly-Pro-Clp-Gly-
Cys-Clp-Gly-Pro-Clp-Gly-Pro-Clp-Gly;
(SEQ ID NO: 117)
Pro-Clp-Gly-Pro-Clp-Gly-Pro-Clp-Gly-Pro-Clp-Gly-
Pro-Cys-Gly-Pro-Clp-Gly-Pro-Clp-Gly;
(SEQ ID NO: 118)
Pro-Clp-Gly-Pro-Clp-Gly-Pro-Clp-Gly-Pro-Clp-Gly-
Pro-Clp-Gly-Cys-Clp-Gly-Pro-Clp-Gly;
(SEQ ID NO: 119)
Pro-Clp-Gly-Pro-Clp-Gly-Pro-Clp-Gly-Pro-Clp-Gly-
Pro-Clp-Gly-Pro-Cys-Gly-Pro-Clp-Gly;
(SEQ ID NO: 120)
Pro-Clp-Gly-Pro-Clp-Gly-Pro-Clp-Gly-Pro-Clp-Gly-
Pro-Clp-Gly-Pro-Clp-Gly-Cys-Clp-Gly;
(SEQ ID NO: 121)
Pro-Clp-Gly-Pro-Clp-Gly-Pro-Clp-Gly-Pro-Clp-Gly-
Pro-Clp-Gly-Pro-Clp-Gly-Pro-Cys-Gly;
(SEQ ID NO: 122)
Cys-Hyp-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 123)
Clp-Cys-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 124)
Clp-Hyp-Gly-Cys-Hyp-Gly-Clp-Hyp-Gly-Clp-Hyp-Gly-
Clp-Hyp-Gly-Clp-Hyp-Gly-Clp-Hyp-Gly;
(SEQ ID NO: 125)
Clp-Hyp-Gly-Clp-Cys-Gly-Clp-Hyp-Gly-Clp-Hyp-Gly-
Clp-Hyp-Gly-Clp-Hyp-Gly-Clp-Hyp-Gly;
(SEQ ID NO: 126)
Clp-Hyp-Gly-Clp-Hyp-Gly-Cys-Hyp-Gly-Clp-Hyp-Gly-
Clp-Hyp-Gly-Clp-Hyp-Gly-Clp-Hyp-Gly;
(SEQ ID NO: 127)
Clp-Hyp-Gly-Clp-Hyp-Gly-Clp-Cys-Gly-Clp-Hyp-Gly-
Clp-Hyp-Gly-Clp-Hyp-Gly-Clp-Hyp-Gly;
(SEQ ID NO: 128)
Clp-Hyp-Gly-Clp-Hyp-Gly-Clp-Hyp-Gly-Cys-Hyp-Gly-
Clp-Hyp-Gly-Clp-Hyp-Gly-Clp-Hyp-Gly;
(SEQ ID NO: 129)
Clp-Hyp-Gly-Clp-Hyp-Gly-Clp-Hyp-Gly-Clp-Cys-Gly-
Clp-Hyp-Gly-Clp-Hyp-Gly-Clp-Hyp-Gly;
(SEQ ID NO: 130)
Clp-Hyp-Gly-Clp-Hyp-Gly-Clp-Hyp-Gly-Clp-Hyp-Gly-
Cys-Hyp-Gly-Clp-Hyp-Gly-Clp-Hyp-Gly;
(SEQ ID NO: 131)
Clp-Hyp-Gly-Clp-Hyp-Gly-Clp-Hyp-Gly-Clp-Hyp-Gly-
Clp-Cys-Gly-Clp-Hyp-Gly-Clp-Hyp-Gly;
(SEQ ID NO: 132)
Clp-Hyp-Gly-Clp-Hyp-Gly-Clp-Hyp-Gly-Clp-Hyp-Gly-
Clp-Hyp-Gly-Cys-Hyp-Gly-Clp-Hyp-Gly;
(SEQ ID NO: 133)
Clp-Hyp-Gly-Clp-Hyp-Gly-Clp-Hyp-Gly-Clp-Hyp-Gly-
Clp-Hyp-Gly-Clp-Cys-Gly-Clp-Hyp-Gly;
(SEQ ID NO: 134)
Clp-Hyp-Gly-Clp-Hyp-Gly-Clp-Hyp-Gly-Clp-Hyp-Gly-
Clp-Hyp-Gly-Clp-Hyp-Gly-Cys-Hyp-Gly;
and
(SEQ ID NO: 135)
Clp-Hyp-Gly-Clp-Hyp-Gly-Clp-Hyp-Gly-Clp-Hyp-Gly-
Clp-Hyp-Gly-Clp-Hyp-Gly-Clp-Cys-Gly.

In certain other embodiments of the invention, the collagen mimetic peptide may comprise, consist of or have an amino acid sequence that is or corresponds to a 21-mer of any one of SEQ ID NOs:1-9, in which at least one methionine (Met) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 136)
Pro-Pro-Gly-Pro-Pro-Gly-Met-Pro-Gly-Pro-Pro-Gly-
Pro-Pro-Gly-Pro-Pro-Gly-Pro-Pro-Gly;
(SEQ ID NO: 137)
Hyp-Pro-Gly-Hyp-Pro-Gly-Met-Pro-Gly-Hyp-Pro-Gly-
Hyp-Pro-Gly-Hyp-Pro-Gly-Hyp-Pro-Gly;
(SEQ ID NO: 138)
Pro-Hyp-Gly-Pro-Hyp-Gly-Pro-Met-Gly-Pro-Hyp-Gly-
Pro-Hyp-Gly-Pro-Hyp-Gly-Pro-Hyp-Gly;
(SEQ ID NO: 139)
Pro-Flp-Gly-Pro-Flp-Gly-Pro-Met-Gly-Pro-Flp-Gly-
Pro-Flp-Gly-Pro-Flp-Gly-Pro-Flp-Gly;
(SEQ ID NO: 140)
Pro-Clp-Gly-Pro-Clp-Gly-Pro-Met-Gly-Pro-Clp-Gly-
Pro-Clp-Gly-Pro-Clp-Gly-Pro-Clp-Gly;
(SEQ ID NO: 141)
Met-Pro-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 142)
Pro-Met-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 143)
Pro-Pro-Gly-Met-Pro-Gly-Pro-Pro-Gly-Pro-Pro-Gly-
Pro-Pro-Gly-Pro-Pro-Gly-Pro-Pro-Gly;
(SEQ ID NO: 144)
Pro-Pro-Gly-Pro-Met-Gly-Pro-Pro-Gly-Pro-Pro-Gly-
Pro-Pro-Gly-Pro-Pro-Gly-Pro-Pro-Gly;
(SEQ ID NO: 145)
Pro-Pro-Gly-Pro-Pro-Gly-Pro-Met-Gly-Pro-Pro-Gly-
Pro-Pro-Gly-Pro-Pro-Gly-Pro-Pro-Gly;
(SEQ ID NO: 146)
Pro-Pro-Gly-Pro-Pro-Gly-Pro-Pro-Gly-Met-Pro-Gly-
Pro-Pro-Gly-Pro-Pro-Gly-Pro-Pro-Gly;
(SEQ ID NO: 147)
Pro-Pro-Gly-Pro-Pro-Gly-Pro-Pro-Gly-Pro-Met-Gly-
Pro-Pro-Gly-Pro-Pro-Gly-Pro-Pro-Gly;
(SEQ ID NO: 148)
Pro-Pro-Gly-Pro-Pro-Gly-Pro-Pro-Gly-Pro-Pro-Gly-
Met-Pro-Gly-Pro-Pro-Gly-Pro-Pro-Gly;
(SEQ ID NO: 149)
Pro-Pro-Gly-Pro-Pro-Gly-Pro-Pro-Gly-Pro-Pro-Gly-
Pro-Met-Gly-Pro-Pro-Gly-Pro-Pro-Gly;
(SEQ ID NO: 150)
Pro-Pro-Gly-Pro-Pro-Gly-Pro-Pro-Gly-Pro-Pro-Gly-
Pro-Pro-Gly-Met-Pro-Gly-Pro-Pro-Gly;
(SEQ ID NO: 151)
Pro-Pro-Gly-Pro-Pro-Gly-Pro-Pro-Gly-Pro-Pro-Gly-
Pro-Pro-Gly-Pro-Met-Gly-Pro-Pro-Gly;
(SEQ ID NO: 152)
Pro-Pro-Gly-Pro-Pro-Gly-Pro-Pro-Gly-Pro-Pro-Gly-
Pro-Pro-Gly-Pro-Pro-Gly-Met-Pro-Gly;
(SEQ ID NO: 153)
Pro-Pro-Gly-Pro-Pro-Gly-Pro-Pro-Gly-Pro-Pro-Gly-
Pro-Pro-Gly-Pro-Pro-Gly-Pro-Met-Gly;
(SEQ ID NO: 154)
Met-Pro-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 155)
Hyp-Met-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 156)
Hyp-Pro-Gly-Met-Pro-Gly-Hyp-Pro-Gly-Hyp-Pro-Gly-
Hyp-Pro-Gly-Hyp-Pro-Gly-Hyp-Pro-Gly;
(SEQ ID NO: 157)
Hyp-Pro-Gly-Hyp-Met-Gly-Hyp-Pro-Gly-Hyp-Pro-Gly-
Hyp-Pro-Gly-Hyp-Pro-Gly-Hyp-Pro-Gly;
(SEQ ID NO: 158)
Hyp-Pro-Gly-Hyp-Pro-Gly-Hyp-Met-Gly-Hyp-Pro-Gly-
Hyp-Pro-Gly-Hyp-Pro-Gly-Hyp-Pro-Gly;
(SEQ ID NO: 159)
Hyp-Pro-Gly-Hyp-Pro-Gly-Hyp-Pro-Gly-Met-Pro-Gly-
Hyp-Pro-Gly-Hyp-Pro-Gly-Hyp-Pro-Gly;
(SEQ ID NO: 160)
Hyp-Pro-Gly-Hyp-Pro-Gly-Hyp-Pro-Gly-Hyp-Met-Gly-
Hyp-Pro-Gly-Hyp-Pro-Gly-Hyp-Pro-Gly;
(SEQ ID NO: 161)
Hyp-Pro-Gly-Hyp-Pro-Gly-Hyp-Pro-Gly-Hyp-Pro-Gly-
Met-Pro-Gly-Hyp-Pro-Gly-Hyp-Pro-Gly;
(SEQ ID NO: 162)
Hyp-Pro-Gly-Hyp-Pro-Gly-Hyp-Pro-Gly-Hyp-Pro-Gly-
Hyp-Met-Gly-Hyp-Pro-Gly-Hyp-Pro-Gly;
(SEQ ID NO: 163)
Hyp-Pro-Gly-Hyp-Pro-Gly-Hyp-Pro-Gly-Hyp-Pro-Gly-
Hyp-Pro-Gly-Met-Pro-Gly-Hyp-Pro-Gly;
(SEQ ID NO: 164)
Hyp-Pro-Gly-Hyp-Pro-Gly-Hyp-Pro-Gly-Hyp-Pro-Gly-
Hyp-Pro-Gly-Hyp-Met-Gly-Hyp-Pro-Gly;
(SEQ ID NO: 165)
Hyp-Pro-Gly-Hyp-Pro-Gly-Hyp-Pro-Gly-Hyp-Pro-Gly-
Hyp-Pro-Gly-Hyp-Pro-Gly-Met-Pro-Gly;
(SEQ ID NO: 166)
Hyp-Pro-Gly-Hyp-Pro-Gly-Hyp-Pro-Gly-Hyp-Pro-Gly-
Hyp-Pro-Gly-Hyp-Pro-Gly-Hyp-Met-Gly;
(SEQ ID NO: 167)
Met-Hyp-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 168)
Pro-Met-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 169)
Pro-Hyp-Gly-Met-Hyp-Gly-Pro-Hyp-Gly-Pro-Hyp-Gly-
Pro-Hyp-Gly-Pro-Hyp-Gly-Pro-Hyp-Gly;
(SEQ ID NO: 170)
Pro-Hyp-Gly-Pro-Met-Gly-Pro-Hyp-Gly-Pro-Hyp-Gly-
Pro-Hyp-Gly-Pro-Hyp-Gly-Pro-Hyp-Gly;
(SEQ ID NO: 171)
Pro-Hyp-Gly-Pro-Hyp-Gly-Met-Hyp-Gly-Pro-Hyp-Gly-
Pro-Hyp-Gly-Pro-Hyp-Gly-Pro-Hyp-Gly;
(SEQ ID NO: 172)
Pro-Hyp-Gly-Pro-Hyp-Gly-Pro-Hyp-Gly-Met-Hyp-Gly-
Pro-Hyp-Gly-Pro-Hyp-Gly-Pro-Hyp-Gly;
(SEQ ID NO: 173)
Pro-Hyp-Gly-Pro-Hyp-Gly-Pro-Hyp-Gly-Pro-Met-Gly-
Pro-Hyp-Gly-Pro-Hyp-Gly-Pro-Hyp-Gly;
(SEQ ID NO: 174)
Pro-Hyp-Gly-Pro-Hyp-Gly-Pro-Hyp-Gly-Pro-Hyp-Gly-
Met-Hyp-Gly-Pro-Hyp-Gly-Pro-Hyp-Gly;
(SEQ ID NO: 175)
Pro-Hyp-Gly-Pro-Hyp-Gly-Pro-Hyp-Gly-Pro-Hyp-Gly-
Pro-Met-Gly-Pro-Hyp-Gly-Pro-Hyp-Gly;
(SEQ ID NO: 176)
Pro-Hyp-Gly-Pro-Hyp-Gly-Pro-Hyp-Gly-Pro-Hyp-Gly-
Pro-Hyp-Gly-Met-Hyp-Gly-Pro-Hyp-Gly;
(SEQ ID NO: 177)
Pro-Hyp-Gly-Pro-Hyp-Gly-Pro-Hyp-Gly-Pro-Hyp-Gly-
Pro-Hyp-Gly-Pro-Met-Gly-Pro-Hyp-Gly;
(SEQ ID NO: 178)
Pro-Hyp-Gly-Pro-Hyp-Gly-Pro-Hyp-Gly-Pro-Hyp-Gly-
Pro-Hyp-Gly-Pro-Hyp-Gly-Met-Hyp-Gly;
(SEQ ID NO: 179)
Pro-Hyp-Gly-Pro-Hyp-Gly-Pro-Hyp-Gly-Pro-Hyp-Gly-
Pro-Hyp-Gly-Pro-Hyp-Gly-Pro-Met-Gly;
(SEQ ID NO: 180)
Met-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 181)
Pro-Met-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 182)
Pro-Flp-Gly-Met-Flp-Gly-Pro-Flp-Gly-Pro-Flp-Gly-
Pro-Flp-Gly-Pro-Flp-Gly-Pro-Flp-Gly;
(SEQ ID NO: 183)
Pro-Flp-Gly-Pro-Met-Gly-Pro-Flp-Gly-Pro-Flp-Gly-
Pro-Flp-Gly-Pro-Flp-Gly-Pro-Flp-Gly;
(SEQ ID NO: 184)
Pro-Flp-Gly-Pro-Flp-Gly-Met-Flp-Gly-Pro-Flp-Gly-
Pro-Flp-Gly-Pro-Flp-Gly-Pro-Flp-Gly;
(SEQ ID NO: 185)
Pro-Flp-Gly-Pro-Flp-Gly-Pro-Flp-Gly-Met-Flp-Gly-
Pro-Flp-Gly-Pro-Flp-Gly-Pro-Flp-Gly;
(SEQ ID NO: 186)
Pro-Flp-Gly-Pro-Flp-Gly-Pro-Flp-Gly-Pro-Met-Gly-
Pro-Flp-Gly-Pro-Flp-Gly-Pro-Flp-Gly;
(SEQ ID NO: 187)
Pro-Flp-Gly-Pro-Flp-Gly-Pro-Flp-Gly-Pro-Flp-Gly-
Met-Flp-Gly-Pro-Flp-Gly-Pro-Flp-Gly;
(SEQ ID NO: 188)
Pro-Flp-Gly-Pro-Flp-Gly-Pro-Flp-Gly-Pro-Flp-Gly-
Pro-Met-Gly-Pro-Flp-Gly-Pro-Flp-Gly;
(SEQ ID NO: 189)
Pro-Flp-Gly-Pro-Flp-Gly-Pro-Flp-Gly-Pro-Flp-Gly-
Pro-Flp-Gly-Met-Flp-Gly-Pro-Flp-Gly;
(SEQ ID NO: 190)
Pro-Flp-Gly-Pro-Flp-Gly-Pro-Flp-Gly-Pro-Flp-Gly-
Pro-Flp-Gly-Pro-Met-Gly-Pro-Flp-Gly;
(SEQ ID NO: 191)
Pro-Flp-Gly-Pro-Flp-Gly-Pro-Flp-Gly-Pro-Flp-Gly-
Pro-Flp-Gly-Pro-Flp-Gly-Met-Flp-Gly;
(SEQ ID NO: 192)
Pro-Flp-Gly-Pro-Flp-Gly-Pro-Flp-Gly-Pro-Flp-Gly-
Pro-Flp-Gly-Pro-Flp-Gly-Pro-Met-Gly;
(SEQ ID NO: 193)
Met-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 194)
Pro-Met-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 195)
Pro-Flp-Gly-Met-Flp-Gly-Pro-Flp-Gly-Pro-Flp-Gly-
Pro-Flp-Gly-Pro-Flp-Gly-Pro-Flp-Gly;
(SEQ ID NO: 196)
Pro-Flp-Gly-Pro-Met-Gly-Pro-Flp-Gly-Pro-Flp-Gly-
Pro-Flp-Gly-Pro-Flp-Gly-Pro-Flp-Gly;
(SEQ ID NO: 197)
Pro-Flp-Gly-Pro-Flp-Gly-Met-Flp-Gly-Pro-Flp-Gly-
Pro-Flp-Gly-Pro-Flp-Gly-Pro-Flp-Gly;
(SEQ ID NO: 198)
Pro-Flp-Gly-Pro-Flp-Gly-Pro-Met-Gly-Pro-Flp-Gly-
Pro-Flp-Gly-Pro-Flp-Gly-Pro-Flp-Gly;
(SEQ ID NO: 199)
Pro-Flp-Gly-Pro-Flp-Gly-Pro-Flp-Gly-Met-Flp-Gly-
Pro-Flp-Gly-Pro-Flp-Gly-Pro-Flp-Gly;
(SEQ ID NO: 200)
Pro-Flp-Gly-Pro-Flp-Gly-Pro-Flp-Gly-Pro-Met-Gly-
Pro-Flp-Gly-Pro-Flp-Gly-Pro-Flp-Gly;
(SEQ ID NO: 201)
Pro-Flp-Gly-Pro-Flp-Gly-Pro-Flp-Gly-Pro-Flp-Gly-
Met-Flp-Gly-Pro-Flp-Gly-Pro-Flp-Gly;
(SEQ ID NO: 202)
Pro-Flp-Gly-Pro-Flp-Gly-Pro-Flp-Gly-Pro-Flp-Gly-
Pro-Met-Gly-Pro-Flp-Gly-Pro-Flp-Gly;
(SEQ ID NO: 203)
Pro-Flp-Gly-Pro-Flp-Gly-Pro-Flp-Gly-Pro-Flp-Gly-
Pro-Flp-Gly-Met-Flp-Gly-Pro-Flp-Gly;
(SEQ ID NO: 204)
Pro-Flp-Gly-Pro-Flp-Gly-Pro-Flp-Gly-Pro-Flp-Gly-
Pro-Flp-Gly-Pro-Met-Gly-Pro-Flp-Gly;
(SEQ ID NO: 205)
Pro-Flp-Gly-Pro-Flp-Gly-Pro-Flp-Gly-Pro-Flp-Gly-
Pro-Flp-Gly-Pro-Flp-Gly-Met-Flp-Gly;
(SEQ ID NO: 206)
Pro-Flp-Gly-Pro-Flp-Gly-Pro-Flp-Gly-Pro-Flp-Gly-
Pro-Flp-Gly-Pro-Flp-Gly-Pro-Met-Gly;
(SEQ ID NO: 207)
Met-Hyp-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 208)
Flp-Met-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 209)
Flp-Hyp-Gly-Met-Hyp-Gly-Flp-Hyp-Gly-Flp-Hyp-Gly-
Flp-Hyp-Gly-Flp-Hyp-Gly-Flp-Hyp-Gly;
(SEQ ID NO: 210)
Flp-Hyp-Gly-Flp-Met-Gly-Flp-Hyp-Gly-Flp-Hyp-Gly-
Flp-Hyp-Gly-Flp-Hyp-Gly-Flp-Hyp-Gly;
(SEQ ID NO: 211)
Flp-Hyp-Gly-Flp-Hyp-Gly-Met-Hyp-Gly-Flp-Hyp-Gly-
Flp-Hyp-Gly-Flp-Hyp-Gly-Flp-Hyp-Gly;
(SEQ ID NO: 212)
Flp-Hyp-Gly-Flp-Hyp-Gly-Flp-Met-Gly-Flp-Hyp-Gly-
Flp-Hyp-Gly-Flp-Hyp-Gly-Flp-Hyp-Gly;
(SEQ ID NO: 213)
Flp-Hyp-Gly-Flp-Hyp-Gly-Flp-Hyp-Gly-Met-Hyp-Gly-
Flp-Hyp-Gly-Flp-Hyp-Gly-Flp-Hyp-Gly;
(SEQ ID NO: 214)
Flp-Hyp-Gly-Flp-Hyp-Gly-Flp-Hyp-Gly-Flp-Met-Gly-
Flp-Hyp-Gly-Flp-Hyp-Gly-Flp-Hyp-Gly;
(SEQ ID NO: 215)
Flp-Hyp-Gly-Flp-Hyp-Gly-Flp-Hyp-Gly-Flp-Hyp-Gly-
Met-Hyp-Gly-Flp-Hyp-Gly-Flp-Hyp-Gly;
(SEQ ID NO: 216)
Flp-Hyp-Gly-Flp-Hyp-Gly-Flp-Hyp-Gly-Flp-Hyp-Gly-
Flp-Met-Gly-Flp-Hyp-Gly-Flp-Hyp-Gly;
(SEQ ID NO: 217)
Flp-Hyp-Gly-Flp-Hyp-Gly-Flp-Hyp-Gly-Flp-Hyp-Gly-
Flp-Hyp-Gly-Met-Hyp-Gly-Flp-Hyp-Gly;
(SEQ ID NO: 218)
Flp-Hyp-Gly-Flp-Hyp-Gly-Flp-Hyp-Gly-Flp-Hyp-Gly-
Flp-Hyp-Gly-Flp-Met-Gly-Flp-Hyp-Gly;
(SEQ ID NO: 219)
Flp-Hyp-Gly-Flp-Hyp-Gly-Flp-Hyp-Gly-Flp-Hyp-Gly-
Flp-Hyp-Gly-Flp-Hyp-Gly-Met-Hyp-Gly;
(SEQ ID NO: 220)
Flp-Hyp-Gly-Flp-Hyp-Gly-Flp-Hyp-Gly-Flp-Hyp-Gly-
Flp-Hyp-Gly-Flp-Hyp-Gly-Flp-Met-Gly;
(SEQ ID NO: 221)
Met-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 222)
Pro-Met-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 223)
Pro-Clp-Gly-Met-Clp-Gly-Pro-Clp-Gly-Pro-Clp-Gly-
Pro-Clp-Gly-Pro-Clp-Gly-Pro-Clp-Gly;
(SEQ ID NO: 224)
Pro-Clp-Gly-Pro-Met-Gly-Pro-Clp-Gly-Pro-Clp-Gly-
Pro-Clp-Gly-Pro-Clp-Gly-Pro-Clp-Gly;
(SEQ ID NO: 225)
Pro-Clp-Gly-Pro-Clp-Gly-Met-Clp-Gly-Pro-Clp-Gly-
Pro-Clp-Gly-Pro-Clp-Gly-Pro-Clp-Gly;
(SEQ ID NO: 226)
Pro-Clp-Gly-Pro-Clp-Gly-Pro-Clp-Gly-Met-Clp-Gly-
Pro-Clp-Gly-Pro-Clp-Gly-Pro-Clp-Gly;
(SEQ ID NO: 227)
Pro-Clp-Gly-Pro-Clp-Gly-Pro-Clp-Gly-Pro-Met-Gly-
Pro-Clp-Gly-Pro-Clp-Gly-Pro-Clp-Gly;
(SEQ ID NO: 228)
Pro-Clp-Gly-Pro-Clp-Gly-Pro-Clp-Gly-Pro-Clp-Gly-
Met-Clp-Gly-Pro-Clp-Gly-Pro-Clp-Gly;
(SEQ ID NO: 229)
Pro-Clp-Gly-Pro-Clp-Gly-Pro-Clp-Gly-Pro-Clp-Gly-
Pro-Met-Gly-Pro-Clp-Gly-Pro-Clp-Gly;
(SEQ ID NO: 230)
Pro-Clp-Gly-Pro-Clp-Gly-Pro-Clp-Gly-Pro-Clp-Gly-
Pro-Clp-Gly-Met-Clp-Gly-Pro-Clp-Gly;
(SEQ ID NO: 231)
Pro-Clp-Gly-Pro-Clp-Gly-Pro-Clp-Gly-Pro-Clp-Gly-
Pro-Clp-Gly-Pro-Met-Gly-Pro-Clp-Gly;
(SEQ ID NO: 232)
Pro-Clp-Gly-Pro-Clp-Gly-Pro-Clp-Gly-Pro-Clp-Gly-
Pro-Clp-Gly-Pro-Clp-Gly-Met-Clp-Gly;
(SEQ ID NO: 233)
Pro-Clp-Gly-Pro-Clp-Gly-Pro-Clp-Gly-Pro-Clp-Gly-
Pro-Clp-Gly-Pro-Clp-Gly-Pro-Met-Gly;
(SEQ ID NO: 234)
Met-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 235)
Pro-Met-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 236)
Pro-Clp-Gly-Met-Clp-Gly-Pro-Clp-Gly-Pro-Clp-Gly-
Pro-Clp-Gly-Pro-Clp-Gly-Pro-Clp-Gly;
(SEQ ID NO: 237)
Pro-Clp-Gly-Pro-Met-Gly-Pro-Clp-Gly-Pro-Clp-Gly-
Pro-Clp-Gly-Pro-Clp-Gly-Pro-Clp-Gly;
(SEQ ID NO: 238)
Pro-Clp-Gly-Pro-Clp-Gly-Met-Clp-Gly-Pro-Clp-Gly-
Pro-Clp-Gly-Pro-Clp-Gly-Pro-Clp-Gly;
(SEQ ID NO: 239)
Pro-Clp-Gly-Pro-Clp-Gly-Pro-Met-Gly-Pro-Clp-Gly-
Pro-Clp-Gly-Pro-Clp-Gly-Pro-Clp-Gly;
(SEQ ID NO: 240)
Pro-Clp-Gly-Pro-Clp-Gly-Pro-Clp-Gly-Met-Clp-Gly-
Pro-Clp-Gly-Pro-Clp-Gly-Pro-Clp-Gly;
(SEQ ID NO: 241)
Pro-Clp-Gly-Pro-Clp-Gly-Pro-Clp-Gly-Pro-Met-Gly-
Pro-Clp-Gly-Pro-Clp-Gly-Pro-Clp-Gly;
(SEQ ID NO: 242)
Pro-Clp-Gly-Pro-Clp-Gly-Pro-Clp-Gly-Pro-Clp-Gly-
Met-Clp-Gly-Pro-Clp-Gly-Pro-Clp-Gly;
(SEQ ID NO: 243)
Pro-Clp-Gly-Pro-Clp-Gly-Pro-Clp-Gly-Pro-Clp-Gly-
Pro-Met-Gly-Pro-Clp-Gly-Pro-Clp-Gly;
(SEQ ID NO: 244)
Pro-Clp-Gly-Pro-Clp-Gly-Pro-Clp-Gly-Pro-Clp-Gly-
Pro-Clp-Gly-Met-Clp-Gly-Pro-Clp-Gly;
(SEQ ID NO: 245)
Pro-Clp-Gly-Pro-Clp-Gly-Pro-Clp-Gly-Pro-Clp-Gly-
Pro-Clp-Gly-Pro-Met-Gly-Pro-Clp-Gly;
(SEQ ID NO: 246)
Pro-Clp-Gly-Pro-Clp-Gly-Pro-Clp-Gly-Pro-Clp-Gly-
Pro-Clp-Gly-Pro-Clp-Gly-Met-Clp-Gly;
(SEQ ID NO: 247)
Pro-Clp-Gly-Pro-Clp-Gly-Pro-Clp-Gly-Pro-Clp-Gly-
Pro-Clp-Gly-Pro-Clp-Gly-Pro-Met-Gly;
(SEQ ID NO: 248)
Met-Hyp-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 249)
Clp-Met-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 250)
Clp-Hyp-Gly-Met-Hyp-Gly-Clp-Hyp-Gly-Clp-Hyp-Gly-
Clp-Hyp-Gly-Clp-Hyp-Gly-Clp-Hyp-Gly;
(SEQ ID NO: 251)
Clp-Hyp-Gly-Clp-Met-Gly-Clp-Hyp-Gly-Clp-Hyp-Gly-
Clp-Hyp-Gly-Clp-Hyp-Gly-Clp-Hyp-Gly;
(SEQ ID NO: 252)
Clp-Hyp-Gly-Clp-Hyp-Gly-Met-Hyp-Gly-Clp-Hyp-Gly-
Clp-Hyp-Gly-Clp-Hyp-Gly-Clp-Hyp-Gly;
(SEQ ID NO: 253)
Clp-Hyp-Gly-Clp-Hyp-Gly-Clp-Met-Gly-Clp-Hyp-Gly-
Clp-Hyp-Gly-Clp-Hyp-Gly-Clp-Hyp-Gly;
(SEQ ID NO: 254)
Clp-Hyp-Gly-Clp-Hyp-Gly-Clp-Hyp-Gly-Met-Hyp-Gly-
Clp-Hyp-Gly-Clp-Hyp-Gly-Clp-Hyp-Gly;
(SEQ ID NO: 255)
Clp-Hyp-Gly-Clp-Hyp-Gly-Clp-Hyp-Gly-Clp-Met-Gly-
Clp-Hyp-Gly-Clp-Hyp-Gly-Clp-Hyp-Gly;
(SEQ ID NO: 256)
Clp-Hyp-Gly-Clp-Hyp-Gly-Clp-Hyp-Gly-Clp-Hyp-Gly-
Met-Hyp-Gly-Clp-Hyp-Gly-Clp-Hyp-Gly;
(SEQ ID NO: 257)
Clp-Hyp-Gly-Clp-Hyp-Gly-Clp-Hyp-Gly-Clp-Hyp-Gly-
Clp-Met-Gly-Clp-Hyp-Gly-Clp-Hyp-Gly;
(SEQ ID NO: 258)
Clp-Hyp-Gly-Clp-Hyp-Gly-Clp-Hyp-Gly-Clp-Hyp-Gly-
Clp-Hyp-Gly-Met-Hyp-Gly-Clp-Hyp-Gly;
(SEQ ID NO: 259)
Clp-Hyp-Gly-Clp-Hyp-Gly-Clp-Hyp-Gly-Clp-Hyp-Gly-
Clp-Hyp-Gly-Clp-Met-Gly-Clp-Hyp-Gly;
(SEQ ID NO: 260)
Clp-Hyp-Gly-Clp-Hyp-Gly-Clp-Hyp-Gly-Clp-Hyp-Gly-
Clp-Hyp-Gly-Clp-Hyp-Gly-Met-Hyp-Gly;
and
(SEQ ID NO: 261)
Clp-Hyp-Gly-Clp-Hyp-Gly-Clp-Hyp-Gly-Clp-Hyp-Gly-
Clp-Hyp-Gly-Clp-Hyp-Gly-Clp-Met-Gly.

In certain other embodiments of the invention, the collagen mimetic peptide may comprise, consist of or have an amino acid sequence that is or corresponds to a 21-mer of any one of SEQ ID NOs:1-9, in which at least one lysine (Lys) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 262)
Pro-Pro-Gly-Pro-Pro-Gly-Lys-Pro-Gly-Pro-Pro-Gly-
Pro-Pro-Gly-Pro-Pro-Gly-Pro-Pro-Gly;
(SEQ ID NO: 263)
Hyp-Pro-Gly-Hyp-Pro-Gly-Lys-Pro-Gly-Hyp-Pro-Gly-
Hyp-Pro-Gly-Hyp-Pro-Gly-Hyp-Pro-Gly;
(SEQ ID NO: 264)
Pro-Hyp-Gly-Pro-Hyp-Gly-Pro-Lys-Gly-Pro-Hyp-Gly-
Pro-Hyp-Gly-Pro-Hyp-Gly-Pro-Hyp-Gly;
(SEQ ID NO: 265)
Pro-Flp-Gly-Pro-Flp-Gly-Pro-Lys-Gly-Pro-Flp-Gly-
Pro-Flp-Gly-Pro-Flp-Gly-Pro-Flp-Gly;
(SEQ ID NO: 266)
Pro-Clp-Gly-Pro-Clp-Gly-Pro-Lys-Gly-Pro-Clp-Gly-
Pro-Clp-Gly-Pro-Clp-Gly-Pro-Clp-Gly;
(SEQ ID NO: 267)
Lys-Pro-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 268)
Pro-Lys-Gly-Pro-Pro-Gly-Pro-Pro-Gly-Pro-Pro-Gly-
Pro-Pro-Gly-Pro-Pro-Gly-Pro-Pro-Gly;
(SEQ ID NO: 269)
Pro-Pro-Gly-Lys-Pro-Gly-Pro-Pro-Gly-Pro-Pro-Gly-
Pro-Pro-Gly-Pro-Pro-Gly-Pro-Pro-Gly;
(SEQ ID NO: 270)
Pro-Pro-Gly-Pro-Lys-Gly-Pro-Pro-Gly-Pro-Pro-Gly-
Pro-Pro-Gly-Pro-Pro-Gly-Pro-Pro-Gly;
(SEQ ID NO: 271)
Pro-Pro-Gly-Pro-Pro-Gly-Pro-Lys-Gly-Pro-Pro-Gly-
Pro-Pro-Gly-Pro-Pro-Gly-Pro-Pro-Gly;
(SEQ ID NO: 272)
Pro-Pro-Gly-Pro-Pro-Gly-Pro-Pro-Gly-Lys-Pro-Gly-
Pro-Pro-Gly-Pro-Pro-Gly-Pro-Pro-Gly;
(SEQ ID NO: 273)
Pro-Pro-Gly-Pro-Pro-Gly-Pro-Pro-Gly-Pro-Lys-Gly-
Pro-Pro-Gly-Pro-Pro-Gly-Pro-Pro-Gly;
(SEQ ID NO: 274)
Pro-Pro-Gly-Pro-Pro-Gly-Pro-Pro-Gly-Pro-Pro-Gly-
Lys-Pro-Gly-Pro-Pro-Gly-Pro-Pro-Gly;
(SEQ ID NO: 275)
Pro-Pro-Gly-Pro-Pro-Gly-Pro-Pro-Gly-Pro-Pro-Gly-
Pro-Lys-Gly-Pro-Pro-Gly-Pro-Pro-Gly;
(SEQ ID NO: 276)
Pro-Pro-Gly-Pro-Pro-Gly-Pro-Pro-Gly-Pro-Pro-Gly-
Pro-Pro-Gly-Lys-Pro-Gly-Pro-Pro-Gly;
(SEQ ID NO: 277)
Pro-Pro-Gly-Pro-Pro-Gly-Pro-Pro-Gly-Pro-Pro-Gly-
Pro-Pro-Gly-Pro-Lys-Gly-Pro-Pro-Gly;
(SEQ ID NO: 278)
Pro-Pro-Gly-Pro-Pro-Gly-Pro-Pro-Gly-Pro-Pro-Gly-
Pro-Pro-Gly-Pro-Pro-Gly-Lys-Pro-Gly;
(SEQ ID NO: 279)
Pro-Pro-Gly-Pro-Pro-Gly-Pro-Pro-Gly-Pro-Pro-Gly-
Pro-Pro-Gly-Pro-Pro-Gly-Pro-Lys-Gly;
(SEQ ID NO: 280)
Lys-Pro-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 281)
Hyp-Lys-Gly-Hyp-Pro-Gly-Hyp-Pro-Gly-Hyp-Pro-Gly-
Hyp-Pro-Gly-Hyp-Pro-Gly-Hyp-Pro-Gly;
(SEQ ID NO: 282)
Hyp-Pro-Gly-Lys-Pro-Gly-Hyp-Pro-Gly-Hyp-Pro-Gly-
Hyp-Pro-Gly-Hyp-Pro-Gly-Hyp-Pro-Gly;
(SEQ ID NO: 283)
Hyp-Pro-Gly-Hyp-Lys-Gly-Hyp-Pro-Gly-Hyp-Pro-Gly-
Hyp-Pro-Gly-Hyp-Pro-Gly-Hyp-Pro-Gly;
(SEQ ID NO: 284)
Hyp-Pro-Gly-Hyp-Pro-Gly-Hyp-Lys-Gly-Hyp-Pro-Gly-
Hyp-Pro-Gly-Hyp-Pro-Gly-Hyp-Pro-Gly;
(SEQ ID NO: 285)
Hyp-Pro-Gly-Hyp-Pro-Gly-Hyp-Pro-Gly-Lys-Pro-Gly-
Hyp-Pro-Gly-Hyp-Pro-Gly-Hyp-Pro-Gly;
(SEQ ID NO: 286)
Hyp-Pro-Gly-Hyp-Pro-Gly-Hyp-Pro-Gly-Hyp-Lys-Gly-
Hyp-Pro-Gly-Hyp-Pro-Gly-Hyp-Pro-Gly;
(SEQ ID NO: 287)
Hyp-Pro-Gly-Hyp-Pro-Gly-Hyp-Pro-Gly-Hyp-Pro-Gly-
Lys-Pro-Gly-Hyp-Pro-Gly-Hyp-Pro-Gly;
(SEQ ID NO: 288)
Hyp-Pro-Gly-Hyp-Pro-Gly-Hyp-Pro-Gly-Hyp-Pro-Gly-
Hyp-Lys-Gly-Hyp-Pro-Gly-Hyp-Pro-Gly;
(SEQ ID NO: 289)
Hyp-Pro-Gly-Hyp-Pro-Gly-Hyp-Pro-Gly-Hyp-Pro-Gly-
Hyp-Pro-Gly-Lys-Pro-Gly-Hyp-Pro-Gly;
(SEQ ID NO: 290)
Hyp-Pro-Gly-Hyp-Pro-Gly-Hyp-Pro-Gly-Hyp-Pro-Gly-
Hyp-Pro-Gly-Hyp-Lys-Gly-Hyp-Pro-Gly;
(SEQ ID NO: 291)
Hyp-Pro-Gly-Hyp-Pro-Gly-Hyp-Pro-Gly-Hyp-Pro-Gly-
Hyp-Pro-Gly-Hyp-Pro-Gly-Lys-Pro-Gly;
(SEQ ID NO: 292)
Hyp-Pro-Gly-Hyp-Pro-Gly-Hyp-Pro-Gly-Hyp-Pro-Gly-
Hyp-Pro-Gly-Hyp-Pro-Gly-Hyp-Lys-Gly;
(SEQ ID NO: 293)
Lys-Hyp-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 294)
Pro-Lys-Gly-Pro-Hyp-Gly-Pro-Hyp-Gly-Pro-Hyp-Gly-
Pro-Hyp-Gly-Pro-Hyp-Gly-Pro-Hyp-Gly;
(SEQ ID NO: 295)
Pro-Hyp-Gly-Lys-Hyp-Gly-Pro-Hyp-Gly-Pro-Hyp-Gly-
Pro-Hyp-Gly-Pro-Hyp-Gly-Pro-Hyp-Gly;
(SEQ ID NO: 296)
Pro-Hyp-Gly-Pro-Lys-Gly-Pro-Hyp-Gly-Pro-Hyp-Gly-
Pro-Hyp-Gly-Pro-Hyp-Gly-Pro-Hyp-Gly;
(SEQ ID NO: 297)
Pro-Hyp-Gly-Pro-Hyp-Gly-Lys-Hyp-Gly-Pro-Hyp-Gly-
Pro-Hyp-Gly-Pro-Hyp-Gly-Pro-Hyp-Gly;
(SEQ ID NO: 298)
Pro-Hyp-Gly-Pro-Hyp-Gly-Pro-Hyp-Gly-Lys-Hyp-Gly-
Pro-Hyp-Gly-Pro-Hyp-Gly-Pro-Hyp-Gly;
(SEQ ID NO: 299)
Pro-Hyp-Gly-Pro-Hyp-Gly-Pro-Hyp-Gly-Pro-Lys-Gly-
Pro-Hyp-Gly-Pro-Hyp-Gly-Pro-Hyp-Gly;
(SEQ ID NO: 300)
Pro-Hyp-Gly-Pro-Hyp-Gly-Pro-Hyp-Gly-Pro-Hyp-Gly-
Lys-Hyp-Gly-Pro-Hyp-Gly-Pro-Hyp-Gly;
(SEQ ID NO: 301)
Pro-Hyp-Gly-Pro-Hyp-Gly-Pro-Hyp-Gly-Pro-Hyp-Gly-
Pro-Lys-Gly-Pro-Hyp-Gly-Pro-Hyp-Gly;
(SEQ ID NO: 302)
Pro-Hyp-Gly-Pro-Hyp-Gly-Pro-Hyp-Gly-Pro-Hyp-Gly-
Pro-Hyp-Gly-Lys-Hyp-Gly-Pro-Hyp-Gly;
(SEQ ID NO: 303)
Pro-Hyp-Gly-Pro-Hyp-Gly-Pro-Hyp-Gly-Pro-Hyp-Gly-
Pro-Hyp-Gly-Pro-Lys-Gly-Pro-Hyp-Gly;
(SEQ ID NO: 304)
Pro-Hyp-Gly-Pro-Hyp-Gly-Pro-Hyp-Gly-Pro-Hyp-Gly-
Pro-Hyp-Gly-Pro-Hyp-Gly-Lys-Hyp-Gly;
(SEQ ID NO: 305)
Pro-Hyp-Gly-Pro-Hyp-Gly-Pro-Hyp-Gly-Pro-Hyp-Gly-
Pro-Hyp-Gly-Pro-Hyp-Gly-Pro-Lys-Gly;
(SEQ ID NO: 306)
Lys-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 307)
Pro-Lys-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 308)
Pro-Flp-Gly-Lys-Flp-Gly-Pro-Flp-Gly-Pro-Flp-Gly-
Pro-Flp-Gly-Pro-Flp-Gly-Pro-Flp-Gly;
(SEQ ID NO: 309)
Pro-Flp-Gly-Pro-Lys-Gly-Pro-Flp-Gly-Pro-Flp-Gly-
Pro-Flp-Gly-Pro-Flp-Gly-Pro-Flp-Gly;
(SEQ ID NO: 310)
Pro-Flp-Gly-Pro-Flp-Gly-Lys-Flp-Gly-Pro-Flp-Gly-
Pro-Flp-Gly-Pro-Flp-Gly-Pro-Flp-Gly;
(SEQ ID NO: 311)
Pro-Flp-Gly-Pro-Flp-Gly-Pro-Flp-Gly-Lys-Flp-Gly-
Pro-Flp-Gly-Pro-Flp-Gly-Pro-Flp-Gly;
(SEQ ID NO: 312)
Pro-Flp-Gly-Pro-Flp-Gly-Pro-Flp-Gly-Pro-Lys-Gly-
Pro-Flp-Gly-Pro-Flp-Gly-Pro-Flp-Gly;
(SEQ ID NO: 313)
Pro-Flp-Gly-Pro-Flp-Gly-Pro-Flp-Gly-Pro-Flp-Gly-
Lys-Flp-Gly-Pro-Flp-Gly-Pro-Flp-Gly;
(SEQ ID NO: 314)
Pro-Flp-Gly-Pro-Flp-Gly-Pro-Flp-Gly-Pro-Flp-Gly-
Pro-Lys-Gly-Pro-Flp-Gly-Pro-Flp-Gly;
(SEQ ID NO: 315)
Pro-Flp-Gly-Pro-Flp-Gly-Pro-Flp-Gly-Pro-Flp-Gly-
Pro-Flp-Gly-Lys-Flp-Gly-Pro-Flp-Gly;
(SEQ ID NO: 316)
Pro-Flp-Gly-Pro-Flp-Gly-Pro-Flp-Gly-Pro-Flp-Gly-
Pro-Flp-Gly-Pro-Lys-Gly-Pro-Flp-Gly;
(SEQ ID NO: 317)
Pro-Flp-Gly-Pro-Flp-Gly-Pro-Flp-Gly-Pro-Flp-Gly-
Pro-Flp-Gly-Pro-Flp-Gly-Lys-Flp-Gly;
(SEQ ID NO: 318)
Pro-Flp-Gly-Pro-Flp-Gly-Pro-Flp-Gly-Pro-Flp-Gly-
Pro-Flp-Gly-Pro-Flp-Gly-Pro-Lys-Gly;
(SEQ ID NO: 319)
Lys-Flp-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 320)
Pro-Lys-Gly-Pro-Flp-Gly-Pro-Flp-Gly-Pro-Flp-Gly-
Pro-Flp-Gly-Pro-Flp-Gly-Pro-Flp-Gly;
(SEQ ID NO: 321)
Pro-Flp-Gly-Lys-Flp-Gly-Pro-Flp-Gly-Pro-Flp-Gly-
Pro-Flp-Gly-Pro-Flp-Gly-Pro-Flp-Gly;
(SEQ ID NO: 322)
Pro-Flp-Gly-Pro-Lys-Gly-Pro-Flp-Gly-Pro-Flp-Gly-
Pro-Flp-Gly-Pro-Flp-Gly-Pro-Flp-Gly;
(SEQ ID NO: 323)
Pro-Flp-Gly-Pro-Flp-Gly-Lys-Flp-Gly-Pro-Flp-Gly-
Pro-Flp-Gly-Pro-Flp-Gly-Pro-Flp-Gly;
(SEQ ID NO: 324)
Pro-Flp-Gly-Pro-Flp-Gly-Pro-Lys-Gly-Pro-Flp-Gly-
Pro-Flp-Gly-Pro-Flp-Gly-Pro-Flp-Gly;
(SEQ ID NO: 325)
Pro-Flp-Gly-Pro-Flp-Gly-Pro-Flp-Gly-Lys-Flp-Gly-
Pro-Flp-Gly-Pro-Flp-Gly-Pro-Flp-Gly;
(SEQ ID NO: 326)
Pro-Flp-Gly-Pro-Flp-Gly-Pro-Flp-Gly-Pro-Lys-Gly-
Pro-Flp-Gly-Pro-Flp-Gly-Pro-Flp-Gly;
(SEQ ID NO: 327)
Pro-Flp-Gly-Pro-Flp-Gly-Pro-Flp-Gly-Pro-Flp-Gly-
Lys-Flp-Gly-Pro-Flp-Gly-Pro-Flp-Gly;
(SEQ ID NO: 328)
Pro-Flp-Gly-Pro-Flp-Gly-Pro-Flp-Gly-Pro-Flp-Gly-
Pro-Lys-Gly-Pro-Flp-Gly-Pro-Flp-Gly;
(SEQ ID NO: 329)
Pro-Flp-Gly-Pro-Flp-Gly-Pro-Flp-Gly-Pro-Flp-Gly-
Pro-Flp-Gly-Lys-Flp-Gly-Pro-Flp-Gly;
(SEQ ID NO: 330)
Pro-Flp-Gly-Pro-Flp-Gly-Pro-Flp-Gly-Pro-Flp-Gly-
Pro-Flp-Gly-Pro-Lys-Gly-Pro-Flp-Gly;
(SEQ ID NO: 331)
Pro-Flp-Gly-Pro-Flp-Gly-Pro-Flp-Gly-Pro-Flp-Gly-
Pro-Flp-Gly-Pro-Flp-Gly-Lys-Flp-Gly;
(SEQ ID NO: 332)
Pro-Flp-Gly-Pro-Flp-Gly-Pro-Flp-Gly-Pro-Flp-Gly-
Pro-Flp-Gly-Pro-Flp-Gly-Pro-Lys-Gly;
(SEQ ID NO: 333)
Lys-Hyp-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 334)
Flp-Lys-Gly-Flp-Hyp-Gly-Flp-Hyp-Gly-Flp-Hyp-Gly-
Flp-Hyp-Gly-Flp-Hyp-Gly-Flp-Hyp-Gly;
(SEQ ID NO: 335)
Flp-Hyp-Gly-Lys-Hyp-Gly-Flp-Hyp-Gly-Flp-Hyp-Gly-
Flp-Hyp-Gly-Flp-Hyp-Gly-Flp-Hyp-Gly;
(SEQ ID NO: 336)
Flp-Hyp-Gly-Flp-Lys-Gly-Flp-Hyp-Gly-Flp-Hyp-Gly-
Flp-Hyp-Gly-Flp-Hyp-Gly-Flp-Hyp-Gly;
(SEQ ID NO: 337)
Flp-Hyp-Gly-Flp-Hyp-Gly-Lys-Hyp-Gly-Flp-Hyp-Gly-
Flp-Hyp-Gly-Flp-Hyp-Gly-Flp-Hyp-Gly;
(SEQ ID NO: 338)
Flp-Hyp-Gly-Flp-Hyp-Gly-Flp-Lys-Gly-Flp-Hyp-Gly-
Flp-Hyp-Gly-Flp-Hyp-Gly-Flp-Hyp-Gly;
(SEQ ID NO: 339)
Flp-Hyp-Gly-Flp-Hyp-Gly-Flp-Hyp-Gly-Lys-Hyp-Gly-
Flp-Hyp-Gly-Flp-Hyp-Gly-Flp-Hyp-Gly;
(SEQ ID NO: 340)
Flp-Hyp-Gly-Flp-Hyp-Gly-Flp-Hyp-Gly-Flp-Lys-Gly-
Flp-Hyp-Gly-Flp-Hyp-Gly-Flp-Hyp-Gly;
(SEQ ID NO: 341)
Flp-Hyp-Gly-Flp-Hyp-Gly-Flp-Hyp-Gly-Flp-Hyp-Gly-
Lys-Hyp-Gly-Flp-Hyp-Gly-Flp-Hyp-Gly;
(SEQ ID NO: 342)
Flp-Hyp-Gly-Flp-Hyp-Gly-Flp-Hyp-Gly-Flp-Hyp-Gly-
Flp-Lys-Gly-Flp-Hyp-Gly-Flp-Hyp-Gly;
(SEQ ID NO: 343)
Flp-Hyp-Gly-Flp-Hyp-Gly-Flp-Hyp-Gly-Flp-Hyp-Gly-
Flp-Hyp-Gly-Lys-Hyp-Gly-Flp-Hyp-Gly;
(SEQ ID NO: 344)
Flp-Hyp-Gly-Flp-Hyp-Gly-Flp-Hyp-Gly-Flp-Hyp-Gly-
Flp-Hyp-Gly-Flp-Lys-Gly-Flp-Hyp-Gly;
(SEQ ID NO: 345)
Flp-Hyp-Gly-Flp-Hyp-Gly-Flp-Hyp-Gly-Flp-Hyp-Gly-
Flp-Hyp-Gly-Flp-Hyp-Gly-Lys-Hyp-Gly;
(SEQ ID NO: 346)
Flp-Hyp-Gly-Flp-Hyp-Gly-Flp-Hyp-Gly-Flp-Hyp-Gly-
Flp-Hyp-Gly-Flp-Hyp-Gly-Flp-Lys-Gly;
(SEQ ID NO: 347)
Lys-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 348)
Pro-Lys-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 349)
Pro-Clp-Gly-Lys-Clp-Gly-Pro-Clp-Gly-Pro-Clp-Gly-
Pro-Clp-Gly-Pro-Clp-Gly-Pro-Clp-Gly;
(SEQ ID NO: 350)
Pro-Clp-Gly-Pro-Lys-Gly-Pro-Clp-Gly-Pro-Clp-Gly-
Pro-Clp-Gly-Pro-Clp-Gly-Pro-Clp-Gly;
(SEQ ID NO: 351)
Pro-Clp-Gly-Pro-Clp-Gly-Lys-Clp-Gly-Pro-Clp-Gly-
Pro-Clp-Gly-Pro-Clp-Gly-Pro-Clp-Gly;
(SEQ ID NO: 352)
Pro-Clp-Gly-Pro-Clp-Gly-Pro-Clp-Gly-Lys-Clp-Gly-
Pro-Clp-Gly-Pro-Clp-Gly-Pro-Clp-Gly;
(SEQ ID NO: 353)
Pro-Clp-Gly-Pro-Clp-Gly-Pro-Clp-Gly-Pro-Lys-Gly-
Pro-Clp-Gly-Pro-Clp-Gly-Pro-Clp-Gly;
(SEQ ID NO: 354)
Pro-Clp-Gly-Pro-Clp-Gly-Pro-Clp-Gly-Pro-Clp-Gly-
Lys-Clp-Gly-Pro-Clp-Gly-Pro-Clp-Gly;
(SEQ ID NO: 355)
Pro-Clp-Gly-Pro-Clp-Gly-Pro-Clp-Gly-Pro-Clp-Gly-
Pro-Lys-Gly-Pro-Clp-Gly-Pro-Clp-Gly;
(SEQ ID NO: 356)
Pro-Clp-Gly-Pro-Clp-Gly-Pro-Clp-Gly-Pro-Clp-Gly-
Pro-Clp-Gly-Lys-Clp-Gly-Pro-Clp-Gly;
(SEQ ID NO: 357)
Pro-Clp-Gly-Pro-Clp-Gly-Pro-Clp-Gly-Pro-Clp-Gly-
Pro-Clp-Gly-Pro-Lys-Gly-Pro-Clp-Gly;
(SEQ ID NO: 358)
Pro-Clp-Gly-Pro-Clp-Gly-Pro-Clp-Gly-Pro-Clp-Gly-
Pro-Clp-Gly-Pro-Clp-Gly-Lys-Clp-Gly;
(SEQ ID NO: 359)
Pro-Clp-Gly-Pro-Clp-Gly-Pro-Clp-Gly-Pro-Clp-Gly-
Pro-Clp-Gly-Pro-Clp-Gly-Pro-Lys-Gly;
(SEQ ID NO: 360)
Lys-Clp-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 361)
Pro-Lys-Gly-Pro-Clp-Gly-Pro-Clp-Gly-Pro-Clp-Gly-
Pro-Clp-Gly-Pro-Clp-Gly-Pro-Clp-Gly;
(SEQ ID NO: 362)
Pro-Clp-Gly-Lys-Clp-Gly-Pro-Clp-Gly-Pro-Clp-Gly-
Pro-Clp-Gly-Pro-Clp-Gly-Pro-Clp-Gly;
(SEQ ID NO: 363)
Pro-Clp-Gly-Pro-Lys-Gly-Pro-Clp-Gly-Pro-Clp-Gly-
Pro-Clp-Gly-Pro-Clp-Gly-Pro-Clp-Gly;
(SEQ ID NO: 364)
Pro-Clp-Gly-Pro-Clp-Gly-Lys-Clp-Gly-Pro-Clp-Gly-
Pro-Clp-Gly-Pro-Clp-Gly-Pro-Clp-Gly;
(SEQ ID NO: 365)
Pro-Clp-Gly-Pro-Clp-Gly-Pro-Lys-Gly-Pro-Clp-Gly-
Pro-Clp-Gly-Pro-Clp-Gly-Pro-Clp-Gly;
(SEQ ID NO: 366)
Pro-Clp-Gly-Pro-Clp-Gly-Pro-Clp-Gly-Lys-Clp-Gly-
Pro-Clp-Gly-Pro-Clp-Gly-Pro-Clp-Gly;
(SEQ ID NO: 367)
Pro-Clp-Gly-Pro-Clp-Gly-Pro-Clp-Gly-Pro-Lys-Gly-
Pro-Clp-Gly-Pro-Clp-Gly-Pro-Clp-Gly;
(SEQ ID NO: 368)
Pro-Clp-Gly-Pro-Clp-Gly-Pro-Clp-Gly-Pro-Clp-Gly-
Lys-Clp-Gly-Pro-Clp-Gly-Pro-Clp-Gly;
(SEQ ID NO: 369)
Pro-Clp-Gly-Pro-Clp-Gly-Pro-Clp-Gly-Pro-Clp-Gly-
Pro-Lys-Gly-Pro-Clp-Gly-Pro-Clp-Gly;
(SEQ ID NO: 370)
Pro-Clp-Gly-Pro-Clp-Gly-Pro-Clp-Gly-Pro-Clp-Gly-
Pro-Clp-Gly-Lys-Clp-Gly-Pro-Clp-Gly;
(SEQ ID NO: 371)
Pro-Clp-Gly-Pro-Clp-Gly-Pro-Clp-Gly-Pro-Clp-Gly-
Pro-Clp-Gly-Pro-Lys-Gly-Pro-Clp-Gly;
(SEQ ID NO: 372)
Pro-Clp-Gly-Pro-Clp-Gly-Pro-Clp-Gly-Pro-Clp-Gly-
Pro-Clp-Gly-Pro-Clp-Gly-Lys-Clp-Gly;
(SEQ ID NO: 373)
Pro-Clp-Gly-Pro-Clp-Gly-Pro-Clp-Gly-Pro-Clp-Gly-
Pro-Clp-Gly-Pro-Clp-Gly-Pro-Lys-Gly;
(SEQ ID NO: 374)
Lys-Hyp-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 375)
Clp-Lys-Gly-Clp-Hyp-Gly-Clp-Hyp-Gly-Clp-Hyp-Gly-
Clp-Hyp-Gly-Clp-Hyp-Gly-Clp-Hyp-Gly;
(SEQ ID NO: 376)
Clp-Hyp-Gly-Lys-Hyp-Gly-Clp-Hyp-Gly-Clp-Hyp-Gly-
Clp-Hyp-Gly-Clp-Hyp-Gly-Clp-Hyp-Gly;
(SEQ ID NO: 377)
Clp-Hyp-Gly-Clp-Lys-Gly-Clp-Hyp-Gly-Clp-Hyp-Gly-
Clp-Hyp-Gly-Clp-Hyp-Gly-Clp-Hyp-Gly;
(SEQ ID NO: 378)
Clp-Hyp-Gly-Clp-Hyp-Gly-Lys-Hyp-Gly-Clp-Hyp-Gly-
Clp-Hyp-Gly-Clp-Hyp-Gly-Clp-Hyp-Gly;
(SEQ ID NO: 379)
Clp-Hyp-Gly-Clp-Hyp-Gly-Clp-Lys-Gly-Clp-Hyp-Gly-
Clp-Hyp-Gly-Clp-Hyp-Gly-Clp-Hyp-Gly;
(SEQ ID NO: 380)
Clp-Hyp-Gly-Clp-Hyp-Gly-Clp-Hyp-Gly-Lys-Hyp-Gly-
Clp-Hyp-Gly-Clp-Hyp-Gly-Clp-Hyp-Gly;
(SEQ ID NO: 381)
Clp-Hyp-Gly-Clp-Hyp-Gly-Clp-Hyp-Gly-Clp-Lys-Gly-
Clp-Hyp-Gly-Clp-Hyp-Gly-Clp-Hyp-Gly;
(SEQ ID NO: 382)
Clp-Hyp-Gly-Clp-Hyp-Gly-Clp-Hyp-Gly-Clp-Hyp-Gly-
Lys-Hyp-Gly-Clp-Hyp-Gly-Clp-Hyp-Gly;
(SEQ ID NO: 383)
Clp-Hyp-Gly-Clp-Hyp-Gly-Clp-Hyp-Gly-Clp-Hyp-Gly-
Clp-Lys-Gly-Clp-Hyp-Gly-Clp-Hyp-Gly;
(SEQ ID NO: 384)
Clp-Hyp-Gly-Clp-Hyp-Gly-Clp-Hyp-Gly-Clp-Hyp-Gly-
Clp-Hyp-Gly-Lys-Hyp-Gly-Clp-Hyp-Gly;
(SEQ ID NO: 385)
Clp-Hyp-Gly-Clp-Hyp-Gly-Clp-Hyp-Gly-Clp-Hyp-Gly-
Clp-Hyp-Gly-Clp-Lys-Gly-Clp-Hyp-Gly;
(SEQ ID NO: 386)
Clp-Hyp-Gly-Clp-Hyp-Gly-Clp-Hyp-Gly-Clp-Hyp-Gly-
Clp-Hyp-Gly-Clp-Hyp-Gly-Lys-Hyp-Gly;
and
(SEQ ID NO: 387)
Clp-Hyp-Gly-Clp-Hyp-Gly-Clp-Hyp-Gly-Clp-Hyp-Gly-
Clp-Hyp-Gly-Clp-Hyp-Gly-Clp-Lys-Gly.

Another suitable CMP for use according to the invention is a CMP having or comprising the sequence Hyp-Flp-Gly-Hyp-Flp-Gly-Hyp-Flp-Gly-Hyp-Flp-Gly-Hyp-Flp-Gly-Hyp-Flp-Gly-Hyp-Flp-Gly (SEQ ID NO:388).

Other suitable CMPs for use according to the invention is a CMP having or comprising the sequence Gly₃-(Pro-Hyp-Gly)₆ (SEQ ID NO:397), Gly₃-(Pro-Flp-Gly)₆ (SEQ ID NO:398), Gly₃-(Pro-Hyp-Gly)₇ (SEQ ID NO:399), Gly₃-(Pro-Flp-Gly)₇ (SEQ ID NO:400), Gly₃-(Pro-Hyp-Gly)₈ (SEQ ID NO:401), Gly₃-(Pro-Flp-Gly)₈ (SEQ ID NO:402), Gly₃-(Pro-Hyp-Gly)₉ (SEQ ID NO:403), Gly₃-(Pro-Flp-Gly)₉ (SEQ ID NO:404), (Pro-Hyp-Gly)₆-Tyr (SEQ ID NO:405), (Pro-Flp-Gly)₆-Tyr (SEQ ID NO:406), (Pro-Hyp-Gly)₇-Tyr (SEQ ID NO:407), (Pro-Flp-Gly)₇-Tyr (SEQ ID NO:408), (Pro-Hyp-Gly)₈-Tyr (SEQ ID NO:409), (Pro-Flp-Gly)₈-Tyr (SEQ ID NO:410), Cys-(Pro-Hyp-Gly)₃ (SEQ ID NO:411), Cys-(Pro-Flp-Gly)₃ (SEQ ID NO:412), Cys-(Pro-Hyp-Gly)₅ (SEQ ID NO:413), Cys-(Pro-Flp-Gly)₅ (SEQ ID NO:414), Cys-(Pro-Hyp-Gly)₇ (SEQ ID NO:415), and Cys-(Pro-Flp-Gly)₇ (SEQ ID NO:416), and other analogous CMPs which may be suitable for use as agents for modification of collagen in vitro and in vivo for use in therapeutic and/or diagnostic methods (see, e.g., U.S. Pat. Nos. 8,283,414, 8,883,964 and 10,632,168, and US Patent Publ. No. US 2020/0353056 A1, the disclosures of all of which are incorporated herein by reference in their entireties).

In certain other embodiments of the invention, the collagen mimetic peptide comprises, consists essentially of or consists of an amino acid sequence that is or corresponds to a 21-mer comprising seven repeats of a three amino acid sequence in which Ala has been substituted for proline₂ in SEQ ID NO:1, yielding a sequence of seven repeats of proline-alanine-glycine ((Pro-Ala-Gly)₇), i.e., an amino acid sequence of: Pro-Ala-Gly-Pro-Ala-Gly-Pro-Ala-Gly-Pro-Ala-Gly-Pro-Ala-Gly-Pro-Ala-Gly-Pro-Ala-Gly (SEQ ID NO:418). In other embodiments, the collagen mimetic peptide comprises, consists essentially of or consists of an amino acid sequence that is or corresponds to a 21-mer comprising seven repeats of a three amino acid sequence in which either Hyp or Flp has been substituted for proline₁ in SEQ ID NO:418, yielding a sequence of seven repeats of proline-alanine-glycine ((Pro-Ala-Gly)₇), i.e., amino acid sequences of: Hyp-Ala-Gly-Hyp-Ala-Gly-Hyp-Ala-Gly-Hyp-Ala-Gly-Hyp-Ala-Gly-Hyp-Ala-Gly-Hyp-Ala-Gly (SEQ ID NO:419) and Flp-Ala-Gly-Flp-Ala-Gly-Flp-Ala-Gly-Flp-Ala-Gly-Flp-Ala-Gly-Flp-Ala-Gly-Flp-Ala-Gly (SEQ ID NO:420).

Other suitable CMPs for use according to the invention is a CMP having or comprising the sequence (Pro-Gly-Glu)₇ (SEQ ID NO:421), (Pro-Gly-Gln)₇ (SEQ ID NO:422), (Pro-Gly-Pro)₇ (SEQ ID NO:423), (Hyp-Gly-Glu)₇ (SEQ ID NO:424), (Hyp-Gly-Gln)₇ (SEQ ID NO:425), (Flp-Gly-Glu)₇ (SEQ ID NO:426), (Flp-Gly-Gln)₇ (SEQ ID NO:427), (Hyp-Gly-Pro)₇ (SEQ ID NO:428), (Hyp-Gly-Gln)₇ (SEQ ID NO:429), (Hyp-Gly-Glu)₇ (SEQ ID NO:430), (Pro-Gly-Asp)₇ (SEQ ID NO:431), (Pro-Gly-Asn)₇ (SEQ ID NO:432), (Lys-Gly-Gln)₇ (SEQ ID NO:433), (Lys-Gly-Glu)₇ (SEQ ID NO:434), (Hyp-Ala-Gly)₇ (SEQ ID NO:435), (Flp-Ala-Gly)₇ (SEQ ID NO:436), (Hyp-Gly-Glu)₇ (SEQ ID NO:437), (Hyp-Gly-Gln)₇ (SEQ ID NO:438), (Flp-Gly-Glu)₇ (SEQ ID NO:439), (Flp-Gly-Gln)₇ (SEQ ID NO:440), (Hyp-Gly-Asp)₇ (SEQ ID NO:441), (Hyp-Gly-Asn)₇ (SEQ ID NO:442), (Flp-Gly-Asp)₇ (SEQ ID NO:443), (Flp-Gly-Asn)₇ (SEQ ID NO:444), (Hyp-Gly-Pro)₇ (SEQ ID NO:445), (Flp-Gly-Pro)₇ (SEQ ID NO:446), (Pro-Gly-Hyp)₇ (SEQ ID NO:447), (Flp-Gly-Hyp)₇ (SEQ ID NO:448), (Hyp-Gly-Hyp)₇ (SEQ ID NO:449), (Hyp-Gly-Flp)₇ (SEQ ID NO:450), (Pro-Gly-Flp)₇ (SEQ ID NO:451), (Flp-Gly-Flp)₇ (SEQ ID NO:452), and other analogous CMPs comprising one or more additional or substituted amino acids including one or more cysteine residues, one or more methionine residues and/or one or more lysine residues, inserted or substituted into locations in the foregoing CMP sequences according to the locations set forth for cysteine in SEQ ID Nos:10-135, for methionine in SEQ ID Nos:136-251, and for lysine in SEQ ID Nos:262-387.

In certain other embodiments, any of the foregoing CMPs may optionally have one or more cysteine residues, one or more methionine residues, and/or one or more lysine residues, attached to either the N-terminus or the C-terminus, or to both termini, of the amino acid sequence. Non-limiting examples of such CMPs include:
SEQ ID NO:1 with a cysteine residue attached at the N-terminus, i.e., Cys-((Pro-Pro-Gly)₇) (SEQ ID NO:453), at the C-terminus, i.e., (Pro-Pro-Gly)₇-Cys (SEQ ID NO:454), or at both termini, i.e., Cys-((Pro-Pro-Gly)₇)-Cys (SEQ ID NO:455);
SEQ ID NO:6 with a cysteine residue attached at the N-terminus, i.e., Cys-((Flp-Hyp-Gly)₇) (SEQ ID NO:456), at the C-terminus, i.e., (Flp-Hyp-Gly)₇-Cys (SEQ ID NO:457), or at both termini, i.e., Cys-((Flp-Hyp-Gly)₇)-Cys (SEQ ID NO:458);
SEQ ID NO:1 with a methionine residue attached at the N-terminus, i.e., Met-((Pro-Pro-Gly)₇) (SEQ ID NO:459), at the C-terminus, i.e., (Pro-Pro-Gly)₇-Met (SEQ ID NO:460), or at both termini, i.e., Met-((Pro-Pro-Gly)₇)-Met (SEQ ID NO:461);
SEQ ID NO:6 with a methionine residue attached at the N-terminus, i.e., Met-((Flp-Hyp-Gly)₇) (SEQ ID NO:462), at the C-terminus, i.e., (Flp-Hyp-Gly)₇-Met (SEQ ID NO:463), or at both termini, i.e., Met-((Flp-Hyp-Gly)₇)-Met (SEQ ID NO:464);
SEQ ID NO:1 with a lysine residue attached at the N-terminus, i.e., Lys-((Pro-Pro-Gly)₇) (SEQ ID NO:465), at the C-terminus, i.e., (Pro-Pro-Gly)₇-Lys (SEQ ID NO:466), or at both termini, i.e., Lys-((Pro-Pro-Gly)₇)-Lys (SEQ ID NO:467);
SEQ ID NO:6 with a lysine residue attached at the N-terminus, i.e., Lys-((Flp-Hyp-Gly)₇) (SEQ ID NO:468), at the C-terminus, i.e., (Flp-Hyp-Gly)₇-Lys (SEQ ID NO:469), or at both termini, i.e., Lys-((Flp-Hyp-Gly)₇)-Lys (SEQ ID NO:470).

In other embodiments, the sequences set forth in SEQ ID Nos: 453-458 may have one or more methionine or lysine residues substituted in place of the one or more cysteine residues therein. In other embodiments, the sequences set forth in SEQ ID Nos: 459-464 may have one or more cysteine or lysine residues substituted in place of the one or more methionine residues therein. In other embodiments, the sequences set forth in SEQ ID Nos: 465-470 may have one or more methionine or cysteine residues substituted in place of the one or more lysine residues therein.

It will be understood by those of ordinary skill, of course, based on knowledge in the art and the teachings herein, that such CMPs may comprise two or more cysteine, methionine and/or lysine residues, in which at least one additional cysteine, methionine and/or lysine residue, or any combination thereof, may be substituted for at least one proline residue, at least one hydroxyproline residue, at least one fluoroproline residue and/or at least one chloroproline residue in any of the foregoing CMP sequences that comprise at least one proline, at least one hydroxyproline, at least one fluoroproline and/or at least one chloroproline residue. It also will be appreciated by those of ordinary skill in the art based on the teachings herein and information readily available in the art that other combinations of amino acid substitutions are also possible and within the scope of the present invention.

The CMPs described herein are suitable for a variety of purposes. For example, as described in further detail elsewhere herein, the CMPs may be used in a variety of therapeutic applications or preventative applications by being directly applied to or introduced into the body of a human or veterinary animal, particularly at sites of collagen disruption or potential collagen disruption, where the CMPs described herein will localize directly to the site of collagen disruption, anneal to disrupted collagen strands and stabilize the collagen structure such that it resists further disruption, and in some cases reform a native collagen triple helix in the site of collagen disruption. Such applications are useful in promoting the repair and strengthening of disrupted collagen in sites of injury or potential injury or disruption, for example in wounds, diseases, structural abnormalities or disorders (e.g., scarring, wrinkle formation, etc.) involving skin, tendon, ligament, cartilage, bone and other collagen-containing structures and organs. The CMPs described herein also are useful in providing biocompatible coatings for certain medical devices, to promote the healing of injuries and disorders in areas of the body where such devices are used in treating or preventing certain diseases, disorders and structural abnormalities or injuries in humans and veterinary animals, particularly those in which such diseases, disorders and structural abnormalities or injuries involve disruption of collagen and/or collagen-containing structures. The CMPs described herein also are useful in providing a unique delivery vehicle suitable for delivering a variety of therapeutic compounds, compositions and medicaments to sites of disease, disorder and structural abnormality or injury in humans and veterinary animals, particularly for use in treating, preventing or ameliorating diseases, disorders, medical conditions and structural abnormalities or injuries in which collagen disruption is either the cause of, is associated with, or is colocalized with the site of the disease, disorder and structural abnormality or injury. In additional embodiments, the CMPs described herein are useful in providing diagnostic agents suitable for diagnosing or detecting a disease, disorder, structural abnormality or injury in humans and veterinary animals. In certain such aspects, the CMPs may be either co-formulated with or conjugated directly or indirectly to one or more suitable diagnostic compounds, agents, labels and the like (see, e.g., U.S. Pat. Nos. 8,283,414, 8,883,964 and 10,632,168, and US Patent Publ. No. US 2020/0353056 A1, the disclosures of all of which are incorporated herein by reference in their entireties). Other suitable uses of the CMPs described herein and used in certain aspects of the present invention will be readily apparent to the ordinarily skilled artisan based on the disclosure herein and information that is readily available in the art.

In certain embodiments, the CMPs described herein are suitable for formation into a film, wafer, membrane or gel comprising one or more of the CMPs in a form suitable for introduction or implantation into a human or animal for therapeutic, preventative or diagnostic applications such as those described herein and others that will be familiar to those of ordinary skill in the relevant arts. For example, films, wafers, membranes, spheres, nanoparticles or gels can be formed from a solution of one or more of the CMPs described herein using methods such as those described in U.S. Pat. Nos. 6,197,934; 6,448,378; and 9,289,396; the disclosures of all of which are incorporated herein by reference in their entireties. Alternatively, films, wafers, membranes spheres, nanoparticles, or gels can be formed from other materials, such as atelocollagen (see U.S. Pat. Nos. 6,197,934; 6,448,378; and 9,289,396), copolymers of poly(lactic acid) and poly(glycolic acid) (PLGA) (see Bala, I., et al., Crit. Rev. Ther. Drug Carrier Syst. 21(5):387-422 (2004)), and other materials that are known to those of ordinary skill in the art (see, e.g., Kumar, V., et al., eds., “Polymer Gels: Perspectives and Applications”, ISBN 978-981-10-6079-3, Singapore: Springer (2018)), and one or more of the CMPs can be suitably incorporated into such films, wafers, membranes, spheres, nanoparticles, gels, etc., during the formation thereof by including the CMPs in the solution, at concentrations of about 1%-99%, about 2%-95%, about 3%-90%, about 4%-90%, about 5%-90%, about 10%-90%, about 15%-90%, about 20%-90%, about 25%-90%, about 25%-85%, about 25%-75%, about 25%-50%, about 35%-50%, and the like. Suitable other amounts or concentrations of the CMPs described herein that can be suitably included in the solutions during formation of the films, wafers, membranes, spheres, nanoparticles, gels, etc., will be readily apparent from the teachings herein and from information readily available in the art to the ordinarily skilled artisan. In certain such embodiments, one or more therapeutic compounds described herein, and/or one or more CMP-TC conjugates described herein, can be suitably incorporated into the solution from which the films, wafers, membranes, spheres, nanoparticles, gels, etc., are formed. Alternatively, in related aspects, one or more films, wafers, membranes, spheres, nanoparticles, gels, etc., once formed as described above, can be treated or coated with one or more CMPs and/or CMP-TC conjugates described herein, by immersing the films, wafers, membranes, spheres, nanoparticles, gels, etc., in a solution, particularly a buffered aqueous solution, containing a suitable amount or concentration (such as those described herein) of one or more CMPs or CMP-TC conjugates described herein, and then drying the films, wafers, membranes, etc., prior to use in therapeutic, preventative or diagnostic methods such as those described herein.

### Attachment/Conjugation of CMPs

In certain embodiments of the invention, the CMPs described herein are suitably attached or conjugated to one or more therapeutic or diagnostic compounds, to produce CMP conjugate compounds. In such embodiments of the invention, the CMP-therapeutic compound or CMP-diagnostic compound conjugate compounds can then be introduced into the body of a human or veterinary animal, in methods of treating and/or preventing and/or diagnosing certain diseases, disorders and structural abnormalities in humans or veterinary animals suffering therefrom. Accordingly, in certain embodiments the present invention also provides the use of the CMPs described herein attached or conjugated to one or more therapeutic compounds to produce conjugated CMPs, compositions comprising such conjugated CMPs (which may optionally comprise one or more additional therapeutic or pharmaceutically active ingredients), methods of producing such conjugates and methods of using such conjugates and compositions in the treatment, prevention and diagnosis of a variety of diseases, disorders and medical conditions in humans and veterinary animals.

Conjugates of CMPs and at least one therapeutic compound (which may be described herein as “CMP-TC conjugates”) according to this aspect of the invention will comprise at least one CMP described herein attached to at least one therapeutic compound to form a CMP-TC conjugate. CMPs suitably used in such aspects of the invention include any of those described herein, including CMPs having an amino acid sequence corresponding to any one of SEQ ID NOs:1-387 and particularly wherein the CMPs have an amino acid sequence corresponding to any one of SEQ ID NOs:1-14, 66-94, 107-135, 136-140, 192-220, 233-261, 260-264, 280, 281, 293, 294, 306, 307, 318-346, 347, 348, 359-388, and 397-416, and more particularly CMPs having amino acid sequences corresponding to SEQ ID NOs:10-27, 81-94, 122-135, 207-220, 248-261, 333-346, 374-388 and 397-416. Other suitable CMP sequences will be immediately apparent to one of ordinary skill in the art based on the teachings contained herein. For example, a CMP having at least one, and in some cases more than one, cysteine, methionine or lysine residue substituted in place of at least one, and in some cases more than one, proline, hydroxyproline, fluoroproline or chloroproline residue in SEQ ID NOs:1-9, will be particularly suitable for use in producing the CMP-TC conjugates provided by and used in the present invention. Examples of such suitable CMPs include those having amino acid sequences corresponding to SEQ ID NOs: 10-27, 81-94, 122-135, 207-220, 248-261, 333-346, 374-388, 397-416 and 453-470.

Methods of preparing the CMPs and CMP-TCs described herein and provided and used in the present invention will be familiar to those of ordinary skill in the art based on the teachings herein and information that is readily available in the art. For example, CMPs can be synthesized using standard protein/peptide synthesis techniques such as those described in U.S. Pat. Nos. 5,973,112; 7,122,521; and 7,858,741; as well as in U.S. Patent Publ. No. US 2007/0275897 A1, the disclosures of all of which are incorporated herein by reference in their entireties. Synthesis of CMPs can also be accomplished by purchasing custom-synthesized CMPs produced commercially, for example by Bachem (Torrance, CA, USA) and RS Synthesis (Louisville, KY, USA). In other embodiments, synthesis of CMPs can be accomplished using genetic engineering and recombinant expression of the CMPs from prokaryotic or eukaryotic expression systems (see, e.g., Buechter, D. D., et al., J. Biol. Chem. 278(1):645-650 (2003)).

In synthesizing the peptides described herein, in certain embodiments it is preferred that certain stereochemistries be used for the amino acid substitutions, particularly if hydroxyproline, fluoroproline or chloroproline are used:
(1) if hydroxyproline is substituted in place of proline in the Xaa position of the Xaa-Yaa-Gly trimer noted hereinabove, in certain embodiments the hydroxyproline has a (2R,4S) stereochemistry, or a cis or trans, and preferably a cis, stereochemistry;
(2) if hydroxyproline is substituted in place of proline in the Yaa position of the Xaa-Yaa-Gly trimer noted hereinabove, in certain embodiments the hydroxyproline has a (2R,4S) stereochemistry, or a cis or trans, and preferably a cis, stereochemistry;
(3) if fluoroproline is substituted in place of proline in the Yaa position of the Xaa-Yaa-Gly trimer noted hereinabove, in certain embodiments the hydroxyproline has a (2R,4S) stereochemistry, or a cis or trans, and preferably a cis, stereochemistry; and
(4) if chloroproline is substituted in place of proline in the Yaa position of the Xaa-Yaa-Gly trimer noted hereinabove, in certain embodiments the hydroxyproline has a (2R,4S) stereochemistry, or a cis or trans, and preferably a cis, stereochemistry.

Other suitable stereochemistries can be determined empirically without having to resort to undue experimentation, and will be immediately apparent to those of ordinary skill in the art. As noted above, certain CMPs provided by and used in the present invention may contain one or more additional substitutions, for example one or more cysteine residues and/or one or more methionine residues, in place of one or more prolines in a given CMP multimer. Such substitutions are suitably accomplished by adding those residues to the growing CMP peptide chain during the synthetic process using standard peptide synthetic methods such as those described elsewhere herein and those that are known in the art.

Once the CMPs have been prepared, they are suitably used in producing the CMP-TCs of the invention, i.e., the therapeutic or diagnostic compositions of the invention, by attaching one or more therapeutic compounds to the CMPs. In certain embodiments, the CMP-TCs of the invention can be prepared a method comprising (a) providing a collagen mimetic peptide having an amino acid sequence corresponding to any one of SEQ ID NOs:1-470, particularly CMPs have an amino acid sequence corresponding to any one of SEQ ID NOs:1-14, 66-94, 107-135, 136-140, 192-220, 233-261, 260-264, 280, 281, 293, 294, 306, 307, 318-346, 347, 348, 359-388, 397-416 and 453-470, and more particularly CMPs having amino acid sequences corresponding to SEQ ID NOs:10-27, 81-94, 122-135, 207-220, 248-261, 333-346, 374-388, 397-416 and 453-470; (b) providing at least one therapeutic or diagnostic compound suitable to be conjugated to the CMP; and (c) attaching the therapeutic or diagnostic compound directly or indirectly to the CMP. In certain cases, particularly wherein the therapeutic compound is a small peptide biologic compound, the therapeutic compound can be directly attached to the CMP via a peptide bond, for example by simply extending the synthesis of the peptide beyond the carboxy terminus of the CMP and attaching the amino terminal amino acid of the therapeutic compound to the carboxy terminal amino acid of the CMP via a peptide bond. One example of such a CMP-TC is a peptide conjugate in which the wound healing peptide known as Substance P and having an amino acid sequence of Arg-Pro-Lys-Pro-Gln-Gln-Phe-Phe-Gly-Leu-Met (SEQ ID NO:471), is attached to a CMP described herein. Examples of such conjugates include, for example:

(SEQ ID NO: 472)
Pro-Pro-Gly-Pro-Pro-Gly-Pro-Pro-Gly-Pro-Pro-
Gly-Pro-Pro-Gly-Pro-Pro-Gly-Pro-Pro-Gly-Arg-
Pro-Lys-Pro-Gln-Gln-Phe-Phe-Gly-Leu-Met;
(SEQ ID NO: 473)
Hyp-Pro-Gly-Hyp-Pro-Gly-Hyp-Pro-Gly-Hyp-Pro-
Gly-Hyp-Pro-Gly-Hyp-Pro-Gly-Hyp-Pro-Gly-Arg-
Pro-Lys-Pro-Gln-Gln-Phe-Phe-Gly-Leu-Met;
(SEQ ID NO: 474)
Pro-Hyp-Gly-Pro-Hyp-Gly-Pro-Hyp-Gly-Pro-Hyp-
Gly-Pro-Hyp-Gly-Pro-Hyp-Gly-Pro-Hyp-Gly-Arg-
Pro-Lys-Pro-Gln-Gln-Phe-Phe-Gly-Leu-Met;
(SEQ ID NO: 475)
Flp-Hyp-Gly-Flp-Hyp-Gly-Flp-Hyp-Gly-Flp-Hyp-
Gly-Flp-Hyp-Gly-Flp-Hyp-Gly-Flp-Hyp-Gly-Arg-
Pro-Lys-Pro-Gln-Gln-Phe-Phe-Gly-Leu-Met;
(SEQ ID NO: 476)
Clp-Hyp-Gly-Clp-Hyp-Gly-Clp-Hyp-Gly-Clp-Hyp-
Gly-Clp-Hyp-Gly-Clp-Hyp-Gly-Clp-Hyp-Gly-Arg-
Pro-Lys-Pro-Gln-Gln-Phe-Phe-Gly-Leu-Met;
(SEQ ID NO: 477)
Pro-Flp-Gly-Pro-Flp-Gly-Pro-Flp-Gly-Pro-Flp-
Gly-Pro-Flp-Gly-Pro-Flp-Gly-Pro-Flp-Gly-Arg-
Pro-Lys-Pro-Gln-Gln-Phe-Phe-Gly-Leu-Met;
and
(SEQ ID NO: 478)
Pro-Clp-Gly-Pro-Clp-Gly-Pro-Clp-Gly-Pro-Clp-
Gly-Pro-Clp-Gly-Pro-Clp-Gly-Pro-Clp-Gly-Arg-
Pro-Lys-Pro-Gln-Gln-Phe-Phe-Gly-Leu-Met.

In other methods of the invention, the one or more therapeutic or diagnostic compounds are suitably conjugated or attached to the CMPs via a covalent bond other than a peptide bond (see, e.g., U.S. Pat. Nos. 3,283,414 and 3,883,964, which are incorporated herein by reference in their entireties). For example, therapeutic compounds can be attached directly to a cysteine or methionine residue on a CMP described herein by covalently bonding a hydroxyl or amino group on an amino acid residue (e.g., a lysine residue) on the therapeutic or diagnostic compound (if it is a biologic molecule) to a sulfhydryl group on the cysteine or methionine residue of the CMP. Alternatively, if the CMP does not contain a cysteine or methionine residue, the one or more therapeutic or diagnostic compounds can be attached or conjugated to the CMP by a reaction between a hydroxyl group or amino group on the CMP and a sulfhydryl group on an amino acid residue (e.g., at a cysteine or methionine residue) on the therapeutic or diagnostic compound (if it is a biologic molecule). In yet another alternative method of conjugation, therapeutic compounds can be attached directly to a lysine residue on a CMP described herein by covalently bonding the therapeutic compound to an amino group on the lysine, for example using NHS ester conjugation (see, e.g., Mattson, G., et al., Molec. Biol. Rep. 17:167-183 (1993); Grabarek, Z. and Gergely, J., Anal. Biochem. 185:131-135 (1990); Staros, J. V. et al., Anal. Biochem. 156:220-2 (1986); Timkovich, R., Anal. Biochem. 79:135-43 (1977)). Such direct covalent attachments or conjugations between the CMP and the therapeutic/diagnostic compound can be accomplished using standard reaction techniques that will be familiar to those of ordinary skill in organic chemistry.

In other embodiments, particularly those wherein the therapeutic or diagnostic compound is not a biologic (and therefore does not have a peptide structure or amino acid residues having groups suitably attachable to cysteine, methionine, lysine or other residues on the CMP), such as small molecule organic or inorganic therapeutic or diagnostic compounds, the at least one therapeutic or diagnostic compound is indirectly attached to the collagen mimetic peptide via use of an attachment means. In such embodiments, the attachment means has two attachable ends, one of which attaches to an amino acid residue, and suitably a sulfhydryl group on a cysteine or methionine residue or an amino group on a lysine residue, of a CMP, and the other of which attaches to a hydroxyl or amino group on the therapeutic or diagnostic compound. For example, in certain such embodiments the attachment means comprises at least one polymeric chain having a first end and a second end, and the first end of the polymeric chain binds to the sulfhydryl group on a cysteine or methionine residue or an amino group on a lysine residue on the collagen mimetic peptide and the opposite or second end of the polymeric chain binds to an amino group or hydroxyl group on the therapeutic compound. In embodiments where the therapeutic or diagnostic compound is a biologic that is not suitable for direct attachment via peptide synthesis as described elsewhere herein, the second end of the attachment means can be attached to an amino group on an amino acid residue, such as a lysine residue, on the biologic therapeutic or diagnostic compound. Suitable such attachment means are well-known to those of ordinary skill in the art. For example, one attachment means suitable for use in accordance with this aspect of the invention includes a moiety which is a polymeric chain that on one end (the CMP-binding end in particular) comprises a sulfhydryl-binding group such as a maleimide, and on the other end (the therapeutic or diagnostic compounding-binding end in particular) comprises an amino-binding group such as N-hydroxysuccinimide. In certain such embodiments, the polymeric chain is a linear polyethyleneglycol chain comprising at least four ethyleneglycol monomers, e.g., from four to fifty ethyleneglycol monomers, from ten to forty ethyleneglycol monomers, from fifteen to thirty ethyleneglycol monomers, from fifteen to twenty-five ethyleneglycol monomers, from twenty to twenty-five ethyleneglycol monomers, and particularly four, six, eight, twelve, twenty, twenty-two, twenty-three, twenty-four or twenty-five ethyleneglycol monomers. Such attachment means suitable for attaching one or more therapeutic or diagnostic compounds to a CMP by the methods described herein are available commercially, e.g., from Thermo Fisher Scientific (Waltham, MA) (e.g., SM(PEG)6, SM(PEG)8, SM(PEG)12 and SM(PEG)24). By adjusting the length of the polymer chain, the bioavailability and sustainability of the therapeutic or diagnostic compound in vivo can be modulated—the use of longer polymer chains, e.g., a polymer comprising 24 ethyleneglycol monomers, will increase the rate of bioavailability of the compound once the CMP-TC has been introduced into the body of the human or veterinary animal, while the use of shorter polymer chains, e.g., a polymer comprising six ethyleneglycol monomers, will decrease the rate of bioavailability and thus increase the sustainability (or, in other words, will result in delayed release or sustained release) of the therapeutic or diagnostic compound. Other conjugates using linear or star-shaped PEG moieties which may be suitably prepared using the CMPs of the present invention, and used in the therapeutic and diagnostic methods of the invention, are disclosed in U.S. Pat. Nos. 8,283,414 and 8,883,964, which are incorporated herein by reference in their entireties. Hence, according to certain such aspects of the invention, the at least one therapeutic compound comprises at least one reactive hydroxyl group capable of being cross-linked to the collagen mimetic peptide using a polymeric linker.

Other indirect attachment methods for conjugating the one or more therapeutic or diagnostic compounds into or onto the CMPs also are suitably used according to the invention. For example, the at least one therapeutic or diagnostic compound can be enclosed within at least one nanoparticle that is attached via an attachment means, such as those described herein, to the collagen mimetic peptide. Alternatively, the collagen mimetic peptide can suitably comprise at least one biotin moiety and the therapeutic molecule can suitably comprise at least one avidin or streptavidin moiety, and the biotin moiety on the collagen mimetic peptide will bind to the avidin or streptavidin moiety on the therapeutic or diagnostic compound, thereby attaching the collagen mimetic peptide to the therapeutic or diagnostic compound. Of course, the alternative is also suitable for use, in which the collagen mimetic peptide can suitably comprise at least one avidin or streptavidin moiety and the therapeutic or diagnostic compound can suitably comprise at least one biotin moiety, and the biotin moiety on the at least one therapeutic or diagnostic compound will bind to the avidin or streptavidin moiety on the collagen mimetic peptide, thereby attaching the collagen mimetic peptide to the therapeutic compound.

Thus, according to certain embodiments of the invention, the therapeutic or diagnostic compounds can be suitably attached directly to the CMPs described herein. In other embodiments of the invention, the one or more therapeutic or diagnostic compounds can be attached indirectly to the CMPs described herein, for example via the use of a spacer, linker or bridge moiety. It is to be understood that whether the one or more therapeutic compounds are attached directly or indirectly to the CMPs, such attachment results in the production of conjugates of the CMPs and the one or more therapeutic compounds, which may be defined herein as CMP-TC conjugates.

Suitable therapeutic or diagnostic compounds for attachment or conjugation to the CMPs to produce the CMP-TCs of the present invention include any compound that has been shown to have particular therapeutic or preventative properties against one or more diseases, disorders, physical conditions or afflictions when introduced into a human or veterinary animal suffering from or predisposed to such diseases, disorders, physical conditions or afflictions. Provided that the therapeutic or diagnostic compound is capable of being conjugated or attached to at least one CMP according to the teachings herein, any therapeutic or diagnostic compound can be used in the conjugates, compositions and methods of the present invention. Suitable such therapeutic compounds may be biologic or non-biologic (e.g., so-called “small molecule”) therapeutic compounds. Compounds suitable for use include, but are not limited to, a steroidal anti-inflammatory drug, (e.g., prednisolone or a pharmaceutically acceptable salt thereof, such as prednisolone acetate), a nonsteroidal anti-inflammatory drug (e.g., acetylsalicylic acid, acetaminophen, ibuprofen, naproxen, nepafenac, bromfenac, diclofenac, flurbiprofen, ketoprofen, ketorolac, and an indene derivative (e.g., indomethacin, sulindac (Clinoril) and the like; see, e.g., U.S. Pat. No. 7,601,874, which is incorporated herein by reference in its entirety, for other indene derivatives suitably used as active pharmaceutical ingredients), and pharmaceutically acceptable salts, esters and derivatives thereof), a topical anesthetic (e.g., tetracaine, lidocaine, oxybuprocaine, proparacaine, and the like), a vitamin or a vitamin derivative or vitamin precursor (e.g., retinol, tretinoin, retinal, carotene and other retinoids and retinoid derivatives or precursors; folate; α-tocopherol; calciferol; phylloquinone, menadione and other vitamin K forms, precursors or derivatives, ascorbate; and the like), a therapeutic enzyme or a therapeutic fragment thereof (e.g., a collagenase and a serine protease, or a therapeutically effective fragment thereof), an antibiotic (e.g., an aminoglycoside antibiotic (such as gentamycin, tobramycin, paromomycin, kanamycin, neomycin and amikacin, and a pharmaceutically acceptable salt or ester thereof, e.g., tobramycin sulfate), a fluoroquinolone antibiotic (such as moxifloxacin, gatifloxacin, levofloxacin, gemifloxacin, ciprofloxacin, norfloxacin and ofloxacin, and a pharmaceutically acceptable salt, ester or derivative thereof, e.g., moxifloxacin hydrochloride, ciprofloxacin hydrochloride and gatifloxacin hydrochloride), a sulfonamide antibiotic (such as sulfacetamide, sulfadiazine, sulfadimidine, sulfafurazole (sulfisoxazole), sulfisomidine (sulfaisodimidine), sulfadoxine, sulfamethoxazole, sulfamoxole, sulfanitran, sulfadimethoxine, sulfamethoxypyridazine, sulfametoxydiazine, sulfametopyrazine and terephtyl, and a pharmaceutically acceptable salt, ester or derivative thereof), a β-lactam antibiotic (such as a penicillin or a derivative thereof (for example penicillin G, penicillin V, a benzylpenicillin and phenoxymethylpenicillin), dicloxacillin, flucloxacillin, oxacillin, nafcillin, amoxicillin, an ampicillin, ticarcillin, piperacillin, ritipenem, a carbapenem (e.g., ertapenem, doripenem, imipenem and meropenem, and a pharmaceutically acceptable salt, ester or derivative thereof), a cephem (such as cefazolin, cefalexin, cefadroxil, cefapirin, cefaclor, cefotetan, cephamycin (cefoxitin), cefprozil, cefuroxime axetil, ceftriaxone, ceftazidime, cefoperazone, cefdinir, cefcapene, cefdaloxime, ceftizoxime, cefmenoxime, cefotaxime, cefpiramide, cefpodoxime, ceftibuten, cefditoren, cefepime, ceftaroline fosamil, ceftolozane, ceftobiprole, ceftiofur, cefquinome and cefovecin, and a pharmaceutically acceptable salt, ester or derivative thereof), a monobactam (such as aztreonam or a pharmaceutically acceptable salt, ester or derivative thereof) and a β-lactamase inhibitor (such as sulbactam, tazobactam, clavulanic acid and avibactam, and a pharmaceutically acceptable sat, ester or derivative thereof)) or a cyclic peptide antibiotic (such as cyclosporine), a therapeutic monoclonal antibody or a therapeutic fragment thereof (such as adalimumab, altumomab, atezolizumab, atlizumab, bevacizumab, canakinumab, catumaxomab, certolizumab, cetuximab, clivatuzumab, edrecolomab, efalizumab, fontolizumab, girentuximab, golimumab, infliximab, labetuzumab, MABp1 (Xilonix™) natalizumab, nimotuzumab, nivolumab, oregovomab, panitumumab, pembrolizumab, pemtumomab, pertuzumab, ramucirumab, ranibizumab, rituximab, ruplizumab, tracatuzumab, tocilizumab, trastuzumab, ustekinumab, vedolizumab, visilizumab, votumumab, zalutumumab and zanolimumab, and active fragments, combinations or conjugates thereof), a therapeutic fusion protein (in certain embodiments, a recombinant fusion protein such as aflibercept (Regeneron), etanercept (Amgen), alefacept (Astellas Pharma), abatacept (Bristol-Myers Squibb), rilonacept (Regeneron), romiplostim (Amgen) and belatacept (Bristol-Myers Squibb)), a prostaglandin analogue (such as latanoprost, travoprost, tafluprost, unoprostone, netarsudil, tatanoprostene bunod, netarsudil and bimatoprost, and pharmaceutically acceptable salts, esters and derivatives thereof), a growth factor (such as EGF, PDGF, TGF-β, IGF-1, VEGF, FGF-β, IGF-1) or a therapeutic or growth-promoting (particularly skin growth-promoting) fragment thereof, a neuropeptide (such as Substance P (SEQ ID NO: 471), an α-adrenergic antagonist (such as brimonidine, clonidine and apraclonidine, and pharmaceutically acceptable salts, esters or derivatives thereof), a β-adrenergic antagonist (such as timolol, propranolol, atenolol, levobunolol, carteolol, betaxolol, and pharmaceutically acceptable salts, esters and derivatives thereof, e.g., timolol maleate), a cell surface receptor antagonist (such as lifitegrast or etanercept), a carbonic anhydrase inhibitor (such as dorzolamide, brinzolamide, methazolamide and acetazolamide, and pharmaceutically acceptable salts, esters and derivatives thereof, e.g., dorzolamide hydrochloride), and pharmaceutically acceptable salts, esters and derivatives thereof. With certain such therapeutic compounds, administration simultaneously with the CMPs described herein, whether as a CMP-TC conjugate or simply with one or more CMPs and one or more TCs in an admixture or applied separately, may prevent, attenuate or lessen one or more adverse side effects of the therapeutic compound. For example, it is known that the therapeutic administration of certain fluoroquinolone antibiotics may cause damage to collagen and collagen-containing structures (e.g., tendons) in humans or veterinary animals who have been treated with fluoroquinolones (see, e.g., “FDA Drug Safety Communication: FDA updates warnings for oral and injectable fluoroquinolone antibiotics due to disabling side effects,” accessed Nov. 6, 2017, at https://www.fda.gov/Drugs/DrugSafety/ucm511530.htm). As a result, simultaneous or co-administration of one or more of the CMPs described herein with one or more fluoroquinolone antibiotics to a human or veterinary animal in need of treatment with fluoroquinolones may allow the patient to receive the therapeutic benefits of the fluoroquinolone while mitigating, ameliorating or avoiding the collagen disruption resulting from such therapy, as the CMP can localize to and repair areas of damaged collagen in vivo.

Other suitable therapeutic compounds for use in the CMP-TC compounds, compositions and conjugates of the present invention include other non-biologic small molecule therapeutic compounds, including but not limited to alkylating agents, anti-tumor antibiotics, antimetabolites, hormonal agents, plant alkaloids, angiogenesis inhibitor, GnRH agonists, tyrosine kinase inhibitors, and the like. Examples of such non-biologic small molecule therapeutic compounds suitably used in accordance with the invention include but are not limited to a nitrosourea, a lenalidomide, imatinib, penatrexed, bortexomib, abiraterone acetate, everolimus, taxol, docetaxel, paclitaxel, carbazitaxel, mitoxantrone, carboplatin, cisplatin, gemcitabine, doxorubicin, casodex, flutamide, enzalutamide, abiraterone, sipuleucel-T and ketoconazole. Other suitable non-biologic small molecule therapeutic compounds that are advantageously used in forming the CMP-TC conjugates of the present invention, particularly for producing CMP-TC conjugates that are useful in treating certain cancers and preventing tumor metastasis, include inhibitors of lysyl oxidase (LOX), lysyl oxidase-like 1 (LOXL1) and lysyl oxidase-like 2 (LOXL2) enzymes. Such inhibitors have been suggested to have potential therapeutic application in treating and/or preventing certain cancers and the metastasis of solid tumors (see, e.g., U.S. Pat. Nos. 5,201,456; 5,120,764; 5,252,608; 8,461,303; 8,658,167; 8,680,246; 9,176,139; 9,255,086; and 9,289,447; see also Erler, J. T., et al., Nature 440:1222-1226 (2006); Erler, J. T., et al., Cancer Cell 15(1):35-44 (2009); Bondareva, A., et al., PLoS ONE 4(5):e5620 (2009); Granchi, C., et al., ChemMedChem 4(10:1590-1594 (2009); and Fang, M., et al., Tumor Biol. 35:2871-2882 (2014); the disclosures of all of which are incorporated herein by reference in their entireties).

In related aspects of the invention, CMP-TC conjugates comprising one or more inhibitors of LOX or LOX-like enzymes are suitably used in treating and/or preventing certain fibrotic diseases and disorders that are mediated by oxidoreductase enzymes such as LOX and the LOX-like enzymes (e.g., LOXL1 and LOXL2) in humans and veterinary animals. Fibrotic diseases and disorders suitably treated and/or prevented according to this aspect of the invention include but are not limited to pulmonary fibrosis, liver cirrhosis, myocardial fibrosis, surgical scarring, systemic sclerosis, scleroderma, keloid formation, proliferative vitreo retinopathy, and other fibrotic diseases and disorders that will be familiar to those of ordinary skill in the relevant arts. Particularly useful inhibitors of LOX and the Lox-like proteins include β-aminopropionitrile and certain derivatives and prodrugs thereof (see, e.g., U.S. Pat. Nos. 5,201,456; 5,120,764; 5,252,608; 8,461,303; 8,680,246; 9,176,139; and 9,255,086; the disclosures of all of which are incorporated herein in their entireties), as well as antibodies (which may be polyclonal or, preferably monoclonal) and fragments or portions thereof which bind to and inhibit the activity or function of LOX and LOX-like enzymes (see, e.g., U.S. Pat. No. 8,461,303; the disclosure of which is incorporated herein in its entirety).

In additional embodiments, compounds or compositions can be prepared comprising one or more CMPs and one or more antigens, either in admixture or co-formulation of one or more CMPs with one or more antigens (and optionally with one or more pharmaceutically suitable carriers or excipients), or in other compounds or compositions in which the one or more antigens are linked or conjugated directly or indirectly to the one or more CMPs. According to certain such aspects, the antigen may be a complete antigen or antigenic determinant or a fragment thereof (e.g., a hapten) that is capable of inducing an immune response in a human or veterinary animal when presented in the appropriate physiological context to the immune system of the human or veterinary animal, such as in the form of administration of the compound, conjugate or composition in the form of a vaccine or immunization to the human or veterinary animal. Compounds, conjugates and compositions useful in such embodiments can be prepared via co-formulation or direct or indirect conjugation according to the methods described elsewhere herein for co-formulation and conjugation of therapeutic compounds with or to CMPs. Antigens or portions thereof suitable for use in such compounds, conjugates and compositions, and therefore in methods of use thereof, include any molecule or particle, or portion thereof, that is capable of inducing an immune response in the human or veterinary animal, including but not limited to antigens (e.g., proteins, toxins, lipids, and other antigenic moieties, molecules or complexes) arising from or produced by bacteria (in which the antigen may comprise the entire bacterium or a portion thereof, such as a cell wall or cell membrane component, a nuclear component or a toxin produced by the bacterium), viruses (in which the antigen may comprise the entire viral particle or a portion thereof, such as a coat component (e.g., a protein or lipid or portion thereof), a nuclear component, or an enzyme encoded by or which is a part of the viral particle), protists, fungi, plants (which may include plant irritants or allergens such as pollen particles), animals (from which the antigen or portion thereof may be an allogeneic antigen or autogeneic antigen, or a portion thereof), and the like; examples of such antigens or portions thereof will be readily familiar to those in the relevant arts. Such compounds, compositions or conjugates are suitably used in methods for treating and/or preventing one or more disorders, diseases and afflictions in humans and veterinary animals, for example through the use of the compounds, compositions or conjugates in creating an immune response in the animal or veterinary human. In certain such methods, a disease or disorder is treated and/or prevented in the animal or veterinary animal by administration of one or more of the compounds, compositions or conjugates of this aspect of the invention into the human or veterinary animal, such as in the form of a vaccine or immunization. Such vaccines or immunizations are suitably formulated according to methods that are well-known in the relevant arts, and are administered in any mode that will result in the development of an immune response by the human or veterinary animal to the antigen or portion thereof, thereby treating and/or preventing the disease or disorder caused directly or indirectly by the antigen or portion thereof. Such vaccines or immunizations can be administered to the human or veterinary animal by any suitable route, such as orally, parenterally (including subcutaneously, intradermally, transdermally, intrathecally or intravenously), via ocular administration (e.g., in the form of drops, gels, wafers, or via injection, as described elsewhere herein for CMP-TC administration to the eye), intranasally, and other routes of administration that will be familiar to those of ordinary skill. In such embodiments, the compounds, conjugates or compositions of the invention are suitably administered to the human or veterinary animal until an immune response is developed by the human or veterinary animal that is sufficient to treat and/or prevent the target disease or disorder, and may be readministered as necessary to boost the immune response and/or to ensure continued immunity to the target antigen or portion thereof. Diseases and disorders suitably treated by such methods of the invention include any disease or disorder involving or resulting from the activity of any foreign agent acting upon the cells, organs, organ systems, bodily structures or bodies of humans and veterinary animals, including but not limited to infectious diseases, cancers, allergies and other immune overreactions (e.g., graft-versus-host or host-versus-graft diseases), Stevens-Johnson Syndrome, mucus membrane pemphigoid, toxic epidermal necrolysis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, and the like.

Suitable diagnostic compounds for attachment or conjugation to CMPs to produce the conjugates and compositions of the invention include, but are not limited to, labeled probes, such as fluorescent dyes (e.g., quantum dots, indocyanine green, fluorescein, rhodamine, a merocyanine dye, a near-infrared fluorescent dye, and the like); a radioisotope, a nuclide used for PET, a nuclide used for SPECT, particularly wherein each of the radioisotope, the nuclide used for PET or SPECT is selected from the group consisting of ¹¹C, ¹³N, ¹⁵O, ¹⁸F, ⁶⁶Ga, ⁶⁷Ga, ⁶⁸Ga, ⁶⁰Cu, ⁶¹Cu, ⁶²Cu, ⁶⁷Cu, ⁶⁴Cu, ⁴⁸V, Tc-99m, ²⁴¹Am, ⁵⁵Co, ⁵⁷Co, ¹⁵³Gd, ¹¹¹In, ¹³³Ba, ⁸²Rb, ¹³⁹Ce, Te-123m, ¹³⁷Cs, ⁸⁶Y, ⁹⁰Y, ^{185/187}Re, ^{186/188}Re, ¹²⁵I, a complex thereof, and a combination thereof, and an MRI contrast medium, a CT contrast medium, and a magnetic material, particularly wherein each of the MRI contrast medium, the CT contrast medium, and the magnetic material is selected from the group consisting of gadolinium, Gd-DTPA, Gd-DTPA-BMA, Gd-HP-D03A, iodine, iron, iron oxide, chromium, manganese, a complex or chelate complex thereof, and a combination thereof. According to such aspects of the invention, the CMP and the labeled probe are suitably physically or chemically bound directly to each other, for example via a direct conjugation through a coordinate bond, a covalent bond, a hydrogen bond, a hydrophobic interaction or a physical adsorption, or indirectly via use of at least one attachment means such as those described herein and others that are known in the art. Methods of conjugating or attaching diagnostic compounds to proteins, such as CMPs, are known in the art (see, e.g., U.S. Publ. Patent Appl. No. US 2012/0195828 A1, the disclosure of which is incorporated herein in its entirety).

### Use of CMPs and CMP-TC Conjugates

Thus, the invention provides methods of preparing compositions that are useful in treating, preventing, diagnosing or ameliorating a disease, disorder or medical condition in humans or veterinary animals. In yet another aspect, the invention provides methods of treating, preventing, diagnosing or ameliorating a disease, disorder or medical or physical condition in humans or veterinary animals using the compositions of the invention. Particularly preferred CMPs for use in such aspects of the invention include CMPs comprising, consisting essentially of, or consisting of, CMPs having an amino acid sequence of (Pro-Pro-Gly)₇ (SEQ ID NO:1), (Flp-Pro-Gly)₇ (SEQ ID NO:4), (Pro-Flp-Gly)₇ (SEQ ID NO:5), (Flp-Hyp-Gly)₇ (SEQ ID NO:6), (Clp-Hyp-Gly)₇ (SEQ ID NO:9), (Hyp-Flp-Gly)₇ (SEQ ID NO:388), Gly₃-(Pro-Hyp-Gly)₆ (SEQ ID NO:397), Gly₃-(Pro-Flp-Gly)₆ (SEQ ID NO:398), Gly₃-(Pro-Hyp-Gly)₇ (SEQ ID NO:399), Gly₃-(Pro-Flp-Gly)₇ (SEQ ID NO:400), Gly₃-(Pro-Hyp-Gly)₈ (SEQ ID NO:401), Gly₃-(Pro-Flp-Gly)₈ (SEQ ID NO:402), Gly₃-(Pro-Hyp-Gly)₉ (SEQ ID NO:403), Gly₃-(Pro-Flp-Gly)₉ (SEQ ID NO:404), (Pro-Hyp-Gly)₆-Tyr (SEQ ID NO:405), (Pro-Flp-Gly)₆-Tyr (SEQ ID NO:406), (Pro-Hyp-Gly)₇-Tyr (SEQ ID NO:407), (Pro-Flp-Gly)₇-Tyr (SEQ ID NO:408), (Pro-Hyp-Gly)₈-Tyr (SEQ ID NO:409), (Pro-Flp-Gly)₈-Tyr (SEQ ID NO:410), Cys-(Pro-Hyp-Gly)₃ (SEQ ID NO:411), Cys-(Pro-Flp-Gly)₃ (SEQ ID NO:412), Cys-(Pro-Hyp-Gly)₅ (SEQ ID NO:413), Cys-(Pro-Flp-Gly)₅ (SEQ ID NO:414), Cys-(Pro-Hyp-Gly)₇ (SEQ ID NO:415), or Cys-(Pro-Flp-Gly)₇ (SEQ ID NO:416), and derivatives thereof comprising one or more cysteine, methionine or lysine residues such as those described elsewhere herein.

The CMPs and CMP-TC conjugates of the present invention, including solutions, gels, films, wafers, membranes, spheres, nanoparticles and suspensions comprising, consisting essentially of or consisting of the CMPs and/or CMP-TC conjugates of the present invention, are suitably used as or included in compositions for use in, or as, a medicament for treating, preventing or ameliorating a variety of diseases or disorders in humans or veterinary animals in need of treatment or prevention thereof. Other compositions provided by this aspect of the invention provide the use of CMPs conjugated to one or more diagnostic compounds or molecules, such as one or more labeled probes, which then are used as diagnostic reagents in a variety of tests and assays, particularly in vivo or in situ, to diagnose a disease, disorder, or physical condition in a human or veterinary animal. Such medicament compositions or diagnostic compositions may comprise, in addition to the CMPs, CMP-TC conjugates or CMPs conjugated to one or more diagnostic compounds or molecules, one or more additional therapeutic compounds or pharmaceutically active ingredients (e.g., one or more antibiotics, one or more growth factors, autologous plasma rich in growth factors (PRGF), one or more cytokines, one or more antibodies fragments thereof, one or more non-biologic small molecule therapeutic compounds, and pharmaceutically active salts, esters and derivatives thereof, and the like, including those described herein and others that are known in the art. The compositions of the invention may additionally or alternatively comprise one or more pharmaceutically acceptable carriers or excipients. Pharmaceutically acceptable carriers or excipients suitable for use in the compositions and methods of the invention include, for example, one or more solvents (which may include water, an organic solvent or an inorganic solvent), one or more buffers, one or more polymers, one or more salts, one or more sugars, one or more sugar alcohols, one or more disintegrating agents, one or more aerosolizing agents or carriers, one or more desiccants, and the like. Other pharmaceutically acceptable carriers or excipients suitable for use in the compositions of the present invention will be readily familiar to those of ordinary skill in the relevant arts.

Without wishing to be bound by theory, it is thought that the CMPs provided by the invention and used in the methods of the invention are useful in particular in repairing damaged collagen, particularly damaged helical collagen, that results from or that is involved in a variety of diseases, disorders, structural abnormalities, physical conditions and medical conditions in humans and veterinary animals. For example, when collagen is damaged structurally it is often hydrolyzed in one of the three helices forming triple helix collagen, thereby causing an unraveling and structural deformity of the triple helix. This disruption and digestion can also lead to collagen being fragmented and fractured into many smaller pieces which remain in the extracellular milieu or which find their way into the blood or lymphatic circulatory systems. Such fragments are ultimately either phagocytized or bound by scavenger cells, or bind to cell surface receptors on somatic cells in the human or veterinary animal. Such receptors (which may include, for example, integrins, discoidin-domain receptors, glycoprotein VI and leucocyte-associated immunoglobulin-like receptor-1 (LAIR-1)) control cellular functions such as growth, differentiation, morphogenesis, tissue repair, adhesion, migration, homeostasis, immune function and wound healing, are often disrupted, or their functions or signaling systems are up- or down-regulated, via the binding of such free collagen fragments. According to this theory, when CMPs encounter damaged collagen or fragments thereof they dynamically anneal to or bind the fractured collagen triple helix and structurally repair it, resulting in (among other things) the restoration of cellular receptors to their proper function and levels of signaling activity. Thus, in this way the aggregate result of the application of CMPs to a human or veterinary animal having a disease, disorder, structural abnormality or injury involving or resulting from damaged collagen is to unleash an accelerated wound healing process which in some physiological contexts includes rapid epithelial cell, endothelial cell or neural cell growth, migration and adhesion over the now repaired collagen matrix, resulting in the restoration of normal or near-normal structure and function of such cells, and tissues, organs and organ systems comprising such cells.

Diseases, disorders, physical conditions and medical conditions suitably treated, prevented, ameliorated or diagnosed using the compositions and methods of the invention include, but are not limited to ocular diseases or disorders, nerve or nervous system diseases or disorders and cardiovascular diseases or disorders. Suitable dosages of the compositions and conjugates of the invention for such uses are concentrations of the CMP component of the composition or conjugate at about 10 ng/ml to about 500 μg/ml, about 15 ng/ml to about 400 μg/ml, about 20 ng/ml to about 300 μg/ml, about 25 ng/ml to about 250 μg/ml, about 30 ng/ml to about 200 μg/ml, about 35 ng/ml to about 200 μg/ml, about 40 ng/ml to about 200 μg/ml, about 50 ng/ml to about 200 μg/ml, about 75 ng/ml to about 200 μg/ml, and about 100 ng/ml to about 200 μg/ml. In certain such embodiments, the conjugates or compositions are suitably applied to the tissue, organ or organ system being treated in dosages equivalent to a CMP concentration of about 25 μg/ml to about 500 μg/ml, e.g., about 25 μg/ml, about 30 μg/ml, about 35 μg/ml, about 40 μg/ml, about 45 μg/ml, about 50 μg/ml, about 75 μg/ml, about 100 μg/ml, about 125 μg/ml, about 150 μg/ml, about 175 μg/ml, about 200 μg/ml, about 225 μg/ml, about 250 μg/ml, about 300 μg/ml, about 350 μg/ml, about 400 μg/ml, about 450 μg/ml or about 500 g/ml. In particular such embodiments, concentrations equivalent to a CMP concentration of between about 25 μg/ml, 50 μg/ml, 75 μg/ml or 100 μg/ml are used. Suitable dosages of CMPs for human administration (per kilogram of body mass) are about 10 ng/Kg to about 1000 ng/Kg, about 15 ng/Kg to about 650 ng/Kg, about 20 ng/Kg to about 600 ng/Kg, about 25 ng/Kg to about 550 ng/Kg, about 30 ng/Kg to about 500 ng/Kg, about 20 ng/Kg to about 350 ng/Kg, and about 25 ng/Kg to about 300 ng/Kg, about 10 ng/Kg to about 250 ng/Kg, about 10 ng/Kg to about 200 ng/Kg, about 10 ng/Kg to about 150 ng/Kg, or about 10 ng/Kg to about 100 ng/Kg. Appropriate specific concentrations for administration within these ranges can also be determined by the clinician via titration while treating a human or veterinary animal for a specific cardiovascular, cerebrovascular, or neurological disease, disorder or condition, using methods well-known to practicing physicians and those of ordinary skill in the relevant pharmaceutical and medical arts. Additional concentrations, dosages and amounts of the conjugates or compositions of the invention that are suitably used in such methods can be easily determined by one of ordinary skill, based on the information contained herein and that is available in the art, without the need to resort to undue experimentation.

Ocular diseases or disorders that can be treated, prevented, ameliorated or diagnosed using the compositions and methods of the invention include but are not limited to those involving the cornea and/or the sclera of the eye, i.e., corneoscleral diseases, disorders and conditions. Without wishing to be bound by theory, it is thought that the compositions of the invention, particularly the one or more CMPs contained in such compositions, repair disordered, disorganized or digested collagen found in the extracellular matrix and/or stroma of the cornea and sclera in such a way as to repair the collagen matrix/stroma and restore the homeostatic structure and function (including stiffening or strengthening) of the cornea and/or sclera, thereby treating or ameliorating such diseases and disorders and the signs/symptoms thereof. In other aspects, the compositions of the invention, particularly the one or more CMPs contained in such compositions, prevent the disorganization and digestion of the collagen in the extracellular matrix and/or stroma of the cornea and sclera upon insult or injury, in such a way as to preserve the collagen matrix/stroma and thus the homeostatic structure and function of the cornea and/or sclera, thereby preventing such diseases and disorders and the signs/symptoms thereof. Examples of corneoscleral diseases, disorders and conditions that are suitably treated, prevented, ameliorated or diagnosed using the compositions and methods of the invention include but are not limited to myopia, presbyopia, hyperopia, keratitis, episcleritis, scleritis, corneal ulceration, sequelae of corneal ulceration, corneal ectasia, acquired abnormalities of corneal shape, keratoconus, corneal astigmatism, keratoglobus, posterior corneal depressions, keratectasia, keratocele, descemetocele, pellucid marginal degeneration, Terrien's marginal dystrophy, Mooren's ulcers, central corneal ulcers, marginal corneal ulcers, staph marginal ulceration, Salzman's nodular dystrophy, age-related peripheral corneal atrophy, geographic ulceration, disciform stromal keratitis, metaherpetic ulceration, keratomalacia, post penetrating keratoplasty, incisional wounds, anterior membrane dystrophies, stromal dystrophies, ocular mucous membrane pemphigoid, necrotizing scleritis, scleromalacia, coloboma, scleral buckle induced scleromalacia, congenital hereditary stromal dystrophy, congenital anterior staphyloma, sclerocornea, traumatic breaks in Descemet's membrane, corneal keloids, scleral ectasia, scleral staphyloma, deep scleritis, necrotizing scleritis, scleromalacia perforans, hyaline degeneration of the sclera, paralimbal scleromalacia, ocular graft vs host disease, and choroideremia. In particular, the compositions and methods of the present invention are suitable for treating, ameliorating, preventing and/or diagnosing myopia, presbyopia, and keratoconus. Other corneoscleral diseases, disorders and conditions that may be suitably prevented, treated, ameliorated or diagnosed using the compositions and methods of the present invention will be apparent to one of ordinary skill in the art based on information readily available in the literature.

Other ocular diseases or disorders that can be treated, prevented, ameliorated or diagnosed using the compositions and methods of the invention include anterior segment diseases and disorders including but not limited to glaucoma, cataracts, vitreous adhesions or floaters, macular degeneration, dry eye syndrome (also known as dry eye disease), corneal keratitis, non-infectious corneal ulceration, non-infectious corneal melting, infectious corneal ulceration, infectious corneal melting, conjunctivitis, Stevens-Johnson Syndrome, iritis, uveitis, vitritis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, ectasia, corneal lacerations, corneal erosion, corneal abrasions, acute or chronic corneal pain (particularly that resulting from damage or injury to the corneal nerves or denervation; see, e.g., Rosenthal, P. and Borsook, D., Br J Ophthalmol. 2016; 100(1):128-134; Theophanous, C., et al., Optom. Vis. Sci. 2015; 92(9):e233-240; Belmonte, C., et al., Ocul. Surf 2004; 2(4):248-253; Belmonte, C., et al., Exp. Eye Res. 2004; 78(3):513-525; Belmonte, C., et al., Curr. Ophthalmol Rep. 2015; 3(2):111-121); Baratta, R O et al., Front. Pharmacol. 12:70563 (Aug. 16, 2021), doi:10.3389/fphar.2021.705623; Baratta, R O et al., Surv. Ophthalmol. 67:60-67 (2022); Ribeiro, M et al., Int. J. Mol. Sci. 23:2911 (2022); McGrady, N R et al., Front. Pharmacol. 12:8764709 (Nov. 2, 2021), doi:10.3389/fphar.2021.764709), including but not limited to paraocular pain, extraocular pain and post-herpetic neuralgia, and post-operative afflictions of the eye resulting from eye surgery. Such post-operative afflictions of the eye resulting from eye surgery can be, for example, afflictions arising post-operatively from cataract surgery or glaucoma surgery, particularly wherein those afflictions result in or are a post-operative state of the eye requiring medication.

Additional ocular diseases or disorders that can be treated, prevented, ameliorated or diagnosed using the compositions and methods of the invention include but are not limited to posterior segment diseases and disorders, particularly those involving the retina, including but not limited to macular degeneration (wet, dry and age-related), retinitis pigmentosa, retinal tears or detachment, retinopathy (e.g., diabetic retinopathy), arterial or venous occlusion (e.g., BRAO (Branch Retinal Artery Occlusion), CRAO (Central Retinal Artery Occlusion), BRVO (Branch Retinal Vein Occlusion) and CRVO (Central Retinal Vein Occlusion), optic neuritis, optic neuropathy (including, for example, AION (Anterior Ischemic Optic Neuropathy), and traumatic optic neuropathy), optic atrophy (e.g., glaucomatous optic atrophy), one or more neuropathies impacting the eye or area around the eye, including paraocular diseases, disorders or conditions and extraocular diseases, disorders or conditions, such as cranial nerve palsies including but not limited to Cranial III Nerve Palsy, Cranial Nerve IV Palsy, Cranial Nerve V Palsy (e.g., trigeminal neuralgia and post-herpes zoster neuralgia), Cranial Nerve VI Palsy and Cranial Nerve VII Palsy (e.g., Bell's Palsy)), and the like, and other retinal and posterior segment related disorders and diseases involving the retinal epithelium, particularly the retinal pigment epithelium, retinal blood vessels and/or retinal, cranial or optic nerves. Additional ocular disorders that are advantageously treated, ameliorated and/or prevented using the compositions and methods of the present invention include but are not limited to refractive eye disorders including myopia, presbyopia and amblyopia. For example, myopia is known to be associated with axial elongation of the eye, which may in part involve collagen disruption in the ocular sclera (see, e.g., Guo, P. et al., Trans. Vis. Sci. Tech. 9(9):45 (2020); Zhao, F. et al., Am. J. Pathol. 188:1754-1767 (2018)), and which may be ameliorated by scleral strengthening by cross-linking or other manipulation of the scleral connective tissue (see, e.g., Backhouse, S. et al., Ann. Eye Sci. 3:5 (2018); Grytz, R. et al., Curr. Opin. Biomed. Eng. 15:40-50 (2020); Garcia, M. B. Et al., Invest. Ophthalmol. Vis. Sci. 58:1875-1886 (2017)).

According to this aspect of the invention, methods of treating or preventing an ocular disease, disorder or wound in a human or veterinary animal suffering from or predisposed to an ocular disease, disorder or wound, comprise administering the compositions described herein, particularly the CMPs or CMP-TC conjugates and/or compositions comprising such conjugates, to an eye of a human or veterinary animal. Without wishing to be bound by theory, the inventors surmise that in areas of eye disease or disorder there is sufficient disruption of type I collagen such that the CMP will target the site of the eye disease or disorder specifically and intercalate into the collagen structure, for example by intercalating into one or more damaged helices of helical collagen, thereby directly reforming a functioning collagen helix or matrix or, in cases where the CMP is conjugated to a therapeutic compound, delivering the therapeutic compound to the site where it must act to treat, prevent or ameliorate the eye disease or disorder. In certain such anterior segment ocular diseases or disorders, such as acute or chronic corneal pain (including, but not limited to paraocular pain, extraocular pain, and post-herpetic neuralgia), denervated corneas suffer from poor healing capability and as such a topical therapy which can impact neuroregeneration would be a welcomed therapy in this area. Pain, both acute as well as chronic, is mediated by damaged corneal nerves (see, e.g., Rosenthal, P. and Borsook, D., Br J Ophthalmol. 2016; 100(1):128-134; Theophanous, C., et al., Optom. Vis. Sci. 2015; 92(9):e233-240; Belmonte, C., et al., Ocul. Surf. 2004; 2(4):248-253; Belmonte, C., et al., Exp. Eye Res. 2004; 78(3):513-525; Belmonte, C., et al., Curr. Ophthalmol Rep. 2015; 3(2):111-121), and thus a therapeutic which could be beneficial to nerve health would be clinically valuable for such patients. Based on the findings described herein relating to the behavior of dorsal root ganglion cells when exposed to certain CMPs of the invention after damage to a collagen support layer (see Example 1 hereinbelow), it can be expected that any cranial nerve would behave in a similar way. Corneal nerves, as branches of the trigeminal nerve, will therefore benefit from a therapy which includes the administration of one or more of the CMPs or CMP-TC conjugates described herein topically to the cornea. With a repaired and regenerated nerve, corneal recovery and pain relief would then follow, resulting in the amelioration of the acute or chronic corneal pain.

The conjugates or compositions are suitably applied to the eye in a concentration, dosage or amount sufficient to treat or prevent the ocular disease, disorder or wound, such as at those concentrations, dosages and amounts described hereinabove, and the condition of the eye in said human or veterinary animal is then monitored over time for improvement in the disease state or physical condition. If necessary, the conjugate or composition of the invention is then periodically readministered to the eye, according to dosing and treatment schedules and protocols described herein and others that will be familiar to the ordinarily skilled artisan, until the ocular disease, disorder or wound is cured, prevented or ameliorated. In such embodiments, the conjugates or compositions of the invention for treatment of anterior segment diseases and disorders can be suitably administered to the eye to the surface of the eye, conjunctivally, or subconjunctivally, particularly by administering the conjugate or composition dropwise onto the surface of the eye or into the subconjunctival fornix. In other embodiments, the conjugates or compositions of the invention for treatment of anterior segment diseases and disorders can be suitably administered to the front of the eye, including the sclera and the vitreous, either dropwise (relying on the ability of the CMP-containing formulations to migrate across the surface epithelium) or via injection, e.g., via intravitreal injection, according to methods that are well-known to those of ordinary skill in the medical and pharmaceutical arts. In other embodiments involving treatment, prevention, cure or diagnosis of posterior segment diseases and disorders, the conjugates and compositions of the invention can be administered to the posterior segment, e.g., at or near the retina, via mechanical introduction such as via injection using a needle or other suitable apparatus, or by administration of the conjugate or composition to the surface of the eye in the form of drops, in which the conjugate or composition (or component thereof, e.g., a CMP or CMP-TC conjugate) is transported or migrates to the posterior segment of the eye (e.g., at or near the retina). Administration of the conjugates or compositions to the eye can be accomplished by any well-known means, including applying the conjugates or compositions to the eye in the form of one or more drops or aliquots of a solution, a gel or a suspension that contains the composition or conjugates; via injection; in the form of a solid material such as a wafer or film (such as those described herein) that is implanted into an eye structure; in the form of a mesh or patch; by attaching the conjugate or composition to, or enclosing it within, one or more gels, spheres or nanoparticles that are then delivered into an eye structure. Other suitable methods of applying the conjugates or compositions to the eye to accomplish the therapeutic and diagnostic methods of the invention will be readily apparent to the ordinarily skilled artisan.

Nerve or nervous system (including the central nervous system (“CNS”) and peripheral nervous system (“PNS”) diseases or disorders that can be treated, prevented, ameliorated, reversed or diagnosed using the compositions and methods of the invention include but are not limited to injuries to one or more nerves or nerve processes (including axons, dendrites and neurons or neuronal bodies, ganglia, nerve bundles and the like), a neurodegenerative disease, injury or disorder including those that may involve the vascular system of the brain (in many different physiological or disease contexts such as multiple sclerosis, amyotrophic lateral sclerosis, Parkinson's disease, Alzheimer's disease, Huntington's disease, a traumatic encephalopathy, a non-Alzheimer's dementia (including but not limited to Lewy body dementia), encephalitis, meningitis, and the like), disorders or injuries involving peripheral nerves (such as a peripheral neuropathy such as diabetic peripheral neuropathy, nutritional neuropathy and alcohol-induced neuropathy, or an injury in which one or more peripheral nerves is severed or crushed), or certain neuroocular diseases and disorders including those involving or affecting the corneal nerves, retinal nerves and optic nerve, including but not limited to glaucoma, macular degeneration (wet and/or dry, which may or may not be age-related), neurotrophic keratitis, retinopathies (which may include diabetic retinopathy, ischemic retinopathy, a proliferative retinopathy, geographic atrophy, and other genetic-based retinopathies and genetic retinal diseases or disorders known in the art), damage to or inflammation of one or more corneal nerves (which may arise via damage to or inflammation of the eye via external diseases or trauma/wounding, including a transection of, or crush injury or torsional injury to, a nerve or nerve process), corneal pain (which may be acute or chronic, and which may result from damage or injury to the corneal nerves or corneal denervation, e.g., paraocular pain, extraocular pain, and post-herpetic neuralgia), an encephalopathy (e.g., traumatic encephalopathy such as concussion, encephalitis, meningitis), and the like. In certain such embodiments, the compositions and methods of the invention can be used to induce nerve repair or regrowth (e.g., via neuroregeneration), particularly in the cranial nerves including but not limited to the optic nerve, the retinal nerves, the acoustic nerve or the spinal nerve. In other such embodiments, the compositions and methods of the invention can be used to protect certain nerves from degeneration, or from further or continued degeneration (i.e., provide a neuroprotective function), which may, for example, be useful in preventing, reducing or slowing the progression of degeneration of the peripheral nerves for the prevention and/or treatment of diabetic peripheral neuropathy, nutritional neuropathy and alcohol-induced peripheral neuropathy, as well as of the corneal nerves, optic nerve and/or the retinal nerves for the prevention and/or treatment of corneal pain (e.g., acute corneal pain or chronic corneal pain, including but not limited to paraocular pain, extraocular pain, and post-herpetic neuralgia), glaucoma, genetic retinal diseases or disorders and genetic-based retinopathies (e.g., diabetic retinopathy). Other beneficial uses of the compositions and methods of the invention in treating, preventing, ameliorating or diagnosing nerve and nervous system diseases or disorders will be familiar to the ordinarily skilled artisan based on the guidance provided herein in view of information readily available in the relevant arts.

According to this aspect of the invention, methods of treating or preventing a nerve or nervous system disease or disorder in a human or veterinary animal suffering from or predisposed to a nerve or nervous system disease or disorder comprise administering the compositions described herein, particularly the CMPs and CMP-TC conjugates and compositions comprising such CMPs and/or conjugates, into or near one or more tissues, organs or organ systems of a human or veterinary animal, at a site proximal to the location of a nerve or nervous system lesion associated with or causing the nerve or nervous system disease or disorder. Without wishing to be bound by theory, the inventors surmise that in areas of certain nerve or nervous system diseases and disorders there is sufficient disruption of type I collagen (perhaps among other components of the local extracellular matrix) such that the CMP will target the site of the nerve or nervous system disease or disorder specifically and intercalate into the collagen structure, thereby inducing neuroregeneration and/or neuroprotection directly via reformation of a functioning collagen matrix, or in cases where the CMP carries a therapeutic compound delivering the therapeutic compound to the site where it must act to treat, prevent or ameliorate the nerve or nervous system disease or disorder. According to this aspect of the invention, the conjugates or compositions are suitably applied to, near or into the tissue, organ or organ system in a dosage, concentration and amount sufficient to treat, prevent or ameliorate the nerve or nervous system disease or disorder, such as those described hereinabove, and the progression, remission or stasis of the nerve or nervous system disease or disorder in the human or veterinary animal is then monitored over time for improvement in the disease or disorder state. Additional concentrations and amounts of the conjugates or compositions of the invention that are suitably used in such methods can be easily determined by one of ordinary skill, based on the information contained herein and that is available in the art, without the need to resort to undue experimentation. If necessary, the conjugate or composition of the invention is then periodically readministered into, near or onto one or more tissues, organs or organ systems according to dosing and treatment schedules and protocols described herein and that will be familiar to the ordinarily skilled artisan, until the nerve or nervous system disease or disorder is cured, prevented or ameliorated. In such embodiments, the conjugates or compositions of the invention are suitably administered to, near, on or into the tissues, organs or organ systems parenterally or topically. Parenteral administration is accomplished by any art-known route of administration of a therapy to the tissues, organ or organ systems, for example via a route selected from the group consisting of subcutaneous injection, intradermal injection, intramuscular injection, intracranial injection, intraspinal injection, or injection into any tissue, organ or organ system where a nerve or nervous system disease or disorder is being manifested; intravenous infusion; intraarterial infusion; endoscopic application; transdermal diffusion; implantation of a drug eluting wafer, film, gel or putty; sublingually; orally; or rectally. In certain such methods, the composition is suitably administered parenterally to the human or veterinary animal in the form of an injected solution or paste, a pill, a capsule, a solution, a suspension or a powder that is inhaled or ingested by the human or veterinary animal, or in the form of a mesh, film, wafer, gel, sphere, nanoparticle, paste, putty or patch that is implanted near, on or into the tissue, organ or organ system at or proximal to the site of the nerve or nervous system disease or disorder. In certain such embodiments, one or more of the compounds, compositions or conjugates of the invention may be coated onto or into a mesh or “sleeve” material such that the mesh or sleeve material is impregnated with one or more of the compounds, compositions or compositions of the invention, and the mesh or sleeve then applied to an injured (e.g., transected) or damaged nerve, nerve process or nerve bundle. In certain such aspects of the invention, the compositions and methods of the invention are used to improve the integrity of, and reverse the degradation of, the nerves and blood vessels associated with the PNS and CNS, including the cerebrovasculature in the brain, thereby treating, preventing, and/or reversing certain CNS and PNS diseases and disorders such as those described herein.

In other embodiments, administration of the conjugates or compositions to, near or into the tissues, organs or organ systems can be accomplished by any well-known means, including in the form of a solution, an ointment, a salve, a patch, a film, a gel, a paste, spheres, nanoparticles, putty, a cream, a topical solution and a drug eluting wafer. For example, the conjugates or compositions can be applied to or near, or introduced into, the tissues, organs or organ systems in the form of one or more drops of solution or a suspension that contains the composition or conjugates (for example, for use in the back of the eye, in the form of a topical transocular eyedrop); via injection; in the form of a coating on a solid material that is implanted into, near or onto the tissues, organs or organ systems; in the form of a mesh or patch; by attaching the conjugate or composition to, or enclosing it within, one or more nanoparticles that are then delivered into, near or on the tissues, organs or organ systems. Other suitable methods of applying the conjugates or compositions to, on, near or into the tissues, organs or organ systems to accomplish the therapeutic and diagnostic methods of the invention will be readily apparent to the ordinarily skilled artisan.

Cardiovascular diseases or disorders that can be treated, prevented, ameliorated or diagnosed using the compositions and methods of the invention include but are not limited to myocardial infarction, cardiac insufficiency, cardiac valve disorders, atherosclerosis (including but not limited to diabetic atherosclerosis), cardiomyophathy, arrhythmias, congenital heart disease, coronary artery disease, pericardial disease, vascular occlusive disease (e.g., affecting the carotid artery, the aorta, the vasculature of the brain (particularly the cerebrovasculature), the renal artery, the femoral artery, the pulmonary artery, and other large vessels and small vessels which may be arteries, arterioles, veins, venules and the like), cerebrovascular pathologies (including diabetic cerebrovascular diseases and disorders or traumatic injuries such as diseases and disorders characterized by blockage, trauma or leakage of the brain vasculature such as stroke, aneurysm, brain hemorrhages, brain microbleeds, chronic traumatic encephalopathy, and the like), bleeding disorders (e.g., arterial, venous or capillary leakage, including microbleeds in the tissues and organs such as the brain, skin, heart and other tissues, organs and organ systems), Marfan syndrome, and the like.

According to this aspect of the invention, methods of treating or preventing a cardiovascular disease or disorder in a human or veterinary animal suffering from or predisposed to a cardiovascular disease or disorder comprise administering the compositions described herein, particularly the CMPs and/or CMP-TC conjugates and compositions comprising such CMPs and/or conjugates, into the vascular system of a human or veterinary animal suffering from or predisposed to such a disease or disorder. Without wishing to be bound by theory, the inventors surmise that in areas of certain cardiovascular diseases and disorders there is sufficient disruption of type I collagen such that the CMP introduced into the vascular system of the subject will target the site of the cardiovascular disease or disorder specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the cardiovascular disease or disorder. According to this aspect of the invention, the conjugates or compositions are suitably applied to or into the vascular system in a dosage, concentration and amount sufficient to treat, prevent or ameliorate the cardiovascular system disease or disorder, such as those dosages, concentrations and amounts described hereinabove, and the progression, remission or stasis of the cardiovascular disease or disorder in the human or veterinary animal is then monitored over time for improvement in the disease or disorder state. If necessary, the conjugate or composition of the invention is then periodically readministered into the vascular system according to dosing and treatment schedules and protocols described herein and others that will be familiar to the ordinarily skilled artisan, until the cardiovascular disease or disorder is cured, prevented or ameliorated. In such embodiments, the conjugates or compositions of the invention are suitably administered to or into the heart, pericardium, vessel or other relevant component of the vascular system parenterally or topically. Parenteral administration is accomplished by any art-known route of administration of a therapy to the vascular system, for example via a route selected from the group consisting of oral ingestion, subcutaneous injection, intradermal injection, transdermal administration or diffusion (e.g., via a salve, ointment, transdermal patch, and the like), intramuscular injection, intravenous infusion, intraarterial infusion, via catheterization, embolization, inhalation, intrathecal administration, implantation of a drug eluting wafer or film, sublingually, or rectally. In such methods, the composition is suitably administered parenterally to the human or veterinary animal in the form of a pill, capsule, solution, suspension or powder that is ingested by the human or veterinary animal, or in the form of a mesh, wafer, film, gel, putty, sphere, nanoparticle or patch that is implanted within the heart, pericardium, vessel or other relevant component of the vascular system at or proximal to the site involved in the cardiovascular disease or disorder.

In other embodiments, administration of the conjugates or compositions to or into the vascular system can be accomplished by any well-known means, including in the form of a solution, an ointment, a salve, a patch, a film, a gel, spheres, nanoparticles, a cream, a topical solution and a drug eluting wafer. For example, the conjugates or compositions can be applied to or introduced into the heart, pericardium, vessel or other relevant component of the vascular system in the form of one or more drops of solution or a suspension that contains the composition or conjugates; via injection; in the form of a coating on a solid material that is implanted into the heart, pericardium, vessel or other relevant component of the vascular system; in the form of a mesh or patch; by attaching the conjugate or composition to, or enclosing it within, one or more nanoparticles that are then delivered into the heart, pericardium, vessel or other relevant component of the vascular system. Other suitable methods of applying the conjugates or compositions to or into the vascular system to accomplish the therapeutic and diagnostic methods of the invention will be readily apparent to the ordinarily skilled artisan.

In related embodiments, the invention provides devices, particularly medical devices, suitable for treating or preventing a disease, disorder or medical condition in a human or veterinary animal suffering from or predisposed to said disease, disorder or medical condition. Such devices suitably will comprise at least one of the compositions of the present invention, in the form of a coating on the device or a composition that is embedded within the device such that it is released from or elutes from the device once implanted within the body of the human or veterinary animal. Suitable such devices include, but are not limited to, artificial joints, stents, catheters, sutures, bone screws, bone plates, prosthetics (e.g., artificial limbs, body structures, organs, etc.), absorbable or non-absorbable meshes, absorbable or non-absorbable patches, drug-releasing wafers, brain neurostimulators (e.g., deep brain neurostimulators), gastric stimulators, cochlear implants, cardiac defibrillators, cardiac pacemakers, insulin pumps, internal infusion pumps, and the like. Suitable other devices useful in accordance with this aspect of the invention will be readily apparent to the ordinarily skilled artisan.

The devices provided by this aspect of the invention are useful for treating, preventing, ameliorating or diagnosing diseases, disorders and medical conditions in humans or veterinary animals suffering from or predisposed to such diseases, disorders or medical conditions. In methods according to this aspect, one or more medical devices of the invention is implanted into the human or veterinary animal, and medical condition of the human or veterinary animal is monitored until the disease, disorder or medical condition is cured, ameliorated or prevented in the human or veterinary animal. Suitable diseases, disorders and medical conditions that may be cured, treated, ameliorated or prevented using the devices and methods of the invention include cancers (such as those described elsewhere herein), and diseases or disorders affecting an organ system of the human or veterinary animal including the integumentary system (particularly diseases or disorders of the skin such as those described in detail herein), the muscular system, the skeletal system (particularly diseases or disorder of the bones, joints, cartilage, tendons or ligaments such as those described in detail herein), the nervous system (particularly those of the brain or the eye (such as anterior segment eye diseases and disorders including but not limited to those involving the corneal nerves (such as corneal pain (which may be acute or chronic), including but not limited to that resulting from damage or injury to the corneal nerves or denervation, e.g., paraocular pain, extraocular pain, and post-herpetic neuralgia), glaucoma, cataracts, vitreous adhesions or floaters, macular degeneration, dry eye syndrome, corneal keratitis, non-infectious corneal ulceration, non-infectious corneal melting, infectious corneal ulceration, infectious corneal melting, conjunctivitis, Stevens-Johnson Syndrome, scleritis, episcleritis, iritis, uveitis, vitritis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, ectasia, keratoconus, corneal laceration, corneal erosion, corneal abrasion, and a post-operative affliction of the eye resulting from eye surgery such as a post-operative cataract surgery state requiring medication or a post-operative glaucoma surgery state requiring medication, or posterior segment eye disorders such as those involving the retina, retinal epithelium (particularly the retinal pigment epithelium), retinal blood vessels, retinal nerves or optic nerve, including but not limited to macular degeneration (wet, dry and age-related), retinitis pigmentosa, retinal tears and detachment, retinopathy (e.g., diabetic retinopathy), retinal arterial or venous occlusion (e.g., BRAO (Branch Retinal Artery Occlusion), CRAO (Central Retinal Artery Occlusion), BRVO (Branch Retinal Vein Occlusion) and CRVO (Central Retinal Vein Occlusion), optic neuritis, optic neuropathy (including, for example, AION (Anterior Ischemic Optic Neuropathy), traumatic optic neuropathy and optic atrophy (e.g., glaucomatous optic atrophy)), and other neuropathies impacting the eye or area around the eye, including paraocular diseases, disorders and medical conditions and extraocular diseases, disorders and medical conditions, such as cranial nerve palsies including but not limited to Cranial III Nerve Palsy, Cranial Nerve IV Palsy, Cranial Nerve V Palsy (e.g., trigeminal neuralgia and post-herpes zoster neuralgia), Cranial Nerve VI Palsy and Cranial Nerve VII Palsy (e.g., Bell's Palsy), the circulatory system, the lymphatic system, the respiratory system (including those diseases or disorders affecting the epiglottis, the trachea, a bronchus, a bronchiole or a lung in the human or veterinary animal, particularly those diseases and disorders described in detail herein), the endocrine system, the urinary/excretory system (including those diseases or disorders affecting the kidney, the ureter, the urinary bladder, the upper urinary tract (i.e., the renal pelvis), the ureter or the urethra of the human or veterinary animal, particularly those diseases and disorders described in detail herein), the reproductive system (including diseases and disorders affecting the testicle, the prostate, the penis, the vagina, the cervix, the uterus, a fallopian tube or an ovary in said human or veterinary animal, particularly those diseases and disorders described in detail herein), the digestive system (including those diseases or disorders affecting the esophagus, stomach, small intestine, colon or rectum in said human or veterinary animal, particularly those diseases and disorders described in detail herein), and nerves or the nervous system (including the peripheral nervous system and the central nervous system, particularly those nerve or nervous system disorders, diseases and injuries described in detail herein). Suitable methods for implanting one or more of the devices provided by this aspect of the invention into a human or veterinary animal, to accomplish the treatment, prevention, amelioration or diagnosis of a disease, disorder or medical or physical condition in the human or veterinary animal will be familiar to the person of ordinary skill in the relevant medical and surgical arts.

Concentrations of the CMPs, or of the CMP-TC conjugates, useful in treating, preventing, ameliorating or diagnosing one or more diseases or disorders according to the methods of the present invention will be readily apparent to the artisan ordinarily skilled in the pharmaceutical and medical arts, particularly in view of the guidance provided herein. For unconjugated CMPs, suitable amounts or concentrations of CMPs to be administered to a subject, particularly a human or veterinary animal, suitable amounts or concentrations of CMPs to be used include those described hereinabove. Based on the guidance provided herein, one of ordinary skill in the medical, pharmaceutical and/or pharmacological arts can determine the appropriate amount of the conjugates and compositions of the invention to be used per kilogram (kg) of body mass of the human or veterinary animal. For conjugated CMP-TCs, the same amounts or concentrations of CMPs described herein, whether in concentration (e.g., ng/ml or μg/ml) or in amount (e.g., mg per kg of body mass), are suitably administered to the subject, and the amount of active pharmaceutical ingredient or biologic is calculated during the conjugation process to deliver therapeutically effective amounts of the desired active pharmaceutical ingredient or biologic, depending upon the disease or disorder that is to be treated, prevented, ameliorated or diagnosed in the human or veterinary animal. Suitable amounts or concentrations of active pharmaceutical ingredients or biologics to be used according to this aspect of the invention will be familiar to the ordinarily skilled artisan, and can be readily determined from information contained herein and other information that is available in the relevant arts.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1: Effect of CMPs and CMP-TC Conjugates in Treating Nervous System Disorders

To examine the possible therapeutic effects of CMPs of the invention in treating nervous system disorders, studies were designed to test the ability of certain CMPs conjugates to stimulate nerve growth in various in vitro settings.

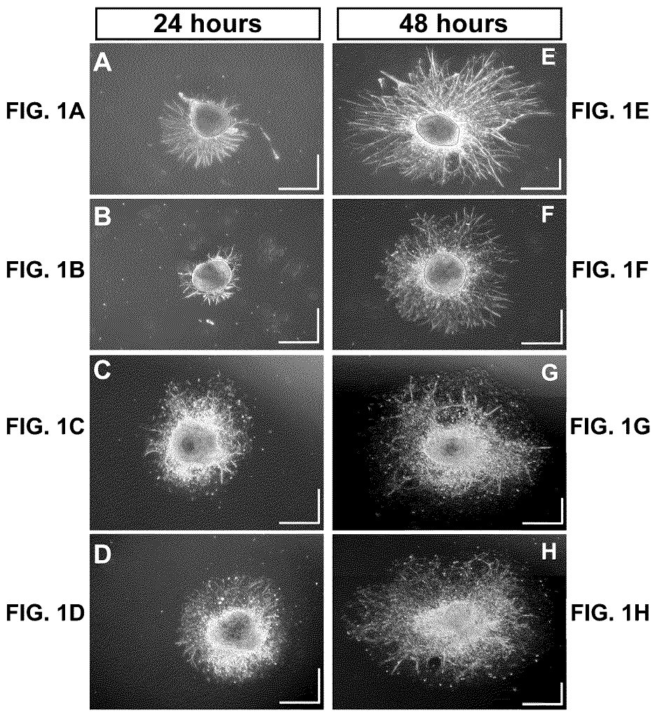

In initial such studies, dorsal root ganglion (DRG) neurons, a component of the peripheral nervous system (Berta, T et al., Expert Opin. Ther. Targets 21:695-703 (2017)), were isolated from day 19 embryonic rats and plated onto tissue culture plates that had been coated with intact collagen or with MMP-1-digested collagen and then treated with vehicle (PBS) or with one of several different CMPs of the invention: CMP 05A (SEQ ID NO:3), CMP 09C (SEQ ID NO:5), CMP 10A (SEQ ID NO:473) or CMP 13A (SEQ ID NO:6), each at 100 μM, 100 μL per well, and incubated at 37° C. for five hours. Plates were then rinsed with culture medium, and DRG neuronal cells were plated uniformly on plates having one of three surface coating conditions: (1) intact collagen; (2) MMP1-digested collagen; and (3) MMP1-digested collagen+CMP. Plates were then incubated at 37° C. for 24 hours or 48 hours, and then examined for morphology and neurite outgrowth by inverted brightfield microscopy. Representative results are shown in FIG. 1.

After 24 hours, montages of collapsed z-stacks of phase contrast micrographs of DRG explants on intact collagen showed modest neurite outgrowth, which increased substantially by 48 hours (FIGS. 1A,E). Plating on damaged collagen reduced the extent of outgrowth FIGS. 1B,F), while explants plated on damaged collagen treated with two distinct CMPs (CMP 09C or CMP 10A) demonstrated improved outgrowth (FIGS. 1C,G and FIGS. 1D,H).

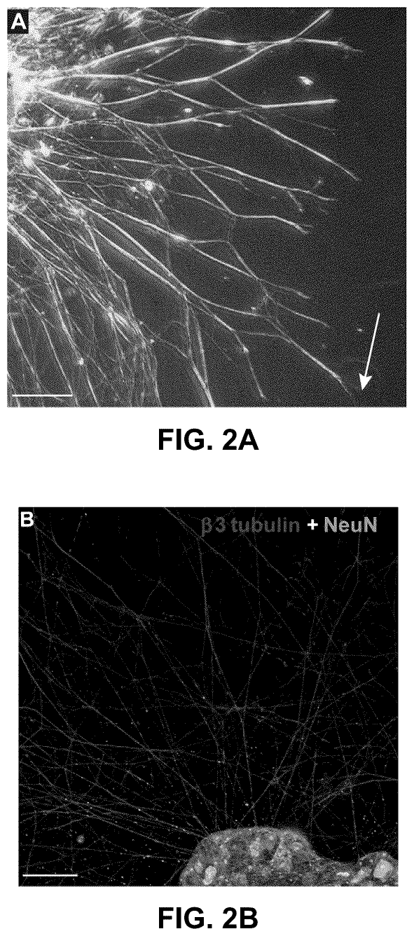

To measure neurite outgrowth accurately, explants were visualized live using multifocal high-magnification phase contrast microscopy to optimize identification and measurement of the longest neurite (FIG. 2A, white arrow) and the size of the DRG explant growth field. This method was preferred to post hoc measurement of neurites visualized post-fixation using antibodies against 33 tubulin (FIG. 2B).

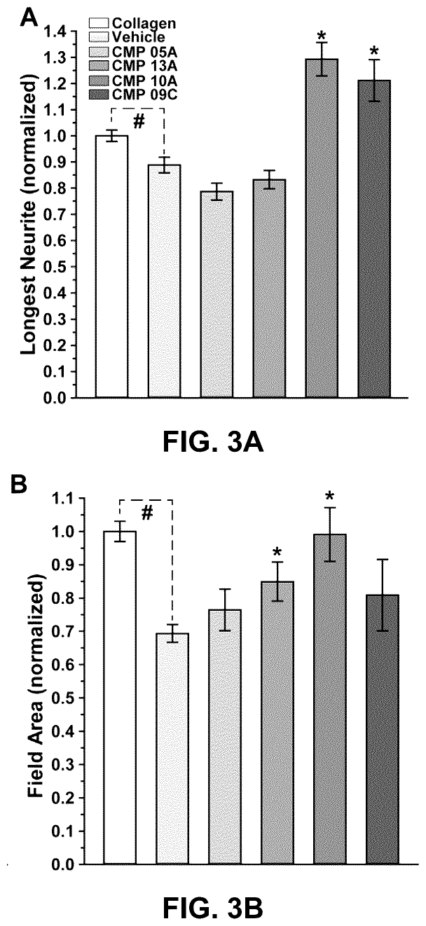

These trends were quantitated by measuring both the longest neurite extending from each explant and the area of the field extending from and surrounding the explant with neurite coverage, both normalized to their values measured under conditions of intact collagen. Compared to intact collagen, the longest neurites of vehicle-treated explants plated on MMP1-digested collagen were 15% shorter (p=0.014; FIG. 3A). In contrast, two CMPs of the four tested in this condition induced neurite outgrowth that was 29% (CMP 10A) and 21% (CMP 09C) greater than even intact collagen (p<0.036). Similarly, vehicle-treated explants demonstrated 30% smaller growth field areas compared to explants on intact collagen (p<0.001; FIG. 3B). Again, two CMPs significantly improved outgrowth compared to vehicle (p≤0.03), one of which (CMP 10A) also induced greater neurite outgrowth.

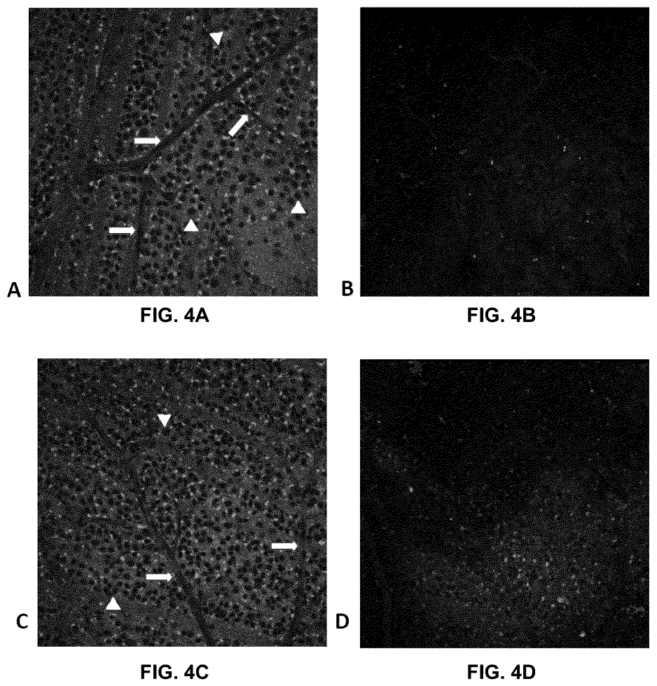

These results are reminiscent of those presented previously by some of the present inventors (see U.S. Patent Publ. No. US 2020/0353056 A1, the disclosure of which is incorporated herein by reference in its entirety). In those previous studies (presented here in FIG. 4), a fluorescently tagged CMP injected intravitreally or applied dropwise into mouse eyes demonstrated highly directed binding at and around ganglion cell neurons in the ganglion cell layer of the retina (FIGS. 4A, 4C), with numerous blood vessels (arrows) and ganglion cell nuclei (arrowheads) clearly visible in contrast. The fluorescence was seen to be localized in the ganglion cell layer, with very little of the CMP localized to the inner limiting membrane of the vitreous surface (FIGS. 4B, 4D). Taken together, these results demonstrate that the CMPs of the invention localize to areas of collagen damage at or near neurons and ganglia, and suggest that this localization results in repair of the ECM in those areas thereby promoting neurite outgrowth and regeneration of neuronal and neuronal process (dendritic and axonal) growth and regeneration, ultimately perhaps resulting in the regeneration and protection of neurons, neuronal processes and nerves in the peripheral and central nervous systems.

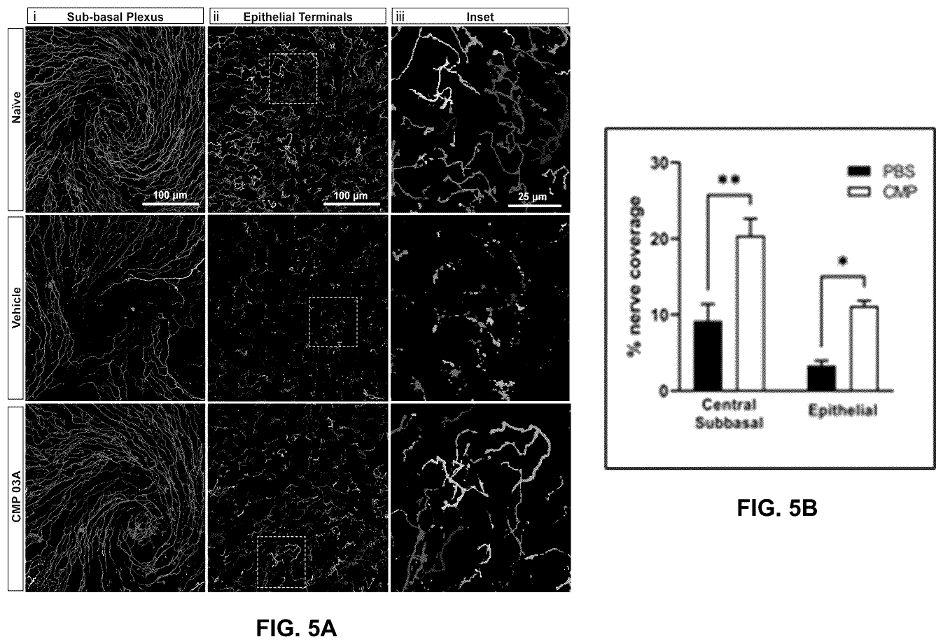

As another means of examining the effect of CMP treatment on neuronal structure and activity, the ability of CMPs to reverse and repair nerve fragmentation in the corneal nerve bed was examined. C57BL/6 mice were treated bilaterally with topical 1% atropine 4× daily with desiccation for a total of 7 days, and at day 7 mice were mice were treated topically with application of vehicle (PBS) or CMP 03A (SEQ ID NO:1) (200 mM), 1× per day bilaterally for a further 7 days. To assess the extent of nerve degeneration, fragmentation of βIII-tubulin-labeled nerve fibers was determined using cholera toxin B (CTB) staining of axons and axonal fragments in the various layers of the cornea. The number of contiguous nerve fragments in each sub-basal and epithelial terminal image were determined by confocal microscopy. Images were acquired in z-stacks through the entire central cornea using an Olympus FV1000 confocal microscope at 800 px²×800 px² resolution on a 40× objective. Due to the curvature of the flattened cornea, different regions of the same corneal image had sub-basal nerves and epithelial nerve endings in different z-planes and thus needed to be stacked separately to compose the flattened final image. To aid in segmentation, a Python script was utilized to split the image into nine smaller z-stacked tiles that could be processed individually. The bounds for each tile sub-stack were manually chosen then flattened using the standard deviation z-stacking method in ImageJ. This produced a single stacked image each for the sub-basal plexus and epithelial terminal plexus. Each image was manually thresholded in ImageJ and the binary image saved. The python script re-stitched the binarized, flattened smaller tiles to give final stacked images of both the sub-basal nerve plexus and epithelial endings. The complete binarized images were then processed using a Python script that performed a flood-fill algorithm on every contiguous region, or fragment, identified in the binary image. Each unique, contiguous fragment was randomly assigned a red-green-blue (RGB) value, and an image generated to visually assess nerve fragmentation. The number of unique RGB values generated in this way was saved as a record of the number of fragments in each image. Representative pseudo-colored fragmentation images are shown in FIG. 5A, where each contiguous nerve fragment is identified by a unique color. In naïve mice, sub-basal nerve fibers in the central swirl region were formed by long, contiguous fragments (FIG. 5A, upper row left). Similarly, epithelial terminals, although numerous, were intact; each nerve terminal was mostly unicolored (FIG. 5A, upper row center and right (inset)). In the vehicle group, there were a larger number of shorter, contiguous nerve fragments at the sub-basal plexus (FIG. 5A, center row left), suggesting discontinuity. Compared to naïve animals, epithelial nerve terminals were reduced in number and highly fragmented (FIG. 5A, center row center and right (inset)), indicating extensive degeneration. Interestingly, CMP treatment reversed this trend; nerves appeared to have greater continuity and were much less fragmented at the sub-basal (FIG. 5A, bottom row, left) and epithelial levels (FIG. 5A, bottom row, center and right (inset)), and exhibited levels of fragmentation similar to naïve mice (FIG. 5A, top row). CMP treatment was found to significantly improve the outgrowth of nerve fibers in comparison to vehicle in both the central sub-basal and epithelial layers (FIG. 5B).

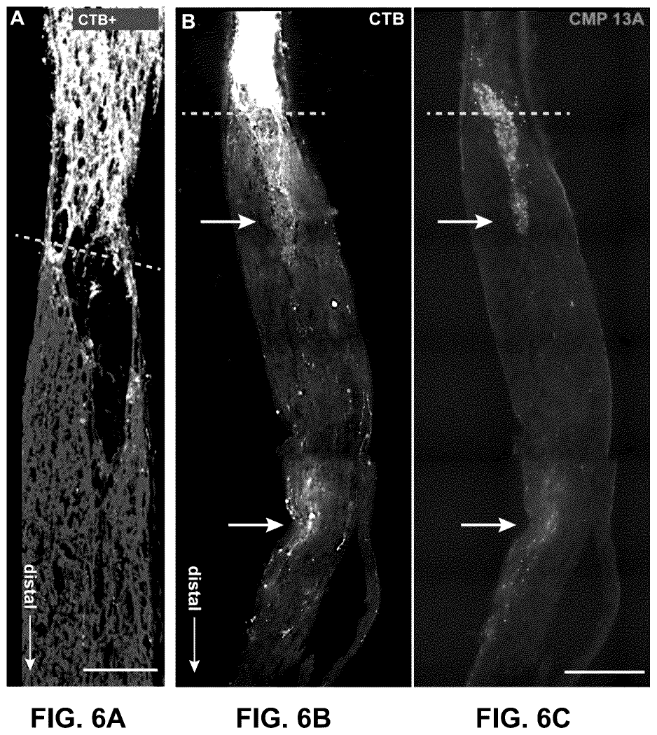

To further examine the effects of CMPs on nerve regeneration, optic nerve crush experiments were performed. This approach is a useful model of traumatic optic neuropathy and for assessing potential neuroreparative and neuroregenerative strategies (Cameron, E G et al., Bio-protocol 10(6) (Mar. 20, 2020), doi: 10.21769/BioProtoc.3559). Axon degeneration in this injury model also involves degradation of matrix collagen associated with robust tissue remodeling (Bernardo-Colon, A et al., Cell Death Dis. 9:1097 (2018); Sharma, T P et al., Mol. Neurodegener. 9:14 (2014)). Mice were subjected to retrotubular nerve crush, and then three days later received an intravitreal injection of vehicle (DMSO) or of fluorescently tagged CMP 13A (SEQ ID NO:6). Optic nerves were longitudinally sectioned in the site of the injury two weeks following nerve crush, and sections were examined for axon length (via cholera toxin B labeling) and CMP localization (via fluorescence microscopy). Sections were also labeled with glial fibrillary acidic protein (GFAP) to localize astrocyte glia in the sections. Results of these studies are shown in FIG. 6. As seen in FIG. 6A, axons containing CTB (false color white) from mice treated with vehicle were seen to extend to the site of the crush (dashed line), but generally not beyond it distally toward the brain. In contrast, CTB-containing axons in a section of nerve from eyes receiving CMP treatment extended beyond the crush site (FIG. 6B, dashed line) and were apparent even at more distal locations along the nerve (FIG. 6B, arrows). Repaired axons were largely coincident with localized patches of CMP (FIG. 6C, arrows). Scale=200 μm (FIG. 6A) or 100 μm (FIGS. 6B,C).

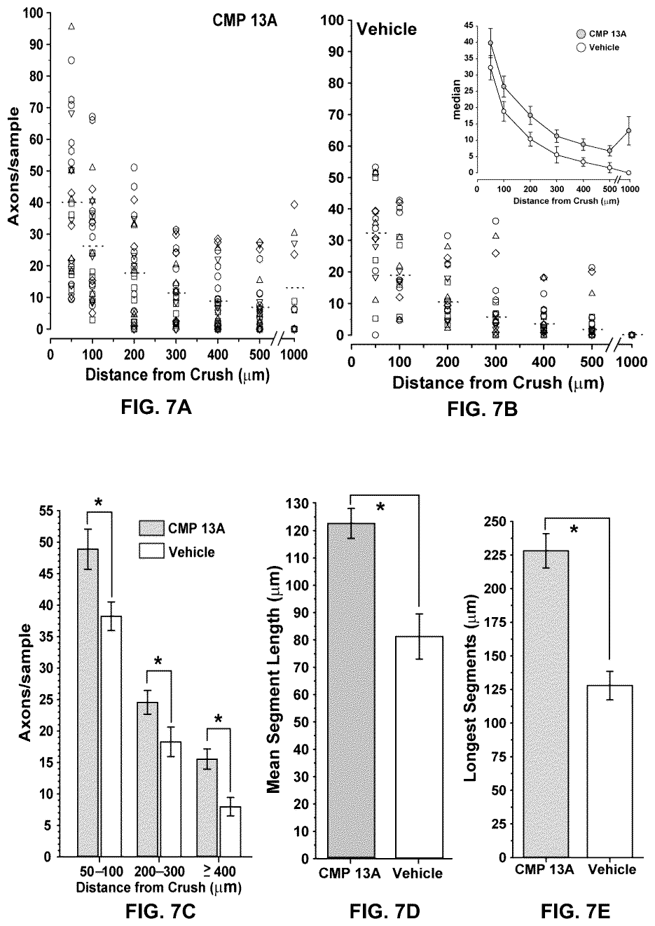

Next, we quantitated the number of CTB+ green axon segments at discrete distances distal from the crush site (towards the brain). Results are shown in FIG. 7. Nerves from eyes receiving CMP 13A (SEQ ID NO:6) demonstrated more axon segments than nerves from vehicle eyes at each location examined, with some samples having CTB+ segments as far as 1 mm distal from the injury (FIGS. 7A,B). Even so, in both cohorts, there were a number of samples with few, if any, axons distal from the crush site, leading to substantial variability (FIG. 7B, inset). To better estimate the magnitude of the effect due to CMP 13A compared to vehicle, we pooled the measurements above the median at each location into three bins: 50-100 μm, 200-300 μm, and 400 μm and further from the injury site. For these locations, nerves from CMP 13A-treated eyes demonstrated a 29%, 39%, and 96% greater abundance of CTB-containing axon segments, respectively (FIG. 7C, p≤0.02). Intact axon segments in these nerves were also longer than those in nerves from vehicle-treated eyes. The mean length of intact axon segments in nerves from CMP 13A-treated eyes was 51% greater than that in the vehicle cohort: 122.6±5.5 vs. 81.1±8.3 μm (p<0.001; FIG. 7D). Similarly, the mean for the longest 25 segments in the CMP 13A nerves was nearly 80% greater than that for vehicle nerves: 228.1±12.9 vs. 127.8±10.5 μm (p<0.001; FIG. 7E).

### Discussion

The importance of the ECM in physiology is reflected in its evolutionary age. A key step in the evolutionary transition from unicellular to multicellular organisms some 600 million years ago was the emergence of genes coding for material components of the ECM necessary to provide a platform allowing cells to work as a unit and form tissues (1, 2). Its abundance also reflects the diverse physiological roles of the ECM, which provides structural integrity, mediates extracellular signaling capabilities, and facilitates specialization in all tissues (3). Indeed, as cells differentiate and associate with other cells to form specialized structures, the composition of the ECM scaffold differentiates in kind to provide tissue-specific support (4). The importance of ECM in multicellular organization is evident very early in development, as genes encoding highly conserved ECM proteins are expressed in stem cells as early as the 16-cell stage in the growing embryos of many higher eukaryotes (5, 6). During the development of the CNS, ECM production is regulated spatially and temporally to drive neurogenesis, neural cell migration, and axon growth and guidance (7). For example, in the visual system, ECM-cell communication is required to drive the connection of retinal-derived axons in the optic nerve to terminal zones in the brain (8).

Far from simply being a substrate for tissue growth and support, the ECM also acts as a biochemical reservoir of signaling molecules, allowing cells and tissues interacting with the ECM to adapt to environmental cues and stressors (1, 9). Synthesis and release of ECM components and cell-ECM communication is an integral part of numerous biological processes including stem cell maintenance and differentiation (10), innervation (11), angiogenesis (12, 13), and wound healing (14). Cells respond not only to the chemical composition of the ECM itself but also to its mechanical properties (15). The ECM responds through biochemical and biomechanical signals to the resident cells of the tissue in a process called ‘dynamic reciprocity’ or ‘bidirectional crosstalk’ (16, 17). The bidirectional relay of ECM-cell signaling occurs during tissue homeostasis and in pathological conditions (1). Cells therefore must sense and also regulate ECM mechanics during homeostasis to promote the structural integrity and healthy functioning of the ECM itself (15). Homeostasis of the ECM is maintained by cells sensing mechanical loads acting on the tissue and transducing these mechanical signals through the ECM. This process of mechanical sensing is governed through ECM proteins such as collagen and elastin which are built to withstand and respond to mechanical stretch and strain (15). Thus, the ECM represents a highly conserved, evolutionarily critical driver of tissue specificity, connectivity, and adaptation.

### The Central Nervous System and Collagen

The complex interplay between ECM and resident cells is reflected and exemplified in the CNS, where ECM represents about 20% of total brain mass (18). Both neurons and glial cells in the CNS integrate dynamically with ECM to maintain tissue homeostasis. Although neurons express and secrete various ECM components (19), astrocyte glial cells are integral to the deposition of ECM and help to maintain ECM function and integrity as the CNS ages (20). For example, with increasing age, the structure of the ECM in the CNS changes to regulate synaptic plasticity (21). The ECM in the CNS predominantly comprises fibrous proteins (including collagens and elastin) and glycoproteins (including proteoglycans, glycoproteins, and laminins) that together form a three-dimensional medium through which complex networks of cells can communicate. Smaller homo- and hetero-polymers bind to form supramolecular assemblies with binding domains for growth factors, cytokines, and cell adhesion molecules (22). In this way, the ECM serves as a medium capable of not only conveying but also binding and releasing ligands.

Proteoglycans are highly abundant in neural tissue and form the basis of high-order ECM structures around cells. Proteoglycans in neural tissue are rich with covalently-bound glycosaminoglycans (GAGs)—long chains of charged polysaccharides (sugars). The major GAGs include heparin sulfate, chondroitin sulfate, hyaluronan, and keratin sulfate (7). Even so, collagen is the most prevalent and integral component of the ECM. There are nearly 30 types of collagens, and their most highly recognized role is to provide tissues with structural and mechanical integrity. In the human body as a whole, collagen is the most abundant protein, in particular types I and III (23). Collagen renders both biological stiffness and strength to tissues (including the ECM), influencing the degree to which stress deforms tissue and the maximum stress that can be applied before breakdown (15, 23). Several members of the collagen superfamily, particularly collagens I, IX, and XVIII, are involved in development of the CNS, playing important roles in neuronal maturation, neural circuit formation, axon guidance, and synaptogenesis (24-26).

The ECM of the CNS is not a uniform, homogeneous sea filling in between neuronal, vascular, and glial elements. Rather, it comprises structurally distinct and specialized landscapes whose structure reflects the function of the surrounding elements. These begin with the non-fibrillar but protein-rich basal lamina. Basal lamina acts as a macromolecular sieve-like barrier between tissues, shielding cells from unwanted biochemical and biophysical stressors while also providing a medium for intercellular communication (27, 28). Basement lamina is produced by microcapillary endothelial cells, astrocytes, and pericytes (29). Biochemically, the basal lamina contains four major ECM proteins: collagen IV, laminin, nidogen, and heparan sulfate proteoglycans (HSPGs) (29). Minor constituents include fibulins, osteonectin, and netrin-4 (30). It assembles near cell surfaces—composed mainly of interconnected polymers of collagen IV and VII, with areas bound to laminin and other glycoproteins (31, 32). Collagen IV, which is produced by endothelial cells, astrocytes, and vascular pericytes, creates a main structural scaffold to which other ECM-associated proteins can bind and interact (28, 31). The basement lamina is also found in the peripheral nervous system but differs from the CNS basement lamina due to the absence of astrocytes.

Structural proteins, including fibrillar collagens, were thought only to exist along blood vessels and meninges of the brain in healthy tissue. Further, it was thought that post-development, collagen secretion in the CNS by astrocytes, neuronal cells, and other glial cells was suppressed (26). However, the presence of collagens in healthy human brain parenchyma (including in neurons) counters these presumptions (33, 34). As well, in the aging human brain, both neurons and astrocytes express genes encoding a range of collagen types (35, 36). Although not a major component, collagen appears to have an important role in the structure and physiology of perineuronal nets (PNNs). Densely packed PNNs, which were wrongly identified as artifacts in Cajal's silver staining, are condensed ECM surrounding the soma and the proximal/middle dendrites of neurons and often extend to include the axon initial segment. In the brain, they exist in multiple regions including the hippocampus, cerebral cortex, cerebellum, and basal ganglia (35). Synapses within these regions are surrounded by and embedded in the PNN (37). Loss of collagen XIX leads to a reduction in PNN formation in the mammalian telencephalon (38), and collagen within hippocampal PNNs is associated with the formation of long-term memory (39). After focal ischemia in the brain, upregulation of collagen IV is evident in PNNs (40).

Dynamic Collagen: More than a Scaffold

Though fulfilling its biological role as a scaffolding protein in the ECM, collagen is nevertheless highly dynamic throughout its lifecycle, interacting with molecular binding partners by forming a complex known as the collagen interactome (41). During collagen biosynthesis, interactions with enzymes such as hydroxylase and lysyl oxidases as well as chaperone proteins ensure that collagen adopts its triple helical structure as it integrates into the matrix (42, 43). Once fully formed and integrated, collagen interacts with a number of other ECM components such as fibronectin, proteoglycans, GAGs, and heparin (42). Collagen also binds multiple cell-surface receptors, including integrins (44), osteoblast receptors (e.g., OSCAR) (45), mannose receptors (46), and discoidin domain receptors (DDRs) (42, 47). During turnover or breakdown, collagen also interacts with matrix metalloproteinase (MMP) enzymes which help facilitate its degradation or digestion (48). Thus, MMP activity is by necessity a tightly controlled process. During disease or with increasing age, an imbalance in MMP activity (i.e., increased or decreased beyond native collagen turnover requirements) can lead to excessive matrix collagen deposition or, in the other direction, degradation (49, 50).

Collagens are multi-domain proteins having at least one triple-helical domain that can comprise most of its structure, as with collagen type I, or a much smaller fraction, as with collagen XII (51). Many other proteins with relevance to the CNS contain collagen-like domains. The conformational state of collagen determines its binding affinity for specific ligands. Some ligands favor binding to the native triple helical collagen structure, with no affinity for fragmented collagen or smaller collagen fragments (41, 52). For instance, some ligands including integrins, DDR1/2, MMP1, and chaperone heat shock proteins such as Hsp47 (53) bind only to the native helical structure of collagen (41). The collagen-binding site of DDR receptors recognizes specific surface triple helical sequences on fibrillar collagen (54) thought to be mainly obscured in the native structure of collagen to prevent over-activation of downstream signaling cascades (47, 55). DDR1 is activated by both fibrillar and non-fibrillar collagens (types I to VI) whereas DDR2 is only activated by fibrillar collagen (types I and III) (56). Integrin receptors lie on the outer surface of collagen (57). However, unraveling of collagen strands exposes RGD sequences in collagen that bind to specific types of integrin e.g., αvβ (58-60). The molecular chaperone Hsp47 binds to only a few GXR motifs in collagen, with most of the HSP47 binding sites located near the N-terminal part of the triple-helical region (61). Interestingly, MMPs recognize native collagen but have intrinsic helicase activity that unwinds the triple helical structure to expose MMP binding sites for the cleavage of collagen (62). Other proteins, such as lysyl oxidase, preferentially bind to higher-order structures such as fibrils (63, 64). In conditions where collagen becomes denatured or damaged, proteins involved in collagen turnover recognize epitopes exposed in the denatured or degraded collagen (41, 48). Similarly with amyloid precursor protein (APP); binding of APP to collagen I occurs in native and damaged states and appears to be competitive with heparin—this suggests an overlap between the binding site for APP and heparin (65).

The tightly wound triple helical structure of collagen renders it weakly antigenic (the ability to bind to antibodies) and largely non-immunogenic (the ability to induce an immune response through antibodies (66)). However, the biological reactivity of collagen with cell surface antigens and receptors is becoming increasingly recognized as critical to disease states (41). Changes to collagen structure, such as damage or increasing turnover during disease, can alter its antigenicity and immunogenicity (67, 68). Increasing collagen turnover, as well as outright collagen damage, is evident during inflammation in disease and may represent an important therapeutic target in neurodegenerative CNS disorders (69).

### The Collagenous ECM Landscape as a Driver of CNS Disease

The ECM plays critical roles as a biologically active scaffold for maintaining biophysical stability and structure and as a mediator for the diffusion and availability of signaling molecules, such as those mediating interactions between axons and astrocytes (3). Cells of the CNS modify the production and excretion of ECM components in response to environmental cues that include oxygen or nutrient concentrations and biochemical and mechanotransducive signals. In this sense, regulation of ECM turnover is critical to the function and survival of neurons in the CNS.

The extracellular landscape of the CNS evolves through changes in activity of both ECM-related genes and enzymes in both disease and aging (70). Cellular senescence is emerging as a key contributor to neurodegenerative diseases of the CNS. Cells that become senescent have a secretome that includes cytokines and chemokines, as well as ECM, that can signal to surrounding tissue (76). Outside of the CNS in fibrotic diseases, the ECM—including collagen—can regulate cellular senescence (77). Alterations in ECM occur across the spectrum of CNS diseases including Alzheimer's Disease (71, 72), Parkinson's Disease (73, 74), and of special interest to the authors, optic neuropathy (75, 76). Of relevance to Alzheimer's, amyloid-beta (Aβ) peptides contain collagen type XXV (also known as collagen-like amyloidogenic component), which influences amyloid fibril elongation (77), and there is a genetic association between this collagen and Alzheimer's in some populations (78). Finally, type I collagen contains binding domains for amyloid precursor protein that contribute to monocyte recruitment in disease states (79). Conversely, collagen VI expression in the brains of hAPP mice and individuals with Alzheimer's disease may be neuroprotective; increased expression of collagen VI in neurons was protective against Aβ toxicity (80).

Astrocyte glial cells in the CNS are key players in the maintenance of ECM. During disease and with aging, astrocyte physiology moves towards a more reactive profile and changes in the deposition of ECM occur (81). Collagen is intricately involved in this process, which includes overproduction, degradation, and altered composition detectable in pathophysiological samples (70). Changes in the structure and/or deposition of collagen impact cellular signaling and tissue biomechanics which in turn can alter cellular responses in tissue, driving disease and inflammatory states (69). Collagen contains multiple binding sites that serve as ligands for both cell surface receptors and signaling pathways involved in inflammation. MMP-induced disruptions of these binding sites lead to chronic inflammation (82, 83). Thus, even slight changes in ECM early on in disease progression may be overlooked as drivers of neurodegeneration.

CNS neurodegenerations often coincide with vascular pathologies such as compromised blood flow and leakage of the blood-brain barrier (81). In systemic vascular diseases such as atherosclerosis, damage to ECM in vascular walls contributes to the progression of disease (84). Atherosclerosis is also associated with increased vessel stiffness possibly due to large deposits of collagen (84). Recently, a potential role for complement factors C3 and C4 and ECM in the vascular etiology of neurodegenerative diseases has emerged. The unexpected observation that C3 and C4 deposit in vessel walls and colocalize with collagen has unearthed a potential interaction between complement proteins and collagen in disease and with age (85). In CNS diseases such as Alzheimer's disease, early microglial activation is thought to be an early driver of neurodegeneration (86, 87). Activated microglia promote phagocytosis of neurons and also contribute to the breakdown of the blood-brain barrier (87). One mechanism for microglial activation depends on the binding of C3 to C3 receptors present on the microglial cell surface (88). Thus, damaged collagen present in blood vessels due to aging or disease may act as a novel C3 reservoir, which may trigger early microglial activation and drive neurodegeneration.

The CNS lacks the intrinsic capability to regenerate, in part due to microenvironmental factors that increase local inflammation and reactive gliosis (89). Pathological changes in ECM collagen may contribute to the limited capacity for regeneration in the CNS through perturbed signaling. A major component of astrocyte-mediated ECM deposition is collagen, which may promote ECM deposition and impact regeneration in the CNS (90). As matrix collagen degrades, the formation of a glial, fibrous complex by reactive astrocytes secreting an overabundance of collagen type IV creates a barrier to axon repair and regeneration in the CNS by concentrating inhibitory molecules like proteoglycans and semaphorins and inducing migration of inflammatory microglia and other immune cells (91-94). Thus, this hypertrophic glial complex (typically called a glial scar) provides not only a biomechanical barrier to regeneration but a biochemical one as well (95). In optic nerve degeneration, changes in collagen alignment and stiffness in the eye's sclera (which shapes the eye) and in the head of the optic nerve through which axons pass on the way to the brain impact progression through increased inflammation (96).

In contrast to the CNS, the peripheral nervous system has a far greater capacity for neuronal repair. Molecular signaling pathways such as integrins play an important role in the spontaneous regeneration of peripheral axons (97). Collagen is also highly upregulated after peripheral nerve injury and synthesized by Schwann cells and fibroblasts (98, 99). The high levels of collagen at the site of peripheral nerve injury could facilitate important axonal integrin signaling required for regeneration and may be indicative of a more important role of collagen than previously appreciated. Regeneration in non-mammalian species such as the zebrafish is strongly ECM-dependent; conditions that favor regeneration are rich in numerous collagens, including collagen XII, suggesting a pro-regenerative capacity for certain collagen sub-types (100, 101). Current regenerative strategies have focused on modulating growth factor signaling, regeneration-associated genes, glial-mediated axon regeneration, cell replacement, and peripheral nervous tissue grafting (81), all with limited success. None leverage the complex signaling capabilities of the ECM and in particular, collagen.

Throughout the body, including in the CNS, the biomechanical properties of tissue can impact cellular signaling and cell recruitment to the site of damage or disease. After an injury in the CNS, the biomechanical stiffness of the tissue in the brain and spinal cord decreases, correlated with increased levels of ECM components including collagen IV and laminin (95). If collagen becomes damaged enough to alter the stiffness of the local tissue area, a wave of mechanically induced signaling involving other cells could ensue. For example, traumatic injury to the brain involves a transient period of rapid neovascularization, increased vessel permeability, and accumulation of pro-angiogenic factors likely released by microglia cells (102). These same cells likely contribute to tissue remodeling following injury through the secretion of proteases. The vascular basement membrane in the brain contains the heparan sulfate proteoglycan collagen XVIII, among other collagens, which contains a 20-kDa anti-angiogenic endostatin fragment (reviewed in (103)). Subsequent to the injury, parenchymal accumulations of collagen XVIII/endostatin accompany increased numbers of microglia expressing the same (104). This collagen XVIII-dependent process could contribute to counteracting the early angiogenic injury response to limit secondary injury.

### Repairing Collagen in the ECM as a CNS Therapeutic

For CNS regeneration, as aforementioned, tissue biomechanics can impact the capacity for neuronal axon growth and guidance. Thus, maintaining intrinsic ECM biomechanical properties in itself may be a potential therapeutic avenue. Just as damage to the ECM is prohibitive to regeneration, therapies that rebuild the matrix hold great therapeutic potential to promote neural repair and regeneration across conditions including traumatic brain injury (105, 106). This is especially so for therapies utilizing or mimicking types I and IV collagen, which are known for very low antigenicity and robust bioavailability (107, 108). Since the collagen interactome and tissue reactivity is heavily governed by collagen structure in the ECM, a more prominent role of collagen in tissue ECM homeostasis is emerging. These observations open a potential therapeutic avenue for matrix repair in CNS disease. Indeed, a promising area is the potential uses of ECM and ECM-derived peptides that improve neuronal regeneration and functional recovery (109). Similarly, implantation of collagen-rich scaffolds following brain surgery reduces microglial activation and inflammatory-related cytokines (110).

The signature characteristic of collagen is its triple helical structure—a set of three polypeptide chains comprising repeating sequences of glycine-x-y triplets where x and y often (but not always) represent proline and hydroxyproline (111). Recent work from our laboratory and others supports the idea that rebuilding triplets damaged by protease activity can repair CNS tissue and promote neuronal survival in a broad spectrum of neurodegenerative conditions. For example, following optic nerve crush, injection of a collagen mimetic peptide (CMP) that intercalates into and reforms compromised triple helices promoted axonal outreach beyond the crush site and extended the length of intact axon segments (112). Similarly, following the induction of optic nerve degeneration by elevated ocular pressure, CMP treatment restored functional axon transport to central brain targets (112, 113). This is a critical finding, since degradation of axonal transport presages outright axon degeneration (114, 115). CMPs have a protective and reparative influence on peripheral nerve damage as well, demonstrating trophic capacity for dorsal root ganglia (DRG) challenged by MMP-induced degradation of ECM and for the corneal nerve bed damaged by surface desiccation (81, 113). Gels enriched with collagen type I similarly promote neurite extension from DRG explants (116). The reparative influence of collagen segments is not limited to neurons. Segmented sequences of collagens IV, XV, and XVIII promote the growth of blood vessels and tumor cells and influence a variety of other cellular activities (117). Synthesized CMPs target areas of collagen disruption associated with skin wounds by reforming the native triple helix through intercalating into disrupted collagen (118, 119).

There are multiple mechanisms through which repairing damaged triple helices in collagen could affect a therapeutic influence. Due to its intrinsic structural properties, collagen confers significant amounts of stiffness to tissue, in concert with other constituents of the ECM (120). Interestingly, a stiffer matrix reduces the potential for astrocyte reactivity and gliosis, which presage the formation of the inflammatory complex at CNS injury sites that is so inhibitive of regeneration (121). Along these lines, unlike other tissues that scar, brain and spinal cord demonstrate diminished elastic stiffness after acute injury, which coincides with increased astrocyte reactivity and inflammation even distal to the injury site (95). In this way, repaired collagen leading to stiffer matrix could help reduce the inflammatory response at the level of the macroenvironment in the neuronal milieu. At the microenvironment level, degradation of collagen by MMP activity can disrupt binding sites that otherwise inhibit local inflammatory signaling complexes. For example, collagen types I and III (and possibly IV) contain high-affinity binding sites for LAIR-1 (leukocyte-associated immunoglobulin-like receptor), which is expressed by most hematopoietic cells to attenuate their activation (122). Intact triple helices in collagen crosslink LAIR-1 to inhibit immune cell activation, while reduced LAIR-1 binding sites in disrupted collagen has the opposing effect (82). Interestingly, the sequence for the binding site for LAIR-1 (also called CD305) is conserved between collagen and the complement component C1q, the initiator of the classical complement pathway at the root of innate and adaptive immunity (123). C1q mediates synapse elimination from dendritic arbors in neurons and is activated early in diseases such as Alzheimer's (124). Collagen and C1q partner to locally regulate LAIR-1 to avoid immune dysfunction. Similarly, intact collagen type I inhibits the secretion of interleukin-8 from neutrophils through interaction with integrin receptors (125). The threshold for a local inflammatory response is breached with shortening of collagen strands through degradation in disease or injury (83).

In summary, CNS ECM represents more than a passive scaffold to support tissue; ECM is a diverse, dynamic, and highly bioactive substrate with roles in cellular biomechanics and differentiation, as well as cellular signaling. Although ECM collagen has distinct roles in tissues outside of the CNS, its integral role in the CNS during homeostasis and disease has been overlooked. Here we highlight multiple actions of collagen, at the macroscale (in tissue support and biomechanics) and microscale (ligand binding capacity) in the CNS. The structure of collagen is important in its highly functional nature. Damage to CNS collagen is evident in disease and with aging and has major implications for driving neurodegeneration through its impact on inflammatory pathways. Repairing damaged collagen in the CNS using mimetic peptides represents an exciting new therapeutic avenue for neurodegenerative diseases, where the restoration of collagen structure at the microlevel could help to repair, protect, and even regenerate CNS neurons. Together, these results indicate that CMPs of the invention are useful in enhancing the repair of a disrupted collagen matrix to thereby promote the proliferation, migration and network formation in neuronal cells. As a result, the CMPs and CMP-containing compositions, and methods of their use, provided by the present invention should prove useful in treating, ameliorating, preventing and diagnosing a variety of diseases and disorders involving nerve cells and the nervous system.

### REFERENCES

1. Hussey, G. S., J. L. Dziki, and S. F. Badylak, Extracellular matrix-based materials for regenerative medicine. Nature Reviews Materials, 2018. 3(7): p. 159-173.
2. Ozbek, S., et al., The evolution of extracellular matrix. Mol Biol Cell, 2010. 21(24): p. 4300-5.
3. Barros, C. S., S. J. Franco, and U. Muller, Extracellular matrix: functions in the nervous system. Cold Spring Harb Perspect Biol, 2011. 3(1): p. a005108.
4. Bonnans, C., J. Chou, and Z. Werb, Remodelling the extracellular matrix in development and disease. Nat Rev Mol Cell Biol, 2014. 15(12): p. 786-801.
5. Zagris, N., Extracellular matrix in development of the early embryo. Micron, 2001. 32(4): p. 427-38.
6. Leivo, I., et al., Appearance and distribution of collagens and laminin in the early mouse embryo. Dev Biol, 1980. 76(1): p. 100-14.
7. Bandtlow, C. E. and D. R. Zimmermann, Proteoglycans in the developing brain: new conceptual insights for old proteins. Physiol Rev, 2000. 80(4): p. 1267-90.
8. Su, J., et al., A cell-ECM mechanism for connecting the ipsilateral eye to the brain. Proc Natl Acad Sci USA, 2021. 118(42).
9. Krishnaswamy, V. R., et al., Demystifying the extracellular matrix and its proteolytic remodeling in the brain: structural and functional insights. Cell Mol Life Sci, 2019. 76(16): p. 3229-3248.
10. Pardo-Saganta, A., et al., Role of the Extracellular Matrix in Stem Cell Maintenance. Current Stem Cell Reports, 2019. 5(1): p. 1-10.
11. Agrawal, V., et al., Evidence of innervation following extracellular matrix scaffold-mediated remodelling of muscular tissues. J Tissue Eng Regen Med, 2009. 3(8): p. 590-600.
12. Sottile, J., Regulation of angiogenesis by extracellular matrix. Biochim Biophys Acta, 2004. 1654(1): p. 13-22.
13. Marchand, M., et al., Extracellular matrix scaffolding in angiogenesis and capillary homeostasis. Seminars in Cell & Developmental Biology, 2019. 89: p. 147-156.
14. Arenas Gómez, C. M., K. Z. Sabin, and K. Echeverri, Wound healing across the animal kingdom: Crosstalk between the immune system and the extracellular matrix. Developmental Dynamics, 2020. 249(7): p. 834-846.
15. Humphrey, J. D., E. R. Dufresne, and M. A. Schwartz, Mechanotransduction and extracellular matrix homeostasis. Nature Reviews Molecular Cell Biology, 2014. 15(12): p. 802-812.
16. Bissell, M. J., H. G. Hall, and G. Parry, How does the extracellular matrix direct gene expression?J Theor Biol, 1982. 99(1): p. 31-68.
17. Schultz, G. S., et al., Dynamic reciprocity in the wound microenvironment. Wound Repair Regen, 2011. 19(2): p. 134-48.
18. Cragg, B., Brain extracellular space fixed for electron microscopy. Neurosci Letf, 1979. 15(2-3): p. 301-6.
19. Song, I. and A. Dityatev, Crosstalk between glia, extracellular matrix and neurons. Brain Res Bull, 2018. 136: p. 101-108.
20. Fitch, M. T. and J. Silver, Glial cell extracellular matrix: boundaries for axon growth in development and regeneration. Cell Tissue Res, 1997. 290(2): p. 379-84.
21. Dityatev, A. and M. Schachner, Extracellular matrix molecules and synaptic plasticity. Nat Rev Neurosci, 2003. 4(6): p. 456-68.
22. Mouw, J. K., G. Ou, and V. M. Weaver, Extracellular matrix assembly: a multiscale deconstruction. Nat Rev Mol Cell Biol, 2014. 15(12): p. 771-85.
23. Stefanovic, B., RNA protein interactions governing expression of the most abundant protein in human body, type I collagen. Wiley Interdiscip Rev RNA, 2013. 4(5): p. 535-45.
24. Sertie, A. L., et al., Collagen XVIII, containing an endogenous inhibitor of angiogenesis and tumor growth, plays a critical role in the maintenance of retinal structure and in neural tube closure (Knobloch syndrome). Hum Mol Genet, 2000. 9(13): p. 2051-8.
25. Fox, M. A., Novel roles for collagens in wiring the vertebrate nervous system. Curr Opin Cell Biol, 2008. 20(5): p. 508-13.
26. Hubert, T., et al., Collagens in the developing and diseased nervous system. Cell Mol Life Sci, 2009. 66(7): p. 1223-38.
27. Yurchenco, P. D., Basement membranes: cell scaffoldings and signaling platforms. Cold Spring Harb Perspect Biol, 2011. 3(2).
28. Yurchenco, P. D. and J. C. Schittny, Molecular architecture of basement membranes. FASEB J, 1990. 4(6): p. 1577-90.
29. Nguyen, B., G. Bix, and Y. Yao, Basal lamina changes in neurodegenerative disorders. Mol Neurodegener, 2021. 16(1): p. 81.
30. Paulsson, M., Basement membrane proteins: structure, assembly, and cellular interactions. Critical reviews in biochemistry and molecular biology, 1992. 27(1-2): p. 93-127.
31. Yurchenco, P. D. and B. L. Patton, Developmental and pathogenic mechanisms of basement membrane assembly. Curr Pharm Des, 2009. 15(12): p. 1277-94.
32. Martin, G. R. and R. Timpl, Laminin and other basement membrane components. Annu Rev Cell Biol, 1987. 3: p. 57-85.
33. Seppanen, A., et al., Collagen XVII is expressed in human CNS neurons. Matrix Biol, 2006. 25(3): p. 185-8.
34. Seppanen, A., et al., Distribution of collagen XVII in the human brain. Brain Res, 2007. 1158: p. 50-6.
35. Ulbrich, P., et al., Interplay between perivascular and perineuronal extracellular matrix remodelling in neurological and psychiatric diseases. Eur J Neurosci, 2021. 53(12): p. 3811-3830.
36. Zhang, Y., et al., Purification and Characterization of Progenitor and Mature Human Astrocytes Reveals Transcriptional and Functional Differences with Mouse. Neuron, 2016. 89(1): p. 37-53.
37. Fawcett, J. W., T. Oohashi, and T. Pizzorusso, The roles of perineuronal nets and the perinodal extracellular matrix in neuronal function. Nat Rev Neurosci, 2019. 20(8): p. 451-465.
38. Su, J., J. Cole, and M. A. Fox, Loss of Interneuron-Derived Collagen XIX Leads to a Reduction in Perineuronal Nets in the Mammalian Telencephalon. ASN Neuro, 2017. 9(1): p. 1759091416689020.
39. Jovasevic, V., et al., Primary cilia are required for the persistence of memory and stabilization of perineuronal nets. iScience, 2021. 24(6): p. 102617.
40. Hartig, W., et al., Update on Perineuronal Net Staining With Wisteria floribunda Agglutinin (WFA). Front Integr Neurosci, 2022. 16: p. 851988.
41. An, B., Y. S. Lin, and B. Brodsky, Collagen interactions: Drug design and delivery. Adv Drug Deliv Rev, 2016. 97: p. 69-84.
42. Kagan, H. M., Intra- and extracellular enzymes of collagen biosynthesis as biological and chemical targets in the control of fibrosis. Acta Trop, 2000. 77(1): p. 147-52.
43. Ito, S. and K. Nagata, Roles of the endoplasmic reticulum-resident, collagen-specific molecular chaperone Hsp47 in vertebrate cells and human disease. Journal of Biological Chemistry, 2019. 294(6): p. 2133-2141.
44. Kechagia, J. Z., J. Ivaska, and P. Roca-Cusachs, Integrins as biomechanical sensors of the microenvironment. Nature Reviews Molecular Cell Biology, 2019. 20(8): p. 457-473.
45. Kim, G. M., H. Park, and S. Y. Lee, Roles of osteoclast-associated receptor in rheumatoid arthritis and osteoarthritis. Joint Bone Spine, 2022. 89(5): p. 105400.
46. van der Zande, H. J., et al., The mannose receptor: from endocytic receptor and biomarker to regulator of (meta) inflammation. Frontiers in Immunology, 2021. 12: p. 765034.
47. Orgel, J. P. and R. S. Madhurapantula, A structural prospective for collagen receptors such as DDR and their binding of the collagen fibril. Biochimica et Biophysica Acta (BBA)-Molecular Cell Research, 2019. 1866(11): p. 118478.
48. de Almeida, L. G., et al., Matrix metalloproteinases: From molecular mechanisms to physiology, pathophysiology, and pharmacology. Pharmacological Reviews, 2022. 74(3): p. 714-770.
49. Cabral-Pacheco, G. A., et al., The Roles of Matrix Metalloproteinases and Their Inhibitors in Human Diseases. International Journal of Molecular Sciences, 2020. 21(24): p. 9739.
50. Zipfel, P., et al., Matrix Metalloproteinases as New Targets in Alzheimer's Disease: Opportunities and Challenges. Journal of Medicinal Chemistry, 2020. 63(19): p. 10705-10725.
51. Ricard-Blum, S., The collagen family. Cold Spring Harb Perspect Biol, 2011. 3(1): p. a004978.
52. Boraschi-Diaz, I., et al., Collagen Type I as a Ligand for Receptor-Mediated Signaling. Frontiers in Physics, 2017. 5.
53. Nagata, K., Hsp47: a collagen-specific molecular chaperone. Trends in biochemical sciences, 1996. 21(1): p. 23-26.
54. Ichikawa, O., et al., Structural basis of the collagen-binding mode of discoidin domain receptor 2. The EMBO journal, 2007. 26(18): p. 4168-4176.
55. Orgel, J., J. San Antonio, and O. Antipova, Molecular and structural mapping of collagen fibril interactions. Connective tissue research, 2011. 52(1): p. 2-17.
56. Vogel, W. F., R. Abdulhussein, and C. E. Ford, Sensing extracellular matrix: An update on discoidin domain receptor function. Cellular Signalling, 2006. 18(8): p. 1108-1116.
57. Emsley, J., et al., Structural basis of collagen recognition by integrin α2β1. Cell, 2000. 101(1): p. 47-56.
58. Barczyk, M., S. Carracedo, and D. Gullberg, Integrins. Cell and tissue research, 2010. 339: p. 269-280.
59. Mogford, J. E., et al., Vascular smooth muscle avP3 integrin mediates arteriolar vasodilation in response to RGD peptides. Circulation research, 1996. 79(4): p. 821-826.
60. Taubenberger, A. V., et al., The effect of unlocking RGD-motifs in collagen I on pre-osteoblast adhesion and differentiation. Biomaterials, 2010. 31(10): p. 2827-2835.
61. Cai, H., et al., Identification of HSP47 binding site on native collagen and its implications for the development of HSP47 inhibitors. Biomolecules, 2021. 11(7): p. 983.
62. Van Doren, S. R., Matrix metalloproteinase interactions with collagen and elastin. Matrix Biology, 2015. 44: p. 224-231.
63. Siegel, R. C., Biosynthesis of collagen crosslinks: increased activity of purified lysyl oxidase with reconstituted collagen fibrils. Proc Natl Acad Sci USA, 1974. 71(12): p. 4826-30.
64. Cronlund, A. L., B. D. Smith, and H. M. Kagan, Binding of lysyl oxidase to fibrils of type I collagen. Connective tissue research, 1985. 14(2): p. 109-119.
65. Beher, D., et al., Regulation of Amyloid Protein Precursor (APP) Binding to Collagen and Mapping of the Binding Sites on APP and Collagen Type I (*). Journal of biological chemistry, 1996. 271(3): p. 1613-1620.
66. Sela, M. and M. J. Crumpton, The antigens. Volume II/edited by Michael Sela; contributors, Michael J. Crumpton (and seven others). 1974, New York, New York: Academic Press, Inc.
67. Lynn, A. K., I. V. Yannas, and W. Bonfield, Antigenicity and immunogenicity of collagen. J Biomed Mater Res B Appl Biomater, 2004. 71(2): p. 343-54.
68. Furthmayr, H. and R. Timpl, Immunochemistry of collagens and procollagens. Int Rev Connect Tissue Res, 1976. 7: p. 61-99.
69. Heikkinen, A., M. A. Fox, and T. Pihlajaniemi, Collagens as New Players in Nervous System Diseases, in The Collagen Superfamily and Collagenopathies, F. Ruggiero, Editor. 2021, Springer International Publishing: Cham. p. 289-338.
70. Soles, A., et al., Extracellular Matrix Regulation in Physiology and in Brain Disease. Int J Mol Sci, 2023. 24(8).
71. Tian, J., et al., Relationships in Alzheimer's disease between the extent of Abeta deposition in cerebral blood vessel walls, as cerebral amyloid angiopathy, and the amount of cerebrovascular smooth muscle cells and collagen. Neuropathol Appl Neurobiol, 2006. 32(3): p. 332-40.
72. Morrone, C. D., J. Bishay, and J. McLaurin, Potential role of venular amyloid in Alzheimer's disease pathogenesis. International journal of molecular sciences, 2020. 21(6): p. 1985.
73. Downs, M., et al., Matrisome changes in Parkinson's disease. Analytical and Bioanalytical Chemistry, 2022. 414(9): p. 3005-3015.
74. Jin, C.-Y., et al., Study of the collagen type VI alpha 3 (COL6A3) gene in Parkinson's disease. BMC neurology, 2021. 21(1): p. 187.
75. Wallace, D. M., et al., The role of matricellular proteins in glaucoma. Matrix Biology, 2014. 37: p. 174-182.
76. Vithana, E. N., et al., Collagen-related genes influence the glaucoma risk factor, central corneal thickness. Human molecular genetics, 2011. 20(4): p. 649-658.
77. Parmar, A. S., et al., A peptide study of the relationship between the collagen triple-helix and amyloid. Biopolymers, 2012. 97(10): p. 795-806.
78. Forsell, C., et al., Genetic association to the amyloid plaque associated protein gene COL25A1 in Alzheimer's disease. Neurobiol Aging, 2010. 31(3): p. 409-15.
79. Sondag, C. M. and C. K. Combs, Adhesion of monocytes to type I collagen stimulates an APP-dependent proinflammatory signaling response and release of Abeta1-40. J Neuroinflammation, 2010. 7: p. 22.
80. Cheng, J. S., et al., Collagen VI protects neurons against Abeta toxicity. Nat Neurosci, 2009. 12(2): p. 119-21.
81. Wareham, L. K., et al., Solving neurodegeneration: common mechanisms and strategies for new treatments. Mol Neurodegener, 2022. 17(1): p. 23.
82. Lebbink, R. J., et al., Collagens are functional, high affinity ligands for the inhibitory immune receptor LAIR-1. The Journal of experimental medicine, 2006. 203(6): p. 1419-1425.
83. Meyaard, L., LAIR and collagens in immune regulation. Immunology letters, 2010. 1(128): p. 26-28.
84. Xu, J. and G.-P. Shi, Vascular wall extracellular matrix proteins and vascular diseases. Biochimica et Biophysica Acta (BBA)-Molecular Basis of Disease, 2014. 1842(11): p. 2106-2119.
85. Shields, K. J., et al., Complement proteins C3 and C4 bind to collagen and elastin in the vascular wall: a potential role in vascular stiffness and atherosclerosis. Clinical and translational science, 2011. 4(3): p. 146-152.
86. Chen, Y., et al., The complement system in the central nervous system: from neurodevelopment to neurodegeneration. Biomolecules, 2022. 12(2): p. 337.
87. Dudvarski Stankovic, N., et al., Microglia-blood vessel interactions: a double-edged sword in brain pathologies. Acta neuropathologica, 2016. 131(3): p. 347-363.
88. Hong, S., et al., Complement and microglia mediate early synapse loss in Alzheimer mouse models. Science, 2016. 352(6286): p. 712-716.
89. Bradbury, E. J. and E. R. Burnside, Moving beyond the glial scar for spinal cord repair. Nat Commun, 2019. 10(1): p. 3879.
90. Hirano, S., et al., Astrocytes express type VIII collagen during the repair process of brain cold injury. Biochemical and Biophysical Research Communications, 2004. 317(2): p. 437-443.
91. Klapka, N. and H. W. Muller, Collagen matrix in spinal cord injury. Journal of neurotrauma, 2006. 23(3-4): p. 422-436.
92. Bradbury, E. J. and L. M. Carter, Manipulating the glial scar: chondroitinase ABC as a therapy for spinal cord injury. Brain research bulletin, 2011. 84(4-5): p. 306-316.
93. Coleman, C. M., et al., Growth differentiation factor-5 enhances in vitro mesenchymal stromal cell chondrogenesis and hypertrophy. Stem cells and development, 2013. 22(13): p. 1968-1976.
94. Orr, M. B. and J. C. Gensel, Spinal cord injury scarring and inflammation: therapies targeting glial and inflammatory responses. Neurotherapeutics, 2018. 15: p. 541-553.
95. Moeendarbary, E., et al., The soft mechanical signature of glial scars in the central nervous system. Nature communications, 2017. 8(1): p. 14787.
96. Powell, S., M. Irnaten, and C. O'Brien, Glaucoma—‘A Stiff Eye in a Stiff Body’. Curr Eye Res, 2023. 48(2): p. 152-160.
97. Nieuwenhuis, B., et al., Integrins promote axonal regeneration after injury of the nervous system. Biological Reviews, 2018. 93(3): p. 1339-1362.
98. Koopmans, G., B. Hasse, and N. Sinis, Chapter 19: The role of collagen in peripheral nerve repair. Int Rev Neurobiol, 2009. 87: p. 363-79.
99. Chernousov, M. A., et al., Regulation of Schwann cell function by the extracellular matrix. Glia, 2008. 56(14): p. 1498-1507.
100. Wehner, D., et al., Wnt signaling controls pro-regenerative Collagen XII in functional spinal cord regeneration in zebrafish. Nature Communications, 2017. 8(1): p. 126.
101. Bretaud, S., et al., Fishing for collagen function: About development, regeneration and disease. Seminars in Cell & Developmental Biology, 2019. 89: p. 100-108.
102. Mueller, C., et al., Lesional expression of the endogenous angiogenesis inhibitor endostatin/collagen XVIII following traumatic brain injury (TBI). Experimental neurology, 2007. 208(2): p. 228-237.
103. Walia, A., et al., Endostatin's emerging roles in angiogenesis, lymphangiogenesis, disease, and clinical applications. Biochimica et Biophysica Acta (BBA)—General Subjects, 2015. 1850(12): p. 2422-2438.
104. Deininger, M. H., R. Meyermann, and H. J. Schluesener, Endostatin/collagen XVIII accumulates in patients with traumatic brain injury. Journal of neurotrauma, 2006. 23(7): p. 1103-1110.
105. Song, I. and A. Dityatev, Crosstalk between glia, extracellular matrix and neurons. Brain research bulletin, 2018. 136: p. 101-108.
106. George, N. and H. M. Geller, Extracellular matrix and traumatic brain injury. Journal of neuroscience research, 2018. 96(4): p. 573-588.
107. Hapach, L. A., et al., Manipulation of in vitro collagen matrix architecture for scaffolds of improved physiological relevance. Physical Biology, 2015. 12(6): p. 061002.
108. Ren, X., et al., Osteogenesis on nanoparticulate mineralized collagen scaffolds via autogenous activation of the canonical BMP receptor signaling pathway. Biomaterials, 2015. 50: p. 107-114.
109. Estrada, V., A. Tekinay, and H. W. Muller, Chapter 16—Neural ECM mimetics, in Progress in Brain Research, A. Dityatev, B. Wehrle-Haller, and A. Pitkanen, Editors. 2014, Elsevier. p. 391-413.
110. Chen, J.-H., et al., Neuroprotective effects of collagen-glycosaminoglycan matrix implantation following surgical brain injury. Mediators of Inflammation, 2019. 2019.
111. Kadler, K. E., et al., Collagens at a glance. Journal of cell science, 2007. 120(12): p. 1955-1958.
112. Ribeiro, M., et al., Intraocular Delivery of a Collagen Mimetic Peptide Repairs Retinal Ganglion Cell Axons in Chronic and Acute Injury Models. Int J Mol Sci, 2022. 23(6).
113. McGrady, N. R., et al., Restoring the Extracellular Matrix: A Neuroprotective Role for Collagen Mimetic Peptides in Experimental Glaucoma. Front Pharmacol, 2021. 12: p. 764709.
114. Calkins, D. J., Critical pathogenic events underlying progression of neurodegeneration in glaucoma. Prog Retin Eye Res, 2012. 31(6): p. 702-19.
115. Calkins, D. J., Adaptive responses to neurodegenerative stress in glaucoma. Prog Retin Eye Res, 2021: p. 100953.
116. Deister, C., S. Aljabari, and C. E. Schmidt, Effects of collagen 1, fibronectin, laminin and hyaluronic acid concentration in multi-component gels on neurite extension. Journal of Biomaterials Science, Polymer Edition, 2007. 18(8): p. 983-997.
117. Ortega, N. and Z. Werb, New functional roles for non-collagenous domains of basement membrane collagens. Journal of cell science, 2002. 115(22): p. 4201-4214.
118. Chattopadhyay, S., et al., Peptides that anneal to natural collagen in vitro and ex vivo. Org Biomol Chem, 2012. 10(30): p. 5892-7.
119. Chattopadhyay, S., et al., Anchoring a cytoactive factor in a wound bed promotes healing. J Tissue Eng Regen Med, 2016. 10(12): p. 1012-1020.
120. Tang, V. W., Collagen, stiffness, and adhesion: the evolutionary basis of vertebrate mechanobiology. Molecular biology of the cell, 2020. 31(17): p. 1823-1834.
121. Hu, Y., et al., Matrix stiffness changes affect astrocyte phenotype in an in vitro injury model. NPG Asia Materials, 2021. 13(1): p. 35.
122. Meyaard, L., The inhibitory collagen receptor LAIR-1 (CD305). Journal of Leucocyte Biology, 2008. 83(4): p. 799-803.
123. Son, M., Understanding the contextual functions of Clq and LAIR-1 and their applications. Experimental & Molecular Medicine, 2022. 54(5): p. 567-572.
124. Stevens, B., et al., The classical complement cascade mediates CNS synapse elimination. Cell, 2007. 131(6): p. 1164-1178.
125. Quan, W.-Y., et al., Integrin-mediated inhibition of interleukin-8 secretion from human neutrophils by collagen type I. Journal of leukocyte biology, 2010. 87(3): p. 487-491.

### Example 2: Effect of CMPs and CMP-TC Conjugates in Treating Cardiovascular System Disorders

To examine the possible therapeutic effects of CMPs of the invention in treating cardiovascular system disorders, studies were designed to test certain CMPs conjugates in an in vivo setting—the treatment and possible reversal of atherosclerotic plaques in laboratory animals.

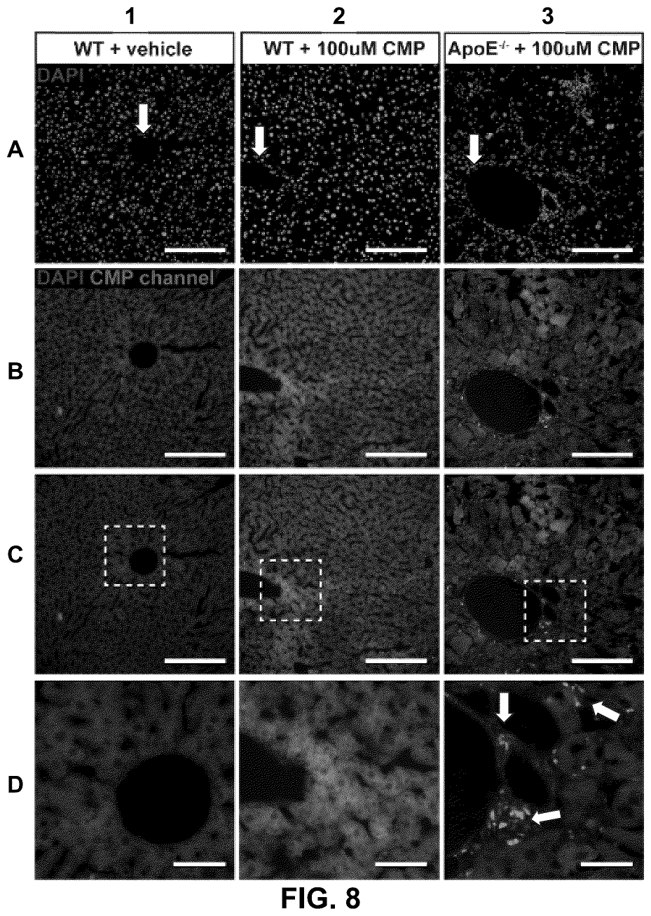

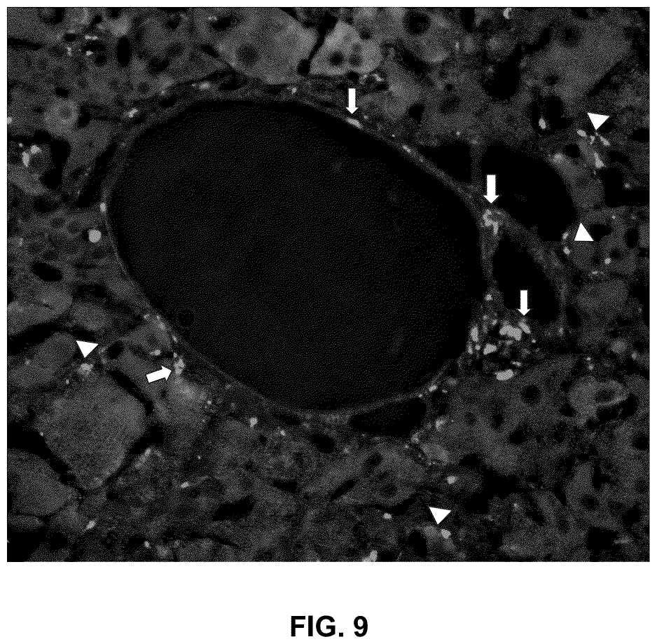

These studies used an in vivo model of diabetes, the ApoE^{-/-} mouse, which is known to develop not only diabetes but accompanying atherosclerotic plaques in its arteries, particularly in the aortic arch. Wildtype or mutant ApoE−/− mice were obtained from Jackson Laboratories (Bar Harbor, ME) and maintained with food and water at their liberty. Mutant mice were treated with streptozotocin (STZ), a compound that has preferential toxicity toward pancreatic R cells and accelerates progression of atherosclerosis (Rerup, CC, Pharmacol. Rev. 22:485-518 (1970); Szkudelski, T et al., Physiol. Res. 50:537-546 (2001); Rosini, A A et al., Proc. Natl. Acad. Sci. USA 74:2485-2489 (1977)), 50 mg/Kg/animal, or with vehicle (PBS) for 5 days. A week after STZ treatment, wildtype and mutant animals were placed on a high-fat diet, and then beginning 7 days thereafter were injected via tail vein with CMP 03A (SEQ ID NO:1) or with PBS three times per week for 10 weeks. Following this treatment, mice were injected twice more (48 hours apart) with vehicle or with fluorescently tagged CMP 03A (100 μl of a 100 μM solution) via tail vein, and sacrificed 48 hours after the second injection. Animals were perfused with fixative and the liver from each mouse was cryosectioned at 10 μm. Perfused liver slices were mounted with DAPI mounting solution (to visualize cell nuclei) and imaged at 20× using 405 nm (for DAPI) and 488 nm (for fluorotagged CMP) channels. Results of these studies are shown in FIG. 8. DAPI staining (FIG. 8, A1-A3) showed the presence of intact liver cells with prominent vessel lumens (arrows) distributed throughout. Simultaneous evaluation of DAPI and fluorescent CMP (FIG. 8, B1-B3) demonstrated diffuse CMP fluorescence in the wildtype mouse (FIG. 8, B2), mostly localized around the vessel lumen (where the collagen-containing basement membrane is continuously undergoing turnover), while the sections from the ApoE^{-/-} mutant mouse showed substantial punctate CMP fluorescence not only around the vessel but also through the liver parenchyma (FIG. 8, B3), indicating that there was significant collagen damage within the liver tissue itself. These results were confirmed when CMP fluorescence alone was viewed in these same sections (FIG. 8, C2, C3). Indeed, high power views of the sections inset from FIG. 8 C2-C3 showed localization of the CMPs in the areas of expected liver damage associated with the atherosclerosis model (FIG. 8, D3, arrows), in a way not seen in liver sections from wildtype mice (FIG. 8, D2). Further examination of the inset section from the mutant mouse at even higher magnification (FIG. 9) confirmed this localization of CMP, indicating collagen damage, not only in the vessel basement membrane (arrows) but also within the parenchyma of the liver (arrowheads).

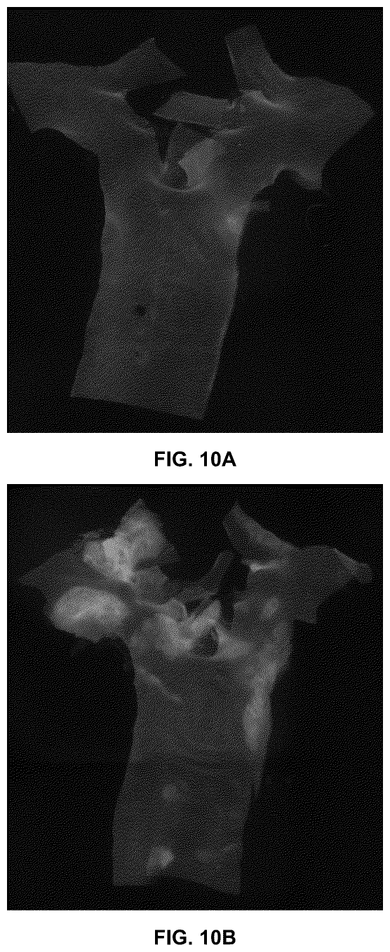

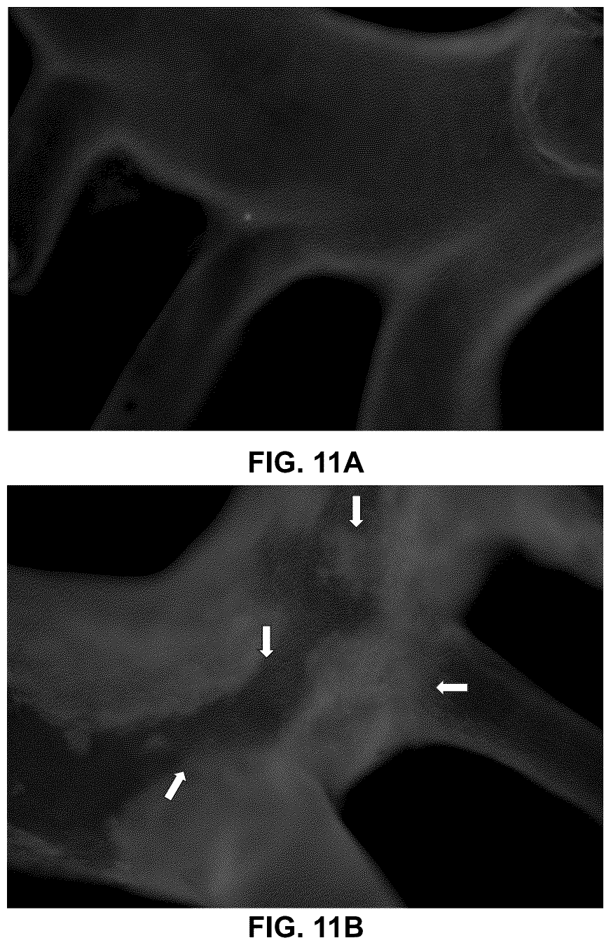

These results in mouse liver were confirmed when the aortic arches from wildtype and ApoE^{-/-} mutant mice were dissected and examined for localization of fluorescent CMP. As shown in FIG. 10, the aortic arch from wildtype mice showed only diffuse staining, indicating that CMP was binding to collagen in the tissue that was probably undergoing normal turnover, leading to a diffuse and faint staining pattern (FIG. 10A). In contract, the aortic arch dissected from ApoE^{-/-} mice (FIG. 10B) demonstrated substantial and differentiated localization of CMP in the vessel walls in areas of typical plaque accumulation in atherosclerotic animals. This pattern was confirmed in additional sections (FIG. 11), which demonstrated not only the increased accumulation of CMP in presumably atherosclerotic areas of the aortic arch in mutant mice (FIG. 11B) in a way not seen in wildtype mice (FIG. 11A), but also an accumulation of CMP—indicative of collagen damage—in areas of the vessel where vasoocclusion was apparent (FIG. 11B, arrows).

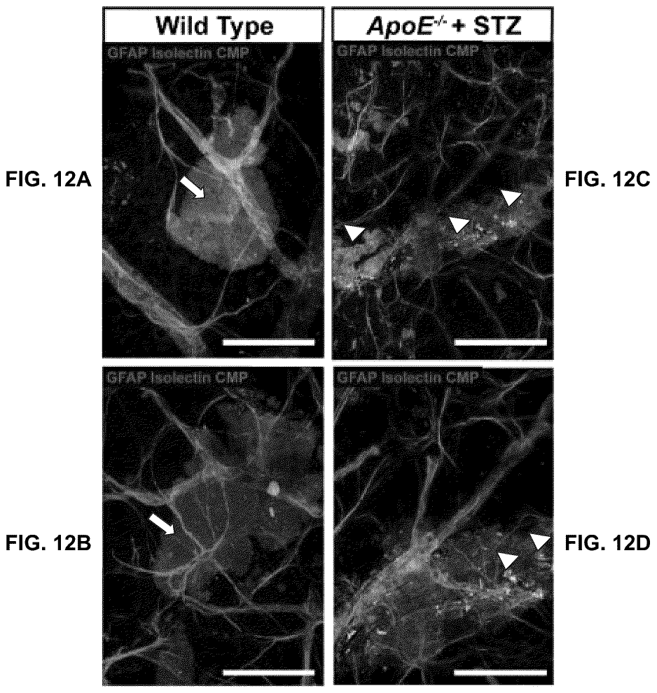

Finally, the retinal vasculature and nerve processes in wildtype and ApoE^{-/-} mice were examined for CMP localization with respect to blood vessels and glial astrocytes in these areas. Results are shown in FIG. 12. In wildtype mice (FIG. 12A, 12B), CMPs were diffusely located throughout the retinal tissue (arrows), in areas near the retinal blood vessels, typical of normal ECM turnover and endogenous remodeling as described hereinabove. In contrast, sections from ApoE−/− STZ-treated mice showed substantial accumulation of CMP in localized areas of the retinal blood vessels (FIGS. 12C, 12D, arrowheads), particularly in those areas showing substantial glial astrocyte infiltration presumably due to inflammation in those areas. Thus, in addition to affecting the liver tissue and vasculature, diabetes and accompanying atherosclerosis in this mouse model clearly caused accumulation of CMPs in areas surrounding the retina, indicating damage to the retinal collagen in the area of the optic nerve.

These results indicate that damaged collagen accumulates in areas of plaque development and other tissues (aorta, retina) in a mouse model of atherosclerosis. These findings are consistent with those reported by others, indicating that collagen damage is a sign of early atherosclerosis development (Smith, K A et al., J. Cardiovas. Transl. Res. 16:463 (2023)). Given that, as amply described herein, CMPs are known to repair disrupted collagen in a variety of tissues in humans and other mammals, these results suggest that CMPs have promise as a potential therapeutic in treating, and potentially reversing, atherosclerosis by repairing the collagen damage that appears to underly the development and progression of atherosclerotic plaques. CMPs also appear to localize in the retinas of atherosclerotic mice in a highly localized way, indicating that they may be useful in treating and preventing certain ocular diseases that are associated with atherosclerosis and diabetes, such as diabetic retinopathy. Moreover, given that certain central nervous system disorders (e.g., dementia) share some risk factors with atherosclerosis (Nordestgaard, L T et al., Int. J. Mol. Sci. 23:9777 (2022)), CMP treatment may be useful in simultaneous treatment of nervous system disorders and diseases as well as cardiovascular system disorders and diseases.

### Example 3: Effect of CMPs and CMP-TC Conjugates in Treating Vascular Disorders in the Central Nervous System

Neurodegenerative diseases and disorders of the central nervous system (CNS) are often associated with cerebrovascular dysfunction. Compromised blood flow to the brain occurring as a result of vascular remodeling, often due to conditions such as hypertension and atherosclerosis, accelerates cognitive decline and the progression of neurodegenerative diseases such as Alzheimer's disease and dementia (1). Vascular remodeling is an adaptive process that occurs in response to both physiological and pathophysiological changes in the vascular microenvironment (2). The vascular microenvironment comprises a variety of extracellular matrix (ECM) proteins and certain ECM-degradative proteases, which act in concert to regulate cellular physiological and pathophysiological processes (2). Integral to the ECM are collagenous proteins which provide structure and stability to a wide range of tissues, including the vasculature (3). Collagens I and III are most abundant in the vascular wall, while collagen IV is the main component of the basement membrane—an integral part of the blood-brain-barrier. Endothelial cells (ECs) line the inner surface of vessels and serve as the barrier between the blood and vascular wall. ECs become dysfunctional in early vascular disease, instigating large-scale vascular remodeling that sometimes precedes eventual neurodegeneration in the CNS. Vascular remodeling also characterizes atherosclerosis, the most common cause of cardiovascular disease worldwide (4). Remodeling in atherosclerosis is associated with disruptions in collagen within the ECM (5). In the late stages of atherosclerosis, vascular cells including ECs secrete increased levels of matrix metalloproteases (MMPs), resulting in the proteolytic cleavage of collagen, and destabilization of atherosclerotic plaques, thereby risking vessel rupture and thrombus formation (6). Finally, patients with diabetes mellitus often exhibit accelerated atherosclerosis progression due to early EC damage and dysfunction (7, 8).

The results of the preceding Examples, and other work published by the present inventors (3, 9, 10), have highlighted the potential use of collagen mimetic peptides (CMPs) to repair collagen and counteract certain neurodegenerative processes. As described hereinabove, CMPs are known to interact with and anneal to damaged collagen to restore the helical structure of this important ECM component (11-13). In the present Example, the inventors conducted a study to explore the use of a CMP in preserving vascular architecture in systemic cardiovascular disease using an established model of atherosclerosis that uses streptozocin (STZ) to accelerate EC dysfunction in ApoE−/− (apolipoprotein E-deficient) mice maintained on a high-fat diet (“HFD”) (14, 15). As noted in Example 2, this mouse strain is the most widely used for preclinical studies of atherosclerosis. Deficiency in endogenous ApoE in these mice leads to cytokine and protease secretion with subsequent inflammation and extracellular matrix degradation (16, 17), while systemic STZ application induces a form of type I diabetes through reduced endothelium-dependent vasodilator response and destruction of pancreatic islet β-cells (18,19).

### Materials and Methods

Animals. All animal studies were conducted in accordance with the NIH guide for the care and use of laboratory animals and approved by the Vanderbilt University Institutional Animal Care and Use Committee. For the studies described, 12-week-old ApoE^{-/-} (#002052) or C57/B6J wildtype (WT) (#000664) mice were obtained from Jackson Laboratories. Mice were housed in a facility managed by Vanderbilt University Division of Animal Care with ad libitum access to water and standard diet followed by ad libitum access to 45% high fat diet (Research Diet Inc.; Catalog #D12451) starting at the correct study time point. Mice were subjected to 12-hr. light/dark cycle.

Animal Model of Diabetic Atherosclerosis. A diabetic atherosclerosis mouse model was implemented by combining ApoE^{-/-} mice with a streptozocin STZ-induced diabetic phenotype. Prior to STZ injection and diet change, baseline fasting blood glucose measurements and weights were obtained. To induce hyperglycemia ApoE^{-/-} mice were injected with STZ (50 mg/Kg in 10 mM sodium citrate, pH4.5; Millipore Sigma Catalog #S0130) intraperitoneally for five consecutive days. For the duration of STZ injections, mice received drinking water supplemented with 10% sucrose. One week after the final STZ injection, blood glucose levels were measured to confirm hyperglycemia. Mice were fasted for 6 hours prior to glucose measurement and then anesthetized with 2.5% isoflurane. Tail snips were taken, and one drop of blood was placed on test strips to obtain readings. Tails were then stemmed of bleeding using a silver nitrate applicator (Avoca reference number #7482) before returning mice to their cages. Any mice not registering as hyperglycemic were removed from the study. Remaining mice were then switched to a 45% high fat diet (HFD) to accelerate atherosclerotic plaque formation. After 1 week on HFD, mice were randomly assigned to receive intravenous CMP (n=5) or PBS (1×PBS, n=4) once weekly for 12 weeks. Upon conclusion of the study, mice were anesthetized via intraperitoneal pentobarbital injection followed by transcardial perfusion of 1×PBS and 4% paraformaldehyde (PFA). One day prior to sacrifice, mice were injected via tail vein injection with Sulfo-NHS-Biotin in sterile 1×PBS (Thermo-Fisher; Catalog #21335).

Intravenous Administration of CMPs. The CMP used in the present Example was a 21-aa single-strand peptide consisting of a 7-repeat sequence of a tripeptide of 4-fluoroproline (Flp), hydroxyproline (Hyp), and glycine (Gly), and abbreviated as (cis-Flp-Hyp-Gly)₇ (SEQ ID NO:6). This CMP was manufactured using standard solid-phase peptide synthesis chemistry in limited quantity by Bachem, AG (Germany). CMP was dissolved in sterile 1×PBS at a concentration of 1 mg/Kg and sterilized using a 0.22 m filter (Millipore Sigma, Burlington, MA, USA). Mice were weighed and restrained in an apparatus designed for tail vein injection. Tails were heated to dilate the veins prior to weekly injection, and 100-150 L of CMP in sterile 1×PBS (vehicle) was injected to accommodate a final concentration in each animal of 0.1 mg/Kg. An additional batch of this CMP was produced with attachment of the TideFluor™ 2 moiety (AAT Bioquest, Sunnyvale, CA, United States) to the peptide as a fluorescent reporter. This labeled CMP was injected intravenously at 100 μM concentration (100 μL) to identify binding in WT vs. ApoE^{-/-}+STZ mice. CMP was injected 48 hours prior to sacrifice and again immediately before transcardial perfusion of 4% PFA. One day prior to euthanasia, tail veins were injected with Sulfo-NHS biotin to track vascular leakage. Sulfo-NHS Biotin (Thermo-Fisher; Catalog #21335) was dissolved in sterile 1×PBS (20 mg/mL) and sterilized through a 22 μm filter. Mice were then restrained in tail vein apparatus and injected intravenously with 100 μL Sulfo-NHS biotin. Organs of interest (spleen, kidney, lung, liver, heart, retina, and brain) were cryosectioned at a thickness of 10 μm and mounted on microscope slides with DAPI Fluoromount-G counter stain (Catalog #0100-20; Southern Biotech, Birmingham, AL, USA) for imaging.

Immunohistochemistry and Fluorescent Imaging. Perfused brains were removed from 4% PFA after 24 hours and cryoprotected in a sucrose gradient series (20% to 30%). Brains were cut into 50 μm sections using a freezing sliding microtome (SM2000R; Leica Biosystems, Buffalo Grove, IL, USA), and slices containing hippocampus were identified for immunolabeling. Tissue was cryopreserved by sucrose gradient and underwent 3 freeze-thaw cycles for permeabilization. Sections were washed for 10 minutes in 1×PBS and further permeabilized in 0.5% Triton X-100 in 1×PBS overnight at 4° C. Sections were blocked at room temperature (5% normal donkey serum in 0.1% Triton X-100 in 1×PBS) for 2 hours while shaking, followed by primary antibody incubation. Antibodies against CD31(rat 1:200; BD Pharmingen, 550274), collagen IV (goat 1:100, EMD Millipore AB769), and GFAP (goat 1:200; Abcam, Cambridge, UK, Catalog #ab53554) or isolectin GS-IB4 biotin-XX conjugate (1:200; Invitrogen; Waltham, MA, USA, Catalog #121414) in 3% normal donkey serum in 1×PBS and 0.1% triton were incubated at 4 C for 3-5 days while rocking. Sections were washed with 1×PBS and placed in secondary antibody solution (1% donkey serum and 0.1% Triton in 1×PBS) containing secondary antibodies: donkey anti-rat Alexa 488 (1:200; Jackson Immuno Research; 712-545-150), donkey anti-goat Alexa 555 (1:200; Jackson Immuno Research; Catalog #705-565-147), and Strepdavadin Alexa-647 (1:200; Invitrogen; Catalog #S21374). The Strepdavadin binds to Sulfo-NHS Biotin for fluorescent visualization of injected biotin. Sections were incubated for 2 hr at room temperature before washing in 1×PBS. Sections were mounted on microscope slides (Diamond White Glass Microscope Slides; White frosted; #1358W) with DAPI Fluoromount-G (Catalog #0100-20, SouthernBiotech, Birmingham, AL, USA) and coverslipped (microscope cover glass; Globe; #1414-0) sealed with nail polish (Ted Pella Inc; #114-7) for imaging. Fluorescent images of the retina and brain were taken on Nikon Ti-E Spinning Disk confocal microscope. 10× montages were taken of the brain to orient to the hippocampal CA1 region, dentate gyrus (DG), and cerebral cortex, and closer, region-specific images were then acquired at 20× or 60× magnification. Z-stacks of equal thickness were taken at a step size of 0.3 μm. Z-stacked images were combined using the standard deviation Z-stack option in Fiji ImageJ. For all other tissues (lung, kidneys, liver, spleen, and heart) 20× and 10× magnification were used on the Nikon Ni Eclipse fluorescence microscope.

Quantification and Statistical Analysis. Mean fluorescent intensities of GFAP, CD31, collagen IV, and Sulfo-NHS biotin immunofluorescence in the hippocampus CA1 region, dentate gyrus (DG), and cerebral cortex were obtained using the measure tool in Fiji software. CD31-stained images were thresholded and binarized. The ImageJ ‘Particle Analysis’ 30-infinity tool was then used to find blood vessels and capture shape descriptor information including area, perimeter, major and minor axes, and Feret diameter. The distance from astrocytes to vessels was measured using GFAP-stained images of CA1 and DG brain regions. Cortex was not included in the analysis due to lack of uniform astrocyte density. The ‘Particle Analysis’ 30-infinity tool was used to find individual astrocytes bodies and their centroid was measured. Using a custom Python script, the minimum distance from each centroid to the closest blood vessel was recorded. All quantification was performed on multiple labeled sections through each brain (typically, 2-3 per animal); 12 confocal images were taken from each brain at 60× magnification, four each for cortex, CA1, and DG. Statistics were conducted in GraphPad Prism version 10.1.2. After testing for normality, ordinary one-way ANOVA, Brown-Forsythe one-way ANOVA, and nonparametric tests including Kruskal Wallis and Mann Whitney were used.

### Results

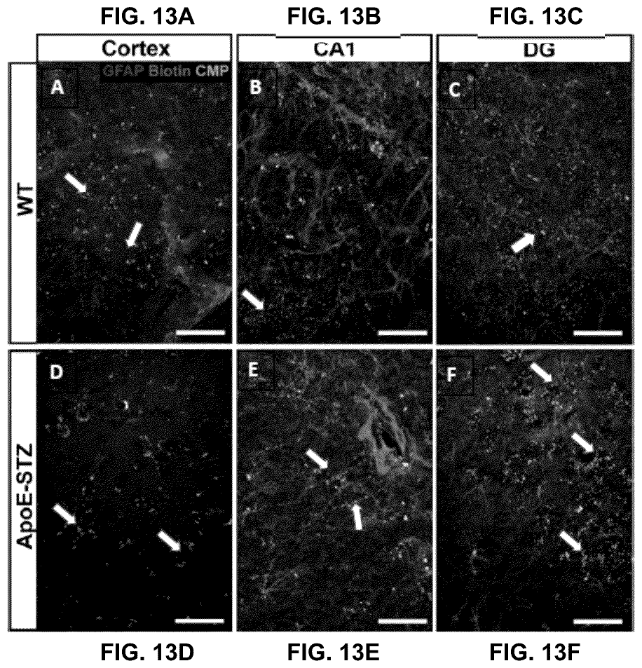

1. Intravenously Injected CMP Crosses the BBB. To determine whether CMP crosses the blood-brain-barrier, we intravenously injected healthy WT and diseased ApoE-STZ mice with fluorescent-labelled CMP. CMP binding, demonstrated by regions of punctate fluorescent CMP localizing to areas of damaged collagen, was detected in the cerebral cortex, hippocampus CA1 region and dentate gyrus (DG) regions of the brains of both WT and ApoE-STZ mice (FIG. 13; arrows). The pattern of CMP binding in the brains of ApoE-STZ mice (FIG. 13D-13F), however, was distinct and more organized than the uniform binding observed in the WT animals (FIG. 13A-13C). These results indicated that intravenously injected CMP crosses the blood-brain barrier, and localizes to areas of disorganized collagen which are elevated in animals with diabetic atherosclerosis.

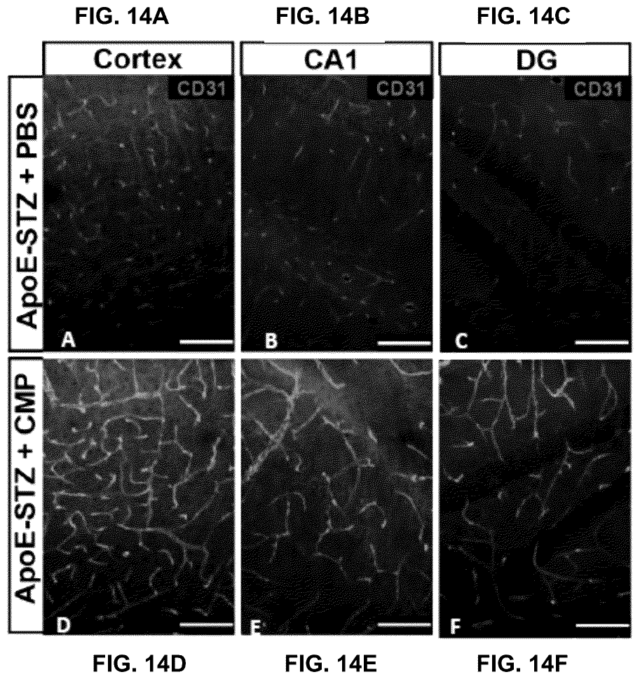

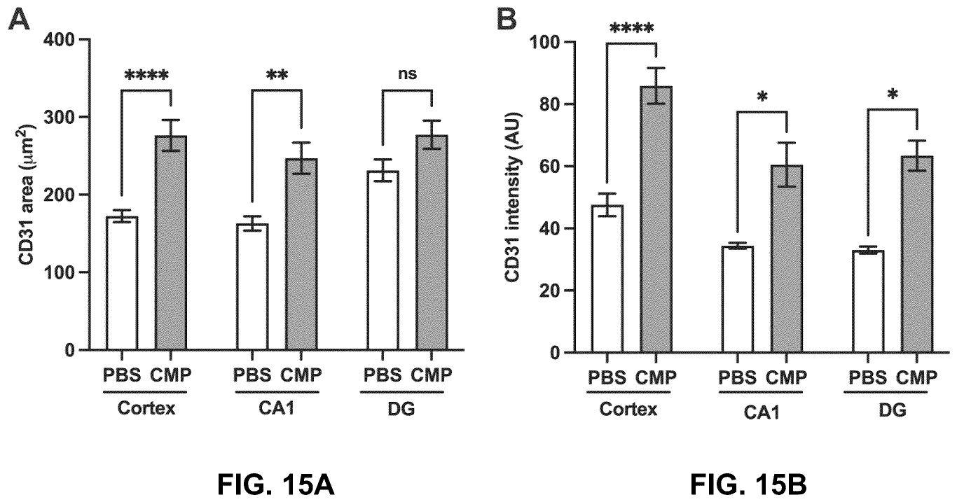

2. CMP Increases Endothelial CD31 and Collagen IV Expression, and Helps Preserve Cerebrovascular Architecture. To assess vascular structure and EC density, brain sections were immunolabeled with antibodies binding to PECAM-1 (CD31), an effective EC and vascular marker. As shown in FIG. 14, CD31 labeling was far more pronounced in CMP-treated animals (FIG. 14D-14F), compared to PBS-treated controls (FIG. 14A-14C), in all brain regions. Upon quantification of the area and intensity of CD31 staining in the various brain regions in PBS-treated vs. CMP-treated mice, there was a significant increase in the area of CD31-positive vessels in CMP-treated mice compared to those treated with PBS in both the CA1 and cortex (FIG. 15A; p=0.001 and p<0.0001, respectively). Change in CD31 area was region-specific; for example, there was no significant difference in vessel area in the DG between CMP-treated animals and control. Interestingly, CD31 intensity was increased in CMP-treated mice compared to PBS-treated controls in all three brain regions of interest (FIG. 15B): hippocampus (CA1), cortex, and dentate gyrus (DG) (p=0.02, p<0.0001, and p=0.01, respectively). These results indicated that CMP treatment enhances the organization of ECs and the formation (or maintenance of structural integrity) of blood vessels in the brains of animals with diabetic atherosclerosis.

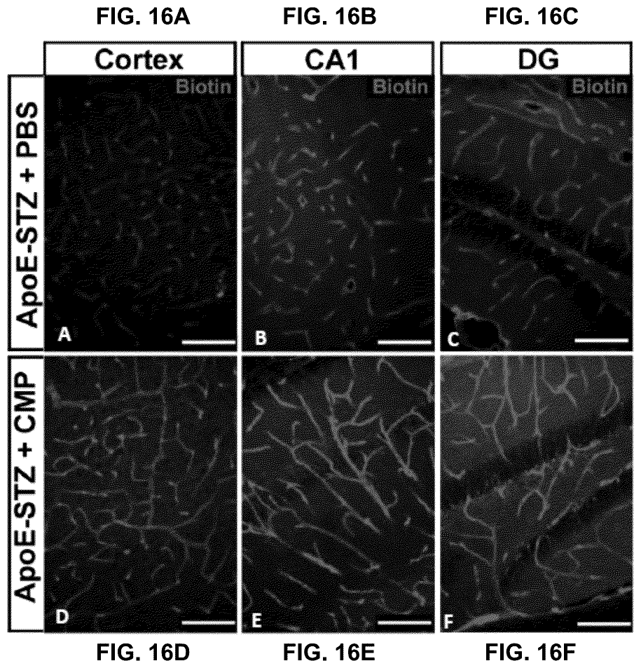

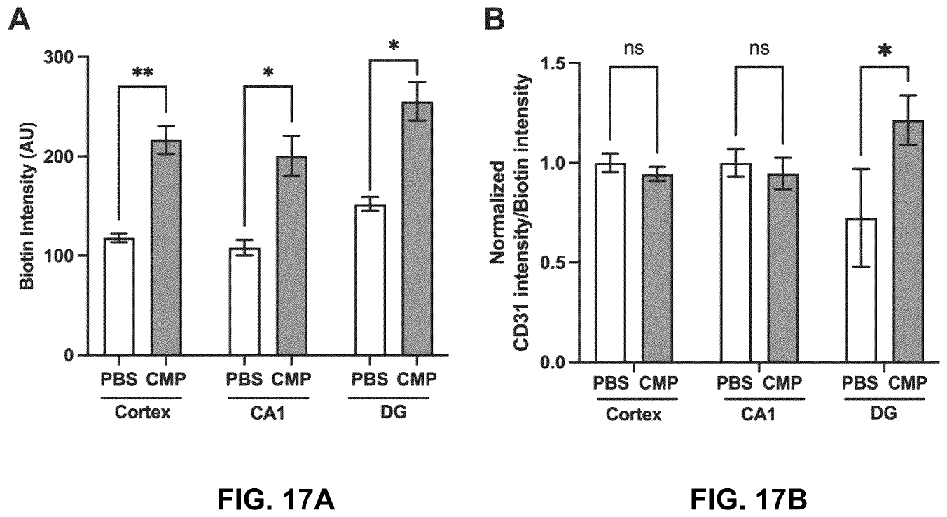

To further elucidate the impact of CMP treatment on cerebrovascular integrity, we next assessed vascular leakage and vessel structure in these animals. To conduct these studies, biotin was injected intravenously into PBS- and CMP-treated and then detected using a fluorescently labeled streptavidin. Representative images of biotin labeling in the CA1 cortex, and DG are shown in FIG. 16. At this timepoint (3 months post-treatment), vascular leakage of biotin was not observed across the vasculature of either PBS- or CMP-treated mice. However, biotin labeling highlighted differences in vascular structure between these groups. Specifically, in PBS-treated mice (FIG. 16A-16C), vessels were shorter and more fragmented in all brain regions, whereas CMP-treated mice (FIG. 16D-16F) exhibited longer, more complex vessels with increased branching. When biotin intensity was quantified as a secondary measure of vessel density, CMP-treated animals had increased biotin labeling in the CA1, DG, and cortex regions compared to PBS-treated controls (FIG. 17A). Finally, we evaluated whether an increase in CD31 intensity observed in FIG. 17A may have been due to increased vessel density, by expressing CD31 intensity as a ratio of biotin intensity (FIG. 17B). Interestingly, CMP treatment only significantly increased vessel CD31 intensity in the DG (compared to PBS treatment) when biotin intensity was taken into account, indicating that the increased biotin intensity observed in most brain regions of CMP-treated animals vs. PBS-treated controls (except DG) was due not necessarily to an increase in the number of vessels but more likely to an increased structural integrity in the vessels that were present in CMP-treated animals relative to vehicle-treated controls.

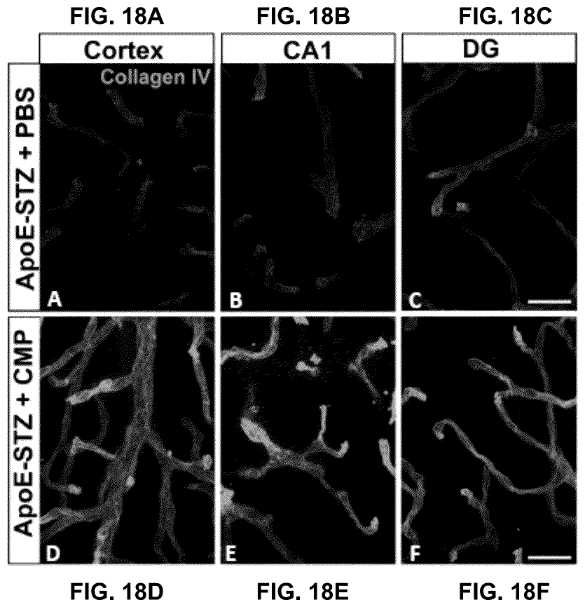

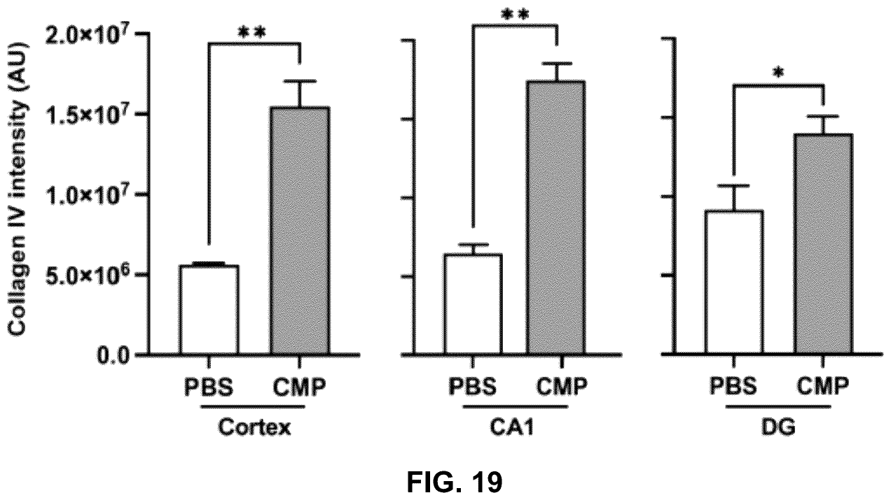

We next wanted to assess the health of the vasculature in the brains of CMP-treated ApoE-STZ mice vs. negative controls, by determining the levels of expression of type IV collagen in the brains of these animals. As noted above, type IV collagen is enriched in the basement membranes of intact blood vessels in all regions of the body, including in the cerebrovasculature. Therefore, to assess the impact of CMP treatment on vascular collagen IV levels in the brains of ApoE-STZ mice, we labelled brain sections from animals treated with PBS vehicle or with CMP with an antibody to collagen IV (FIG. 18). Collagen IV intensity in vehicle-treated animals (FIG. 18A-18C) was observed to be low in the cortex, CA1, and DG. In the CMP-treated group (FIG. 18D-18F), however, collagen IV intensity was higher and vessel structure better defined in all brain regions assessed. As shown in FIG. 19, relative to PBS-treated negative controls, CMP treatment increased collagen IV intensity by 175% (p=0.01) in cortex (FIG. 18A vs. 18D), by 170% (p=0.01) in CA1 (FIG. 18B vs. 18E), and by 52% (p=0.04) in DG (FIG. 18C vs. 18F) brain regions.

Taken together, these results provide compelling evidence supporting the conclusion that that CMP treatment enhances the organization of ECs and the formation (or maintenance of structural integrity) of blood vessels in the brains of animals with diabetic atherosclerosis.

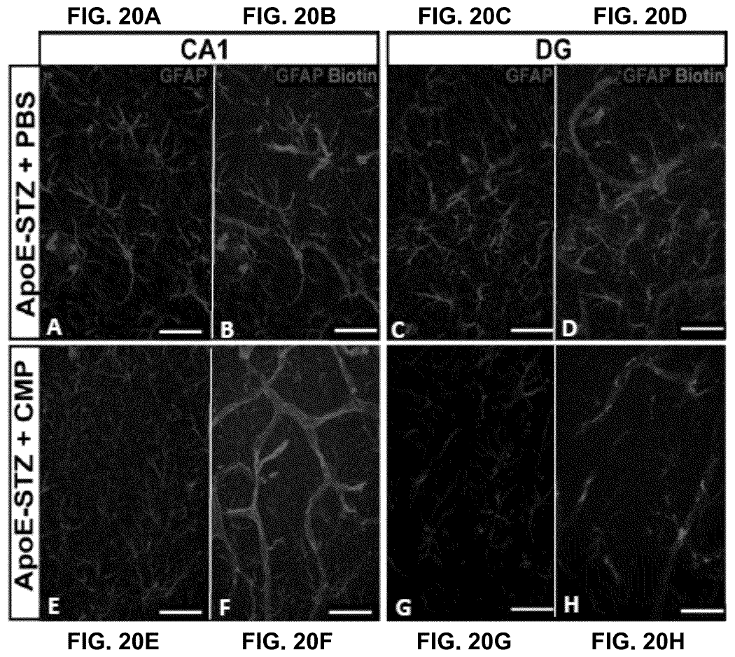

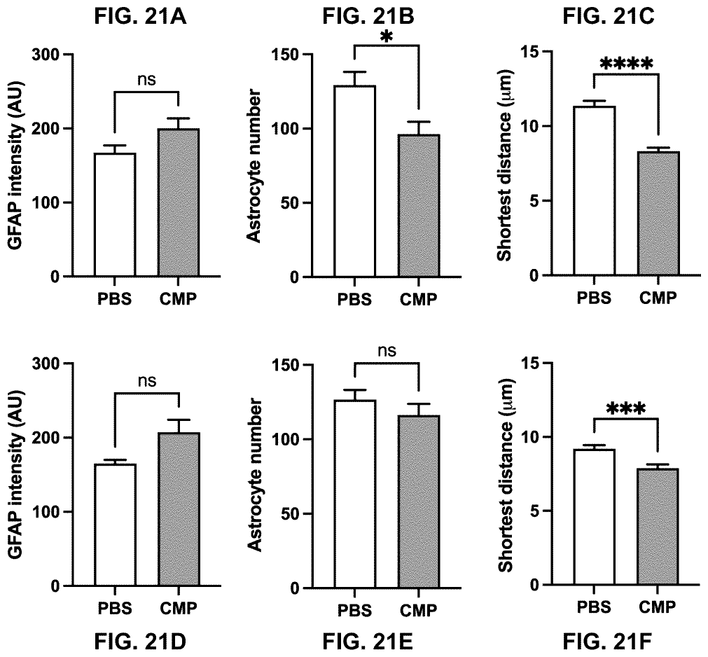

3. CMP Treatment Promotes Astrocyte-Vascular Interactions in the Brain. To evaluate the impact of CMP treatment on glial-vascular interactions in the CA1 and DG brain regions, we quantified astrocyte density and number, and their proximity to blood vessels, in CMP- and PBS-treated brains of ApoE-STZ mice, using fluorescently labeled anti-GFAP antibodies. The cortex was excluded from evaluation due to lack of GFAP staining (see FIG. 13A, 13D). Representative images of astrocytes (FIGS. 20A, 20C, 20E and 20G) and their vessel interactions (FIGS. 20B, 20D, 20F and 20H) are shown in FIG. 20 for PBS- and CMP-treated animals. Upon quantification of staining intensity, GFAP density was not significantly different in CMP-treated mice compared to PBS in both the CA1 (FIG. 21A; p=0.07) and DG (FIG. 21D; p=0.06) regions. Next, we identified and enumerated individual astrocyte cell soma in representative microscopic fields. The number of astrocyte soma decreased in the CA1 region of CMP-treated mice compared to PBS controls (FIG. 21B; p=0.02), while the number of astrocyte soma in the DG was not significantly different between CMP-treated and control groups (FIG. 21E; p=0.31). Finally, using astrocyte soma coordinates, we calculated the distance from individual astrocytes to the nearest blood vessel in brain sections in CMP- and PBS-treated mice. Interestingly, despite a reduction in the number of astrocytes in the CA1, in the CMP-treated group astrocytes were closer to blood vessels than in PBS-treated mice in both the CA1 (FIG. 21C; p<0.0001) and DG regions (FIG. 21F; p=0.0004). These results indicate that CMP treatment promotes increased interactions between accessory astrocytes and blood vessels in the brains of animals with diabetic atherosclerosis.

### Discussion

In our previous work, we demonstrated the potential of CMP repair of collagen to counteract neurodegeneration in the optic nerve projection to the brain (3, 9, 10). Diabetes and atherosclerosis are systemic diseases associated with age-related neurodegenerations of the CNS including Alzheimer's disease, diabetic retinopathy, and dementia21-24. Since there is an emergent role of vascular dysfunction as an early driver of neurodegeneration25, we explored whether CMPs have the potential to preserve the cerebrovasculature in a mouse model of accelerated diabetic atherosclerotic disease (ApoE^{-/-} STZ mice fed a HFD) (26, 27, 28).

Mice with deficiency of ApoE fed a normal diet exhibit an imbalance of cholesterol in macrophages, causing high levels of cytokine and protease secretion, and triggering inflammation and ECM degradation (17). Feeding ApoE−/− mice a high fat diet leads to an acceleration of atherosclerotic plaque formation and widespread inflammation, with increased adhesion molecule expression and leukocyte recruitment, as well as degeneration of the microvessels and a reduction in microvascular length (16). Changes in vessel structure correlated with activation of the proinflammatory cyclophilin A (CypA)-nuclear factor-KB-matrix metalloproteinase-9 (MMP9) pathway (29). Activation of MMP9 leads to the degradation of collagen in the ECM, which may promote inflammation and vessel degradation (30). STZ is an antibiotic that causes pancreatic islet B-cell destruction, triggering hyperglycemia (19). When STZ is combined with ApoE deficiency, mice exhibit gross endothelial cell dysfunction (18).

To investigate the effect of CMP treatment on ApoE-STZ pathphysiology (as a model of diabetic cerebrovascular pathology), we treated mice with intravenous PBS or CMP for a period of 12 weeks and assessed vascular structures in the brain. All mice included in the study became hyperglycemic after STZ treatment compared to baseline measurements. CMP crossed the blood-brain barrier (FIG. 13) and had a positive effect on brain vasculature. Compared to PBS-treated animals, CMP treatment elevated the expression of CD31 in the cortex, CA1, and DG (FIGS. 14 and 15). Furthermore, vascular area (assessed by CD31 area and biotin intensity) was also increased in CMP-treated mice relative to negative controls (FIGS. 15-17). After normalizing vs. biotin area, CMP treatment increased CD31 to the highest extent in the DG (FIGS. 18, 19). Analogously, CMP treatment was seen to elevate the expression of collagen IV in all brain regions (FIG. 20). CMP also promoted astrocyte-blood vessel interactions; astrocyte soma were closer in proximity to vessels after CMP treatment when compared to PBS treatment (FIG. 21).

Injection of a fluorescent CMP confirmed that the mimetic peptide crossed the blood-brain barrier, reaching the cortex and hippocampus of the brain. Interestingly, binding of CMP was evident in both healthy WT and diseased ApoE^{-/-} animals, suggesting that degraded collagen was present in the tissues of both phenotypes. This observation is not surprising since baseline collagen turnover within the ECM and vascular basal lamina occurs in healthy tissues as well as in disease during homeostasis processes such as tissue remodeling. ECM turnover is a dynamic but tightly controlled process; in homeostasis a balance between protein degradation and formation exists (3, 37, 38). In disease however, the balance is disrupted with increased expression of MMPs, leading to increased production of fragmented and degraded collagen with exposed ligand binding sites that may be involved in multiple pro-inflammatory pathways (3).

When assessing the effect of CMP on vasculature in the brain, we first quantified levels of the EC and vascular marker, CD31 (39). CD31, or Platelet/Endothelial Cell Adhesion Molecule-1 (PECAM-1), is a cell adhesion molecule highly expressed on the surface of ECs, and to a lesser extent by a range of immune cells including monocytes, neutrophils, and certain T-cell subsets. CD31 is an integral component of the microvascular barrier, forming tight junctions at EC borders that mediate vascular permeability and leukocyte trafficking (39). In the cortex and hippocampal areas of the brain that we explored, CD31 expression was elevated by CMP treatment relative to negative controls; however, upregulation was most prominent in the vasculature of the DG. Furthermore, in vehicle-treated animals collagen IV levels were highest in the DG, compared to the cortex and CA1 where collagen IV levels were much lower. In vascular dementia, ischemic injury is known to affect brain areas differently. For example, in contrast to other brain regions, the DG has compensatory mechanisms that occur in response to ischemia, e.g., synaptic plasticity, activation of resident glial cells, neovascularization, and proliferation of stem cells (40). In Alzheimer's disease patients, the DG also exhibits resistance to the accumulation of plaques (41). The fact that the DG is able to undergo neurogenesis (42) and exhibits the highest levels of CD31 in CMP-treated animals suggests that helical collagen in the DG has an integral role in EC physiology, which may in turn impact neuronal health and neurogenesis. Given the promising results observed here, collagen mimetic peptides likely are important therapeutic compounds for use in promoting neurogenesis and cognitive function in humans and veterinary animals.

At the endpoint in this study (12 weeks), we did not detect overt vascular leakage using intravenous biotin injection. We did however detect a decrease in vessel length and remodeling with disease that appeared to be prevented with CMP treatment. Since CMP binds to and preserves collagen native structure, it is not surprising that vessel architecture was preserved. CMP also increased CD31 expression on ECs which is necessary for maintaining immune privilege of vascular endothelium and for preserving vascular structure and integrity (44,45). Thus, the improved vascular structure we observed may also be due to elevation of CD31 induced by CMP treatment, which again suggests a potential therapeutic benefit of CMP treatment in such contexts since preventing vascular breakdown is crucial in maintaining the blood-brain barrier and immune privilege of the brain to prevent neurodegeneration (46). Finally, our results suggest that CMP treatment promotes vascular-astrocyte interaction at the cellular level. Astrocytes are glial cells that together with endothelial cells, pericytes, and microglia maintain a tight blood-brain barrier through the formation of a neurovascular unit (47). Our results suggest that CMP treatment will be beneficial in preventing neurovascular breakdown and even promoting cerebrovascular nerve regeneration and growth, as well as improvements in the integrity of the cerebrovascular network itself, which clearly have therapeutic implications for treating and/or preventing CNS diseases including Alzheimer's Disease and other dementia disorders, as well as PNS diseases such as amyotrophic lateral sclerosis, multiple sclerosis, Parkinson's Disease and Huntington's Disease, as well as diseases and disorders characterized by blockage, trauma or leakage of the brain vasculature such as stroke, aneurysm, brain hemorrhages, brain microbleeds, and the like.

Collagen is not an inert member of the ECM landscape, but instead acts as a key component of a variety of a variety of cellular signaling pathways (3). Breakdown of collagen within the ECM may promote unwanted cellular signaling cascades (e.g., proinflammatory or pro-apoptotic) or cell state changes that lead to neurodegeneration. As a hallmark of aging, cellular senescence is a significant contributor to aging and age-related diseases including AD. ECs line the inner wall of blood vessels and are exposed to constant sheer stress from the flow of blood cells (48). EC located at arterial geometries such as curvatures and branches undergo a higher cell turnover, often becoming senescent (49). Atherosclerosis and diabetes are both diseases associated with hypertension, increasing sheer stress and thus cellular senescence of ECs (50, 51). In addition, glycation of collagen type I leads to a premature senescence-like state in endothelial cells (52). Senescent cells adopt an inflammatory senescence-associated secretory phenotype (SASP), releasing excessive pro-inflammatory factors such as interleukin 1β (IL-1β), interleukin-6, and MMPs (49). The accumulation of senescent cells in the brain therefore may result in ECM remodeling and collagen breakdown, triggering inflammatory cell activation and subsequent neurodegeneration (53).

Taken together, the results here provide strong evidence of the therapeutic benefit of CMPs. Thus, CMP treatment of humans and veterinary animals can be expected to result in the treatment, prevention, and perhaps reversal, of a variety of cardiovascular and cerebrovascular pathologies.

### REFERENCES

1 Ritson, M., Wheeler-Jones, C. P. D. & Stolp, H. B. Endothelial dysfunction in neurodegenerative disease: Is endothelial inflammation an overlooked druggable target?Journal of Neuroimmunology 391, 578363 (2024). https://doi.org/https://doi.org/10.1016/j.jneuroim.2024.578363.
2 Ma, Z., Mao, C., Jia, Y., Fu, Y. & Kong, W. Extracellular matrix dynamics in vascular remodeling. American Journal of Physiology-Cell Physiology 319, C481-C499 (2020). https://doi.org/10.1152/ajpcell.00147.2020.
3 Wareham, L. K., Baratta, R. O., Del Buono, B. J., Schlumpf, E. & Calkins, D. J. Collagen in the central nervous system: contributions to neurodegeneration and promise as a therapeutic target. Mol Neurodegener 19, 11 (2024). https://doi.org/10.1186/s13024-024-00704-0.
4 van Varik, B. J. et al. Mechanisms of arterial remodeling: lessons from genetic diseases. Front Genet 3, 290 (2012). https://doi.org/10.3389/fgene.2012.00290
5 Evans, B. R. et al. Inflammatory Mediators in Atherosclerotic Vascular Remodeling. Front Cardiovasc Med 9, 868934 (2022). https://doi.org/10.3389/fcvm.2022.868934.
6 Airhart, N. et al. Smooth muscle cells from abdominal aortic aneurysms are unique and can independently and synergistically degrade insoluble elastin. J Vasc Surg 60, 1033-1041; discussion 1041-1032 (2014). https://doi.org/10.1016/j.jvs.2013.07.097.
7 Grundy, S. M. et al. Prevention Conference VI: Diabetes and Cardiovascular Disease: executive summary: conference proceeding for healthcare professionals from a special writing group of the American Heart Association. Circulation 105, 2231-2239 (2002). https://doi.org/10.1161/01.cir.0000013952.86046.dd
8 Grundy, S. M. et al. Diabetes and cardiovascular disease: a statement for healthcare professionals from the American Heart Association. Circulation 100, 1134-1146 (1999). https://doi.org/10.1161/01.cir.100.10.1134.
9 Wareham, L. K. et al. Collagen mimetic peptide repair of the corneal nerve bed in a mouse model of dry eye disease. Frontiers in Neuroscience 17 (2023). https://doi.org/10.3389/fnins.2023.1148950.
10 Ribeiro, M. et al. Intraocular Delivery of a Collagen Mimetic Peptide Repairs Retinal Ganglion Cell Axons in Chronic and Acute Injury Models. Int J Mol Sci 23 (2022). https://doi.org/10.3390/ijms23062911.
11 Chattopadhyay, S. & Raines, R. T. Collagen-based biomaterials for wound healing. Biopolymers 101, 821-833 (2014). https://doi.org/https://doi.org/10.1002/bip.22486.
12 Chattopadhyay, S. et al. Anchoring a cytoactive factor in a wound bed promotes healing. Journal of Tissue Engineering and Regenerative Medicine 10, 1012-1020 (2016). https://doi.org/https://doi.org/10.1002/term.1886.
13 Chattopadhyay, S., Murphy, C. J., McAnulty, J. F. & Raines, R. T. Peptides that anneal to natural collagen in vitro and ex vivo. Organic &amp; Biomolecular Chemistry 10, 5892 (2012). https://doi.org/10.1039/c2ob25190f.
14 Tucureanu, M. M. et al. Diabetes-induced early molecular and functional changes in aortic heart valves in a murine model of atherosclerosis. Diab Vasc Dis Res 16, 562-576 (2019). https://doi.org/10.1177/1479164119874469.
15 Ganbaatar, B. et al. Empagliflozin ameliorates endothelial dysfunction and suppresses atherogenesis in diabetic apolipoprotein E-deficient mice. Eur J Pharmacol 875, 173040 (2020). https://doi.org/10.1016/j.ejphar.2020.173040.
16 Bink, D. I., Ritz, K., Aronica, E., van der Weerd, L. & Daemen, M. J. Mouse models to study the effect of cardiovascular risk factors on brain structure and cognition. J Cereb Blood Flow Metab 33, 1666-1684 (2013). https://doi.org/10.1038/jcbfm.2013.140.
17 Lo Sasso, G. et al. The Apoe(−/−) mouse model: a suitable model to study cardiovascular and respiratory diseases in the context of cigarette smoke exposure and harm reduction. J Transl Med 14, 146 (2016). https://doi.org/10.1186/s12967-016-0901-1.
18 Ding, H. et al. Endothelial dysfunction in the streptozotocin-induced diabetic apoE-deficient mouse. Br J Pharmacol 146, 1110-1118 (2005). https://doi.org/10.1038/sj.bjp.0706417.
19 Furman, B. L. Streptozotocin-Induced Diabetic Models in Mice and Rats. Curr Protoc 1, e78 (2021). https://doi.org/10.1002/cpzl.78.
20 Bou Ghanem, G. O. et al. Collagen Mimetic Peptides Promote Repair of MMP-1-Damaged Collagen in the Rodent Sclera and Optic Nerve Head. Int J Mol Sci 24 (2023). https://doi.org/10.3390/ijms242317031.
21 Gasecka, A. et al. Early biomarkers of neurodegenerative and neurovascular disorders in diabetes. Journal of Clinical Medicine 9, 2807 (2020).
22 Soni, D., Sagar, P. & Takkar, B. Diabetic retinal neurodegeneration as a form of diabetic retinopathy. International Ophthalmology 41, 3223-3248 (2021).
23 Cicolari, S., Catapano, A. L. & Magni, P. Inflammaging and neurodegenerative diseases: Role of NLRP3 inflammasome activation in brain atherosclerotic vascular disease. Mechanisms of Ageing and Development 195, 111467 (2021).
24 Baradaran, H. et al. The progression of carotid atherosclerosis and imaging markers of dementia. Alzheimer's & Dementia: Translational Research & Clinical Interventions 6, e12015 (2020).
25 Bou Ghanem, G. O., Wareham, L. K. & Calkins, D. J. Addressing neurodegeneration in glaucoma: Mechanisms, challenges, and treatments. Progress in Retinal and Eye Research 100, 101261 (2024). https://doi.org/https://doi.org/10.1016/j.preteyeres.2024.101261.
26 Park, L. et al. Suppression of accelerated diabetic atherosclerosis by the soluble receptor for advanced glycation endproducts. Nat Med 4, 1025-1031 (1998). https://doi.org/10.1038/2012.
27 Ding, H. et al. Endothelial dysfunction in the streptozotocin-induced diabetic apoE-deficient mouse. British journal of pharmacology 146, 1110-1118 (2005).
28 Park, Y.-J., Ko, J. W., Jeon, S. & Kwon, Y. H. Protective effect of genistein against neuronal degeneration in ApoE−/− mice fed a high-fat diet. Nutrients 8, 692 (2016).
29 Bell, R. D. et al. Apolipoprotein E controls cerebrovascular integrity via cyclophilin A. Nature 485, 512-516 (2012).
30 Zhu, Y. et al. Interplay between Extracellular Matrix and Neutrophils in Diseases. Journal of Immunology Research 2021, U.S. Pat. No. 8,243,378 (2021). https://doi.org/https://doi.org/10.1155/2021/8243378.
31 Sasaoka, Y. et al. Study on the mechanism of the blood-glucose-lowering effect of collagen peptides from sturgeon by-products. Marine drugs 19, 584 (2021).
32 Kim, H.-S. et al. Effects of soluble collagen peptides extract derived from Mugil cephalus scale on the blood glucose and lipid metabolism in diabetic rats. Journal of life Science 19, 1794-1801 (2009).
33 Lin, Q. et al. Antidiabetic effect of collagen peptides from harpadon nehereus bones in streptozotocin-induced diabetes mice by regulating oxidative stress and glucose metabolism. Marine Drugs 21, 518 (2023).
34 Wang, J., Zhang, Z., Pei, X., Yang, R. & Li, Y. Effects of marine collagen peptides on blood glucose and lipid metabolism in hyperinsulinemic rats. Wei sheng yan jiu=Journal of hygiene research 39, 143-146 (2010).
35 Zhu, C.-F. et al. Treatment with marine collagen peptides modulates glucose and lipid metabolism in Chinese patients with type 2 diabetes mellitus. Applied Physiology, Nutrition, and Metabolism 35, 797-804 (2010).
36 Devasia, S. et al. Double blind, randomized clinical study to evaluate efficacy of collagen peptide as add on nutritional supplement in Type 2 diabetes. J. Clin. Nutr. Food Sci 1, 006-011 (2018).
37 Kehlet, S. N. et al. Age-related collagen turnover of the interstitial matrix and basement membrane: Implications of age- and sex-dependent remodeling of the extracellular matrix. PLoS One 13, e0194458 (2018). https://doi.org/10.1371/journal.pone.0194458.
38 Matsubayashi, Y. Dynamic movement and turnover of extracellular matrices during tissue development and maintenance. Fly 16, 248-274 (2022). https://doi.org/10.1080/19336934.2022.2076539.
39 Lertkiatmongkol, P., Liao, D., Mei, H., Hu, Y. & Newman, P. J. Endothelial functions of platelet/endothelial cell adhesion molecule-1 (CD31). Curr Opin Hematol 23, 253-259 (2016). https://doi.org/10.1097/MOH.0000000000000239.
40 Nikonenko, A. G., Radenovic, L., Andjus, P. R. & Skibo, G. G. Structural Features of Ischemic Damage in the Hippocampus. The Anatomical Record 292, 1914-1921 (2009). https://doi.org/https://doi.org/10.1002/ar.20969.
41 Kawles, A. et al. Differential vulnerability of the dentate gyrus to tauopathies in dementias. Acta Neuropathologica Communications 11, 1 (2023). https://doi.org/10.1186/s40478-022-01485-7.
42 Zhao, X. & van Praag, H. Steps towards standardized quantification of adult neurogenesis. Nat Commun 11, 4275 (2020). https://doi.org/10.1038/s41467-020-18046-y.
43 Kakoi, C., Udo, H., Matsukawa, T. & Ohnuki, K. Collagen peptides enhance hippocampal neurogenesis and reduce anxietyrelated behavior in mice. Biomedical Research 33, 273-279 (2012).
44 Cheung, K. C. P. et al. Preservation of microvascular barrier function requires CD31 receptor-induced metabolic reprogramming. Nature Communications 11, 3595 (2020). https://doi.org/10.1038/s41467-020-17329-8.
45 Cheung, K. et al. CD31 signals confer immune privilege to the vascular endothelium. Proc Natl Acad Sci USA 112, E5815-5824 (2015). https://doi.org/10.1073/pnas.1509627112.
46 Wareham, L. K. et al. Solving neurodegeneration: common mechanisms and strategies for new treatments. Mol Neurodegener 17, 23 (2022). https://doi.org/10.1186/s13024-022-00524-0.
47 Wareham, L. K. & Calkins, D. J. The Neurovascular Unit in Glaucomatous Neurodegeneration. Front Cell Dev Biol 8, 452 (2020). https://doi.org/10.3389/fcell.2020.00452.
48 Bloom, S. I., Islam, M. T., Lesniewski, L. A. & Donato, A. J. Mechanisms and consequences of endothelial cell senescence. Nature Reviews Cardiology 20, 38-51 (2023). https://doi.org/10.1038/s41569-022-00739-0.
49 Han, Y. & Kim, S. Y. Endothelial senescence in vascular diseases: current understanding and future opportunities in senotherapeutics. Experimental & Molecular Medicine 55, 1-12 (2023). https://doi.org/10.1038/si2276-022-00906-w.
50 Culley, M. K. & Chan, S. Y. Endothelial senescence: a new age in pulmonary hypertension. Circulation research 130, 928-941 (2022).
51 Safaie Qamsari, E. & Stewart, D. J. Cellular senescence in the pathogenesis of pulmonary arterial hypertension: the good, the bad and the uncertain. Frontiers in Immunology 15, 1403669 (2024).
52 Chen, J. et al. Glycated Collagen I Induces Premature Senescence-Like Phenotypic Changes in Endothelial Cells. Circulation Research 90, 1290-1298 (2002). https://doi.org/doi:10.1161/01.RES.0000022161.42655.98.
53 Holloway, K., Neherin, K., Dam, K. U. & Zhang, H. Cellular senescence and neurodegeneration. Human Genetics 142, 1247-1262 (2023). https://doi.org/10.1007/s00439-023-02565-x.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed. For example, the recitation of a range of values (e.g., a range of dosages or dosing concentrations) should be understood to include the values at the beginning and the end of that range, as well as every value in between those beginning and end values. To illustrate this concept, a range of “about 25 ng/ml to about 250 ng/ml” should be interpreted to include a value that is “about 25 ng/ml,” “about 250 ng/ml,” and every individual concentration value between those two values. The term “about” when used in conjunction with a numeric value typically means a value that is the actual value recited ±10% of that value.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Hence, in addition to those specifically described herein, other suitable embodiments of the invention will be readily apparent to one of ordinary skill in the art based upon the foregoing description and examples, and upon knowledge generally available in the relevant arts. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application to which it is relevant.

1-45. (canceled)

46. A composition suitable for use in a medicament for treating or preventing a nervous system disease, disorder or injury in a human or veterinary animal in need of treatment or prevention thereof, said composition comprising (a) at least one collagen mimetic peptide (CMP) and (b) one or more pharmaceutically suitable carriers.

47. The composition of claim 46, wherein said at least one CMP is attached to at least one therapeutic compound (TC) to form a CMP-TC conjugate.

48. The composition of claim 46, wherein said at least one CMP has an amino acid sequence corresponding to any one of SEQ ID NOs:1-388, 397-416, and 418-478.

49. A method of treating or preventing a nervous system disease, disorder or injury in a human or veterinary animal in need of treatment or prevention thereof, comprising administering the composition of claim 46 to said human or veterinary animal in a dosage sufficient to treat or prevent said nervous system disorder or injury, monitoring the status of said disorder or injury in said human or veterinary animal over time, and readministering said composition to the human or veterinary animal until said nervous system disorder or injury is cured, repaired, prevented or ameliorated.

50. The method of claim 49, wherein said nervous system disease, disorder or injury is a central nervous system disease, disorder or injury or a peripheral nervous system disease, disorder or injury.

51. The method of claim 50, wherein said central nervous system disease, disorder or injury is selected from the group consisting of multiple sclerosis, amyotrophic lateral sclerosis, Parkinson's disease, Alzheimer's disease, Huntington's disease, a traumatic encephalopathy, a non-Alzheimer's dementia, encephalitis, chronic traumatic encephalopathy, meningitis, and a neurodegenerative disease or disorder.

52. The method of claim 50, wherein said peripheral nervous system disease, disorder or injury is a peripheral neuropathy or an injury in which one or more peripheral nerves is severed or crushed.

53. The method of claim 49, wherein said composition is administered to the human or veterinary animal parenterally, topically, or in the form of a coating on a solid or semi-solid material that is implanted into one or more tissues or organs of the human or veterinary animal.

54. A medical device suitable for treating or preventing a nervous system disease, disorder or injury in a human or veterinary animal in need of treatment or prevention thereof, wherein said device comprises at least one composition of claim 46.

55. A method of treating, ameliorating or preventing a nervous system disease, disorder or injury in a human or veterinary animal in need of treatment or prevention thereof, said method comprising implanting the medical device of claim 54 into one or more tissues or organs of said human or veterinary animal, and monitoring the medical condition of said human or veterinary animal until said nervous system disease, disorder or injury is cured, repaired, ameliorated or prevented.

56. A composition suitable for use in a medicament for treating or preventing a cardiovascular system disease, disorder or injury in a human or veterinary animal in need of treatment or prevention thereof, said composition comprising (a) at least one collagen mimetic peptide (CMP) and (b) one or more pharmaceutically suitable carriers.

57. The composition of claim 56, wherein said at least one CMP is attached to at least one therapeutic compound (TC) to form a CMP-TC conjugate.

58. The composition of claim 56, wherein said at least one CMP has an amino acid sequence corresponding to any one of SEQ ID NOs:1-388, 397-416, and 418-478.

59. A method of treating or preventing a cardiovascular system disease, disorder or injury in a human or veterinary animal in need of treatment or prevention thereof, comprising administering the composition of claim 56 to said human or veterinary animal in a dosage sufficient to treat or prevent said cardiovascular system disorder or injury, monitoring the status of said disorder or injury in said human or veterinary animal over time, and readministering said composition to the human or veterinary animal until said cardiovascular system disorder or injury is cured, repaired, prevented or ameliorated.

60. The method of claim 59, wherein said cardiovascular system disease, disorder or injury is an atherosclerosis or a disease, disorder or injury of one or more blood vessels of the brain.

61. The method of claim 60, wherein said atherosclerosis is diabetic atherosclerosis.

62. The method of claim 60, wherein said disease, disorder or injury of one or more blood vessels of the brain is selected from the group consisting of stroke, aneurysm, brain hemorrhages, brain microbleeds, and chronic traumatic encephalopathy.

63. The method of claim 59, wherein said composition is administered to the human or veterinary animal parenterally, topically, or in the form of a coating on a solid or semi-solid material that is implanted into one or more tissues or organs of the human or veterinary animal.

64. A medical device suitable for treating or preventing a cardiovascular system disease, disorder or injury in a human or veterinary animal in need of treatment or prevention thereof, wherein said device comprises at least one composition of claim 56.

65. A method of treating, ameliorating or preventing a cardiovascular system disease, disorder or injury in a human or veterinary animal in need of treatment or prevention thereof, said method comprising implanting the medical device of claim 62 into one or more tissues or organs of said human or veterinary animal, and monitoring the medical condition of said human or veterinary animal until said cardiovascular system disease, disorder or injury is cured, repaired, ameliorated or prevented.
